(19) United States
(12) Patent Application Publication
(10) Pub. No.: US 2014/0154459 A1
(43) Pub. Date: Jun. 5, 2014

(54) Fluid-Entangled Laminate Webs Having Hollow Projections and a Process and Apparatus for Making the Same

(71) Applicant: Kimberly-Clark Worldwide, Inc., Neenah, WI (US)

(72) Inventors: Candace Dyan Krautkramer, Neenah, WI (US); Leila Joy Roberson, Kimberly, WI (US); David Glen Biggs, New London, WI (US); Thomas Allan Eby, Greenville, WI (US); Scott S.C. Kirby, Wahroonga (AU); Andy R. Butler, Albert Park (AU); Niall Finn, Lethbridge (AU)

(21) Appl. No.: 13/907,663

(22) Filed: May 31, 2013

Related U.S. Application Data
(63) Continuation-in-part of application No. 13/665,812, filed on Oct. 31, 2012.
Application No. 13/665,812 is a continuation-in-part of application No. 13/664,921, filed on Oct. 31, 2012.

(51) Int. Cl. A61F 13/62; B32B 3/06

(52) U.S. Cl. CPC A61F 13/622; B32B 3/06
USPC 428/99

(57) ABSTRACT

The present invention is directed to a fluid-entangled laminate web and the process and apparatus for its formation as well as end uses for the fluid-entangled laminate web. The laminate web includes a support layer and a nonwoven projection web having a plurality of projections which are preferably hollow. As a result of the fluid-entangling process, entangling fluid is directed through the support layer and into the projection web which is situated on a forming surface. The force of the entangling fluid causes the two layers to be joined to one another and the fluid causes a portion of the fibers in the projection web to be forced into openings present in the forming surface thereby forming the hollow projections. The resultant laminate has a number of uses including, but not limited to, both wet and dry wiping materials, as well as incorporation into various portions of personal care absorbent articles and use in packaging especially food packaging where fluid control is an issue.

### BACKGROUND OF THE INVENTION

Fibrous nonwoven web materials are in wide use in a number of applications, including, but not limited to, absorbent structures and wiping products, many of which are disposable. In particular, such materials are commonly used in personal care absorbent articles such as diapers, diaper pants, training pants, feminine hygiene products, adult incontinence products, bandages, and wiping products such as baby and adult wet wipes. They are also commonly used in cleaning products, such as, wet and dry disposable wipes, which may be treated with cleaning and other compounds which are designed to be used by hand or in conjunction with cleaning devices such as mops. Yet a further application is with beauty aids such as cleansing and make-up removal pads and wipes.

In many of these applications, three-dimensionality and increased surface area are desirable attributes. This is particularly true with body contacting materials for the aforementioned personal care absorbent articles and cleaning products. One of the main functions of personal care absorbent articles is to absorb and retain body exudates such as blood, menses, urine and bowel movements. By providing fibrous nonwovens with hollow projections, several attributes can be achieved at the same time. First, by providing projections, the overall laminate can be made to have a higher degree of thickness while minimizing material used. Increased material thickness serves to enhance the separation of the skin of the user from the absorbent core, hence improving the prospect of drier skin. By providing projections, land areas are created between the projections that can temporarily distance exudates from the high points of the projections while the exudates are being absorbed, thus reducing skin contact and providing better skin benefits. Second, by providing such projections, the spread of exudates in the finished product may be reduced, hence exposing less skin to contamination. Third, by providing projections, the hollows can, themselves, serve as fluid reservoirs to temporarily store body exudates and then later allow the exudates to move vertically into subjacent layers of the overall product. Fourth, by reducing overall skin contact, the fibrous nonwoven laminate with such projections can provide a softer feel to the contacted skin, thereby enhancing the tactile aesthetics of the layer and the overall product. Fifth, when such materials are used as body contacting liner materials for products such as diapers, diaper pants, training pants, adult incontinence products and feminine hygiene products, the liner material also serves the function of acting as a cleaning aid when the product is removed. This is especially the case with menses and lower viscosity bowel movements as are commonly encountered in conjunction with such products. Here again, such materials can provide added benefit from a cleaning and containment perspective.

Fastening systems, such as mechanical fastening systems of the type otherwise referred to as hook and loop fastener systems, have become increasingly widely used in various consumer and industrial applications. A few examples of such applications include disposable personal care absorbent articles, clothing, sporting goods equipment, and a wide variety of other miscellaneous articles. Typically, such hook and loop fastening systems are employed in situations where a refastenable connection between two or more materials or articles is desired. These mechanical fastening systems have in many cases replaced other conventional devices used for making such refastenable connections, such as buttons, buckles, zippers, and the like. Mechanical fastening systems can be advantageously employed in disposable personal care absorbent articles, such as disposable diapers, disposable garments, disposable incontinence products, and the like. Such disposable articles generally are single use items which are discarded after a relatively short period of use—usually a period of hours—and are not intended to be washed and reused.

Mechanical fastening systems typically employ two components—a male (hook) component and a female (loop) component. The hook component usually includes a plurality of semi-rigid, hook-shaped elements anchored or connected to a base material. The loop component generally includes a resilient backing material from which a plurality of upstanding loops project. The hook-shaped elements of the hook component are designed to engage the loops of the loop material, thereby forming mechanical bonds between the hook and loop elements of the two components. These mechanical bonds function to prevent separation of the respective components during normal use. Such mechanical fastening systems are designed to avoid separation of the hook and loop components by application of a shear force or stress, which is applied in a plane parallel to or defined by the connected surfaces of the hook and loop components, as well as certain peel forces or stresses. However, application of a peeling force in a direction generally perpendicular or normal to the place defined by the connected surfaces of the hook and loop components can cause separation of the hook elements from the loop elements, for example, by breaking the loop elements and thereby releasing the engaged hook elements, or by bending the resilient hook elements until the hook elements disengage the loop elements.

With regard to materials which are currently utilized as the female component of a mechanical fastening system, such as, for example, a pattern-unbonded nonwoven web as the “frontal patch” or “landing zone” on the garment facing surface of a personal care absorbent article, such materials are generally stiff and not visually appealing. These materials, such as the pattern-unbonded nonwoven web, are also generally “closed” structures with the fibers generally oriented in the machine direction. Such structures can provide an actual or perceived lack of engagement opportunities for the male component such as a hook fastener. The current female component, such as a pattern-unbonded nonwoven web, also generally has a narrow peel range which is driven by the male component properties.

By providing a fibrous nonwoven with hollow projections to a garment facing surface of a personal care absorbent article as a female component of a mechanical fastening system, several attributes can be achieved at the same time. First, the fibrous nonwoven with such projections can provide a softer feel, thereby enhancing the tactile aesthetics of the female component and of the overall absorbent article. Second, with a fibrous nonwoven with hollow projections as the female component, engagement by a male component can be easier than with current materials. Third, a fibrous nonwoven with hollow projections can provide a more open structure which can provide a higher range of peel strengths. The visual appearance of the hollow projections can also provide the perception of softness and breathability. The fibrous nonwoven with hollow projections can also have greater tensile strength and can therefore provide improved fastening benefits at lower basis weight. The tensile strength of such a fibrous nonwoven can allow for the fibrous nonwoven with hollow projections to undergo various manufacturing and converting processes while still maintaining structure and strength.

In the context of cleaning products, again the projections can provide increased overall surface area for collecting and containing material removed from the surface being cleaned. In addition, cleaning and other compounds may be loaded into the hollow projections to store and then upon use, release these cleaning and other compounds onto the surface being cleaned.

Attempts have been made to provide fibrous nonwoven webs which will provide the above-mentioned attributes and fulfill the above-mentioned tasks. One such approach has been the use of various types of embossing to create three-dimensionality. This works to an extent, however high basis weights are required to create a structure with significant topography. Furthermore, it is inherent in the embossing process that starting thickness is lost due to the fact that embossing is, by its nature, a crushing and bonding process. Furthermore, to “set” the embossments in a nonwoven fabric, the densified sections are typically fused to create weld points that are typically impervious to fluid. Hence a part of the area for fluid to transit through the material is lost. Also, “setting” the fabric can cause the material to stiffen and become harsh to the touch. With regard to engagement of the nonwoven fabric by a hook fastener, creating the weld points diminishes the number of locations in which the hook fasteners can engage the nonwoven fabric. The weld points also convey a perception of a flat and stiff material which can be perceived as less breathable and uncomfortable or potentially irritating due to high stiffness.

Another approach to provide the above-mentioned attributes has been to form fibrous webs on three dimensional forming surfaces. The resulting structures typically have little resilience at low basis weights (assuming soft fibers with desirable aesthetic attributes are used) and the topography is significantly degraded when wound on a roll and put through subsequent converting processes. This is partly addressed in the three-dimensional forming process by allowing the three-dimensional shape to fill with fiber. However, this typically comes at a higher cost due to the usage of more material and at the cost of softness, as well as the fact that the resultant material becomes aesthetically unappealing for certain applications.

Another approach to provide the above-mentioned attributes has been to aperture a fibrous web. Depending on the process, this can generate a flat two-dimensional web or a web with some three-dimensionality where the displaced fiber is pushed out of the plane of the original web. Typically, the extent of the three-dimensionality is limited, and under sufficient load, the displaced fiber may be pushed back toward its original position, resulting in at least partial closure of the aperture. Aperturing processes that attempt to “set” the displaced fiber outside the plane of the original web are also prone to degrading the softness of the starting web. Another problem with apertured materials is that when they are incorporated into end products as this is often done with the use of adhesives, due to their open structure, adhesives will often readily penetrate through the apertures in the nonwoven from its underside to its top, exposed surface, thereby creating unwanted issues such as adhesive build-up in the converting process or creating unintended bonds between layers within the finished product.

As a result, there is still a need for both a material and a process and apparatus which provide three-dimensional characteristics that meet the aforementioned needs. There remains a need for an improved female component for a mechanical fastening system as such are used in personal care absorbent articles. There remains a need for an improved female component to be used as a frontal patch of a mechanical fastening system as such are used in personal care absorbent articles.

### SUMMARY OF THE INVENTION

The present invention is directed to fluid-entangled laminates having a fibrous nonwoven layer with projections which are preferably hollow and which extend from one surface of the laminate as well as the process and apparatus for making such laminates and their incorporation into end products.

The fluid-entangled laminate web according to the present invention, while capable of having other layers incorporated therein, includes a support layer having opposed first and second surfaces and a thickness, and a nonwoven projection web comprising a plurality of fibers and having opposed inner and outer surfaces and a thickness. The second surface of the support layer contacts the inner surface of the projection web and a first plurality of the fibers in the projection web form a plurality of projections which extend outwardly from the outer surface of the projection web. A second plurality of the fibers in the projection web are entangled with the support layer to form the resultant fluid-entangled laminate web.

The projection web portion of the laminate with its projections provides a wide variety of attributes which make it suitable for a number of end uses. In preferred embodiments, all or at least a portion of the projections define hollow interiors.

The support layer can be made from a variety of materials, including a continuous fiber web such as a spunbond material or it can be made from shorter fiber staple fiber webs. The projection web can also be made from both continuous fiber webs and staple fiber webs, though it is desirable for the projection web to have less fiber-to-fiber bonding or fiber entanglement than the support layer to facilitate formation of the projections.

The support layer and the projection web each can be made at a variety of basis weights depending upon the particular end use application. A unique attribute of the laminate, and the process, is the ability to make laminates at what are considered to be low basis weights for applications including, but not limited to, personal care absorbent products and food packaging components. For example, fluid-entangled laminate webs according to the present invention can have overall basis weights between about 25 and about 100 grams per square meter (gsm) and the support layer can have a basis weight of between about 5 and about 40 grams per square meter, while the projection web can have a basis weight of between about 10 and about 60 grams per square meter. Such basis weight ranges are possible due to the manner in which the laminate is formed and the use of two different layers with different functions relative to the formation process. As a result, the laminates are able to be made in commercial settings which heretofore were not considered possible due to the inability to process the individual webs and form the desired projections.

The laminate web according to the present invention can be incorporated into absorbent articles for a wide variety of uses including, but not limited to, diapers, diaper pants, training pants, incontinence devices, feminine hygiene products, bandages and wipes. Typically, such products will include a body side liner or skin-contacting material, a garment-facing material also referred to as a backsheet and an absorbent core disposed between the body side liner and the backsheet. In this regard, such absorbent articles can have at least one layer which is made, at least in part, of the fluid-entangled laminate web of the present invention, including, but not limited to, one of the external surfaces of the absorbent article. If the external surface is the body contacting surface, the fluid entangled laminate web can be used alone or in combination with other layers of absorbent material. In addition, the fluid-entangled laminate web may include hydrogel, also known as superabsorbent material, preferably in the support layer portion of the laminate. If the laminate web is to be used as an external surface on the garment side of the absorbent article, it may be desirable to attach a liquid impermeable layer such as a layer of film to the first or exterior surface of the support layer and position this liquid impermeable layer to the inward side of the absorbent article so the projections of the projection web are on the external side of the absorbent article. This same type of configuration can also be used in food packaging to absorb fluids from the contents of the package.

It is also very common for such absorbent articles to have an optional layer which is commonly referred to as a “surge” or “transfer” layer disposed between the body side liner and the absorbent core. When such products are in the form of, for example, diapers and adult incontinence devices, they can also include what are termed “ears” located in the front and/or back waist regions at the lateral sides of the products. These ears are used to secure the product about the torso of the wearer, typically in conjunction with adhesive and/or mechanical fastening systems having male and female components such as hook and loop fastening systems. In certain applications, the male component of the fastening systems are connected to the distal ends of the ears and are attached to a female component, such as what is referred to as a “frontal patch” or “tape landing zone” located on the front waist portion of the product. The fluid-entangled laminate web according to the present invention may be used for all or a portion of any one or more of these components and products.

When such absorbent articles are in the form of, for example, a training pant, diaper pant or other product which is designed to be pulled on and worn like underwear, such products will typically include what are termed “side panels” joining the front and back waist regions of the product. Such side panels can include both elastic and non-elastic portions and the fluid-entangled laminate webs of the present invention can be used as all or a portion of these side panels as well.

Consequently, such absorbent articles can have at least one layer, all or a portion of which, comprises the fluid entangled laminate web of the present invention.

Also disclosed herein are a number of equipment configurations and processes for forming fluid-entangled laminate webs according to the present invention. One such process includes the process steps of providing a projection forming surface defining a plurality of forming holes therein with the forming holes being spaced apart from one another and having land areas therebetween. The projection forming surface is capable of movement in a machine direction at a projection forming surface speed. A projection fluid entangling device is also provided which has a plurality of projection fluid jets capable of emitting a plurality of pressurized projection fluid streams from the projection fluid jets in a direction towards the projection forming surface.

A support layer having opposed first and second surfaces and a nonwoven projection web having a plurality of fibers and opposed inner and outer surfaces are next provided. The projection web is fed onto the projection forming surface with the outer surface of the projection web positioned adjacent to the projection forming surface. The second surface of the support layer is fed onto the inner surface of the projection web. A plurality of pressurized projection fluid streams of the entangling fluid from the plurality of projection fluid jets are directed in a direction from the first surface of the support layer towards the projection forming surface to cause a) a first plurality of the fibers in the projection web in a vicinity of the forming holes in the projection forming surface to be directed into the forming holes to form a plurality of projections extending outwardly from the outer surface of the projection web, and b) a second plurality of the fibers in the projection web to become entangled with the support layer to form a laminate web. This entanglement may be the result of the fibers of the projection web entangling with the support layer, or, when the support layer is a fibrous structure too, fibers of the support layer entangling with the fibers of the projection web, or a combination of the two described entanglement processes. In addition, the first and second plurality of fibers in the projection web may be the same plurality of fibers, especially when the projections are closely spaced as the same fibers, if of sufficient length, can both form the projections and entangle with the support layer.

Following the formation of the projections in the projection web and the attachment of the projection web with the support layer to form the laminate web, the laminate web is removed from the projection forming surface. In certain executions of the process and apparatus it is desirable that the direction of the plurality of fluid streams causes the formation of projections which are hollow.

In a preferred design, the projection forming surface comprises a texturizing drum though it is also possible to form the forming surface from a belt system or belt and wire system. In certain executions, it is desirable that the land areas of the projection forming surface not be fluid permeable, in other situations they can be permeable, especially when the forming surface is a porous forming wire. If desired, the forming surface can be formed with raised areas in addition to the holes so as to form depressions and/or apertures in the land areas of the fluid-entangled laminate web according to the present invention.

In alternate executions of the equipment, the projection web and/or the support layer can be fed into the projection forming process at the same speed as the projection forming surface is moving or at a faster or slower rate. In certain executions of the process, it is desirable that the projection web be fed onto the projection forming surface at a speed which is greater than a speed the support layer is fed onto the projection web. In other situations, it may be desirable to feed both the projection web and the support layer onto the projection forming surface at a speed which is greater than the speed of the projection forming surface. It has been found that overfeeding material into the process provides additional fibrous structure within the projection web for formation of the projections. The rate at which the material is fed into the process is called the overfeed ratio. It has been found that particularly well-formed projections can be made when the overfeed ratio is between about 10 and about 50 percent, meaning that the speed at which the material is fed into the process and apparatus is between about 10 percent and about 50 percent faster than the speed of the projection forming surface. This is particularly advantageous with respect to the overfeeding of the projection web into the process and apparatus.

In an alternate form of the process and equipment, a pre-lamination step is provided in advance of the projection forming step. In this embodiment, the equipment and process are provided with a lamination forming surface which is permeable to fluids. The lamination forming surface is capable of movement in a machine direction at a lamination forming speed. As with the other embodiment of the process and equipment, a projection forming surface is provided which defines a plurality of forming holes therein with the forming holes being spaced apart from one another and having land areas therebetween. The projection forming surface is also capable of movement in the machine direction at a projection forming surface speed. The equipment and process also include a lamination fluid entangling device having a plurality of lamination fluid jets capable of emitting a plurality of pressurized lamination fluid streams of entangling fluid from the lamination fluid jets in a direction toward the lamination forming surface and a projection fluid entangling device having a plurality of projection fluid jets capable of emitting a plurality of pressurized projection fluid streams of an entangling fluid from the projection fluid jets in a direction towards the projection forming surface.

As with the other process and equipment, a support layer having opposed first and second surfaces and a projection web having a plurality of fibers and opposed inner and outer surfaces are next provided. The support layer and the projection web are fed onto the lamination forming surface at which point a plurality of pressurized lamination fluid streams of entangling fluid are directed from the plurality of lamination fluid jets into the support layer and the projection web to cause at least a portion of the fibers from the projection web to become entangled with the support layer to form a laminate web.

After the laminate web is formed, it is fed onto the projection forming surface with the outer surface of the projection web being adjacent the projection forming surface. Next, a plurality of pressurized projection fluid streams of the entangling fluid from the plurality of projection fluid jets are directed into the laminate web in a direction from the first surface of the support layer towards the projection forming surface to cause a first plurality of the fibers in the projection web in a vicinity of the forming holes in the projection forming surface to be directed into the forming holes to form a plurality of projections extending outwardly from the outer surface of the projection web. The thus formed fluid-entangled laminate web is then removed from the projection forming surface.

In the process which employs a lamination step prior to the projection forming step, the lamination may take place with either the support layer being the layer which is in direct contact with the lamination forming surface or with the projection web being in direct contact with the lamination forming surface. When the support layer is fed onto the lamination forming surface, its first surface will be adjacent the lamination forming surface and so the inner surface of the projection web is thus fed onto the second surface of the support layer. As a result, the plurality of pressurized lamination fluid streams of entangling fluid emanating from the pressurized lamination fluid jets are directed from the outer surface of the projection web towards the lamination forming surface to cause at least a portion of the fibers from the projection web to become entangled with the support layer to form the laminate web.

As with the first process, the projection forming surface may comprise a texturizing drum and in certain applications it is desirable that the land areas of the projection forming surface not be fluid permeable relative to the entangling fluid being used. It is also desirable that the plurality of pressurized projection fluid streams cause the formation of projections which are hollow. In addition, the projection web can be fed onto the support layer at a speed that is greater than the speed the support layer is fed onto the lamination forming surface. Alternatively, both the projection web and the support layer can be fed onto the lamination forming surface at a speed that is greater than the lamination forming surface speed. The overfeed ratio for the material being fed into the lamination forming portion of the process can be between about 10 and about 50 percent. Once the laminate web has been formed, it can be fed onto the projection forming surface at a speed that is greater than the projection forming surface speed.

In some applications, it may be desirable that the projections have additional rigidity and abrasion resistance such as when the laminate web is used as a cleansing pad or where the projections and the overall laminate will see more vertical compressive forces. In such situations, it may be desirable to form the projection web with fibers which are able to bond or be bonded to one another such as by the use, for example, of bicomponent fibers. Alternatively or in addition thereto, chemical bonding, such as through the use of acrylic resins, can be used to bond the fibers together. In such situations, the laminate web may be subjected to further processing such as a bonding step wherein the newly formed laminate is subjected to a heating or other non-compressive bonding process which fuses all or a portion of the fibers in the projections and, if desired, in the surrounding areas together to give the laminate more structural rigidity.

These and other embodiments of the present invention are set forth in further detail below.

### BRIEF DESCRIPTION OF THE DRAWINGS

A full and enabling disclosure of the present invention, including the best mode thereof, is set forth more particularly in the remainder of the specification, which includes reference to the accompanying figures, in which:

FIG. 1 is a perspective view of one embodiment of a fluid entangled laminate web according to the present invention.

FIG. 2 is a cross-section of the material shown in FIG. 1 taken along line 2-2 of FIG. 1.

FIG. 2A is a cross-sectional view of the material according to the present invention taken along line 2-2 of FIG. 1 showing possible directions of fiber movements within the laminate due to the fluid-entanglement process according to the present invention.

FIG. 3 is a schematic side view of an apparatus and process according to the present invention for forming a fluid-entangled laminate web according to the present invention.

FIG. 3A is an exploded view of a representative portion of a projection forming surface according to the present invention.

FIG. 4 is a schematic side view of an alternate apparatus and process according to the present invention for forming a fluid-entangled laminate web according to the present invention.

FIG. 4A is a schematic side view of an alternate apparatus and process according to the present invention for forming a fluid-entangled laminate web according to the present invention which is an adaptation of the apparatus and process shown in FIG. 4 as well as subsequent FIGS. 5 and 7.

FIG. 5 is a schematic side view of an alternate apparatus and process according to the present invention for forming a fluid-entangled laminate web according to the present invention.

FIG. 6 is a schematic side view of an alternate apparatus and process according to the present invention for forming a fluid-entangled laminate web according to the present invention.

FIG. 7 is a schematic side view of an alternate apparatus and process according to the present invention for forming a fluid-entangled laminate web according to the present invention.

FIG. 8 is a photomicrograph at a 45 degree angle showing a fluid-entangled laminate web according to the present invention.

FIGS. 9 and 9A are photomicrographs showing in cross-section a fluid-entangled laminate web according to the present invention.

FIG. 10 is a perspective cutaway view of an absorbent article in an unfastened, stretched and laid-flat condition in which a fluid-entangled laminate web according to the present invention can be used.

FIG. 11 is a side view illustration of an embodiment of an absorbent article.

FIG. 12 is a plan view of a non-limiting illustration of an absorbent article, such as, for example, a diaper, in an unfastened, stretched and laid-flat configuration with the surface of the absorbent article which contacts the wearer facing the viewer and with portions cut away for clarity of illustration.

FIG. 13 is an optical photo in top view of a pattern-unbonded nonwoven material with a horizontal field width of 14.0 mm.

FIG. 14 is an optical photo in top view of a fluid-entangled laminate web according to the present invention with a horizontal field width of 14.0 mm.

FIG. 15 is a SEM image of the top view of a dome of a pattern-unbonded nonwoven web.

FIG. 16 is a SEM image of the top view of a fluid-entangled laminate web according to the present invention.

FIG. 17 is a perspective view illustration of an embodiment of an absorbent article.

FIG. 18 is a perspective view of an exemplary illustration of a set-up of an imaging system used for determining the percent open area.

FIG. 19 is a perspective view of an exemplary illustration of a set-up of an imaging system used for determining projection height.

FIG. 20 is an illustration of the approximate sampling position required during imaging analysis of fiber orientation according to the Method to Determine Orientation described herein.

FIG. 21 is an illustration of the approximate sampling position and the image that results when analyzing the percentage of void space according to the Method to Determine Percent Void Space described herein.

FIG. 22 is a graph depicting fabric thickness as a function of the overfeed ratio of the projection web into the forming process.

FIG. 23 is a graph depicting fabric extension at a 10N load as a function of the overfeed ratio of the projection web into the forming process for both laminates according to the present invention and unsupported projection webs.

FIG. 24 is a graph depicting the load in Newtons per 50 millimeters width as a function of the percent extension comparing both a laminate according to the present invention and unsupported projection web.

FIG. 25 is a graph depicting the load in Newtons per 50 mm width as a function of the percent strain for a series of laminates according to the present invention while varying the overfeed ratio.

FIG. 26 is a graph depicting the load in Newtons per 50 mm width as a function of the percent extension for a series of 45 gsm projection webs while varying the overfeed ratio.

FIG. 27 is a photo in top view of a sample designated as code 3-6 in Table 2 of the specification.

FIG. 27A is a photo of a sample designated as code 3-6 in Table 2 of the specification taken at a 45 degree angle.

FIG. 28 is a photo in top view of a sample designated as code 5-3 in Table 2 of the specification.

FIG. 28A is a photo of a sample designated as code 5-3 in Table 2 of the specification taken at a 45 degree angle.

FIG. 29 is a photo showing the juxtaposition of a portion of a fabric with and without a support layer backing the projection web having been processed simultaneously on the same machine.

FIG. 30 is a graph depicting the peel strength for a series of laminates.

FIG. 31 is a graph depicting the shear strength for a series of laminates.

FIG. 32 is a graph depicting the student's T confidence limit of the ranges of percent void space in the projections of a series of laminates at the 90% confidence level.

FIG. 33 is a graph depicting the student's T confidence limit of the ranges of field orientation of a series of laminates at the 90% confidence level.

FIG. 34 is a graph depicting the student's T confidence limit of the ranges of field orientation rotational percent relative standard deviation of a series of laminates at the 90% confidence level.

FIG. 35 is a graph depicting the student's T confidence limit of the ranges of fiber segment orientation of a series of laminates at the 90% confidence level.

FIG. 36 is a graph depicting the student's T confidence limit of the ranges of fiber segment orientation rotational percent relative standard deviation of a series of laminates at the 90% confidence level.

FIG. 37 is a graph depicting the shear strength versus the tensile load in the machine direction for a series of laminates.

Repeat use of reference characters in the present specification and drawings is intended to represent the same or analogous features or elements of the present invention.

### DETAILED DESCRIPTION OF REPRESENTATIVE EMBODIMENTS

### Definitions

As used herein, the term “absorbent article” generally refers to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads, incontinence products, medical garments, surgical pads and bandages, other personal care or health care garments, and the like without departing from the scope of the present disclosure.

As used herein, the term “bonded” generally refers to the joining, adhering, connecting, attaching, or the like, of two elements. Two elements will be considered bonded together when they are joined, adhered, connected, attached, or the like, directly to one another or indirectly to one another, such as when each is directly bonded to intermediate elements. The bonding can occur via continuous or intermittent bonds.

As used herein, the term “carded web” generally refers to a web containing natural or synthetic staple length fibers typically having fiber lengths less than 100 millimeters. Bales of staple fibers undergo an opening process to separate the fibers which are then sent to a carding process which separates and combs the fibers to align them in the machine direction after which the fibers are deposited onto a moving wire for further processing. Such webs usually are subjected to some type of bonding process such as thermal bonding using heat and/or pressure. In addition or in lieu thereof, the fibers may be subject to adhesive processes to bind the fibers together such as by the use of powder adhesives. Still further, the carded web may be subjected to fluid entangling such as hydroentangling to further intertwine the fibers and thereby improve the integrity of the carded web. Carded webs due to the fiber alignment in the machine direction, once bonded, will typically have more machine direction strength than cross machine direction strength.

As used herein, the term “film” generally refers to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

As used herein, the term “fluid entangling” and “fluid-entangled” generally refers to a formation process for further increasing the degree of fiber entanglement within a given fibrous nonwoven web or between fibrous nonwoven webs and other materials so as to make the separation of the individual fibers and/or the layers more difficult as a result of the entanglement. Generally, this is accomplished by supporting the fibrous nonwoven web on some type of forming or carrier surface which has at least some degree of permeability to the impinging pressurized fluid. A pressurized fluid stream (usually multiple streams) is then directed against the surface of the nonwoven web which is opposite the supported surface of the web. The pressurized fluid contacts the fibers and forces portions of the fibers in the direction of the fluid flow, thus displacing all or a portion of a plurality of the fibers towards the supported surface of the web. The result is a further entanglement of the fibers in what can be termed the Z-direction of the web (its thickness) relative to its more planar dimension, its X-Y plane. When two or more separate webs or other layers are placed adjacent one another on the forming/carrier surface and subjected to the pressurized fluid, the generally desired result is that some of the fibers of at least one of the webs are forced into the adjacent web or layer, thereby causing fiber entanglement between the interfaces of the two surfaces so as to result in the bonding or joining of the webs/layers together due to the increased entanglement of the fibers. The degree of bonding or entanglement will depend on a number of factors including, but not limited to, the types of fibers being used, their fiber lengths, the degree of pre-bonding or entanglement of the web or webs prior to subjection to the fluid entangling process, the type of fluid being used (liquids, such as water, steam or gases, such as air), the pressure of the fluid, the number of fluid streams, the speed of the process, the dwell time of the fluid and the porosity of the web or webs/other layers and the forming/carrier surface. One of the most common fluid entangling processes is referred to as hydroentangling, which is a well-known process to those of ordinary skill in the art of nonwoven webs. Examples of fluid entangling processes can be found in U.S. Pat. No. 4,939,016 to Radwanski et al., U.S. Pat. No. 3,485,706 to Evans, and U.S. Pat. Nos. 4,970,104 and 4,959,531 to Radwanski, each of which is incorporated herein in its entirety by reference thereto for all purposes.

As used herein, the term “g/cc” generally refers to grams per cubic centimeter.

As used herein, the term “gsm” generally refers to grams per square meter.

As used herein, the term “hydrophilic” generally refers to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by the Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

As used herein, the term “liquid impermeable” generally refers to a layer or multi-layer laminate in which liquid body exudates, such as urine, will not pass through the layer or laminate, under ordinary use conditions, in a direction generally perpendicular to the plane of the layer or laminate at the point of liquid contact.

As used herein, the term “liquid permeable” generally refers to any material that is not liquid impermeable.

As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein the term “nonwoven fabric or web” refers to a web having a structure of individual fibers, filaments or threads (collectively referred to as “fibers” for sake of simplicity) which are interlaid, but not in an identifiable manner as in a knitted fabric. Nonwoven fabrics or webs have been formed from many processes, such as, for example, meltblowing processes, spunbonding processes, carded web processes, etc.

As used herein, the term “pliable” generally refers to materials which are compliant and which will readily conform to the general shape and contours of the wearer's body.

As used herein, the term “spunbond web” generally refers to a web containing small diameter, substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 microns, and are often between about 5 to about 20 microns. To provide additional web integrity, the webs so formed can be subjected to additional fiber bonding techniques if so desired. See for example, U.S. Pat. No. 3,855,046 to Hansen et al., which is incorporated herein in its entirety by reference thereto for all purposes.

As used herein, the term “superabsorbent” generally refers to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

As used herein, the term “surge layer” generally refers to a layer capable of accepting and temporarily holding liquid body exudates to decelerate and diffuse a surge or gush of the liquid body exudates and to subsequently release the liquid body exudates therefrom into another layer or layers of the absorbent article.

As used herein, the term “thermoplastic” generally refers to a material which softens and which can be shaped when exposed to heat and which substantially returns to a non-softened condition when cooled.

The term “user” refers herein to one who fits an absorbent article, such as, but not limited to, a diaper, diaper pants, training pant, youth pant, incontinent product, or other absorbent article about the wearer of one of these absorbent articles. A user and a wearer can be one and the same person.

### DETAILED DESCRIPTION OF THE INVENTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. When ranges for parameters are given, it is intended that each of the endpoints of the range are also included within the given range. It is to be understood by one of ordinary skill in the art that the present discussion is a description of exemplary embodiments only, and is not intended as limiting the broader aspects of the present invention, which broader aspects are embodied exemplary constructions.

Fluid-Entangled Laminate Web with Projections

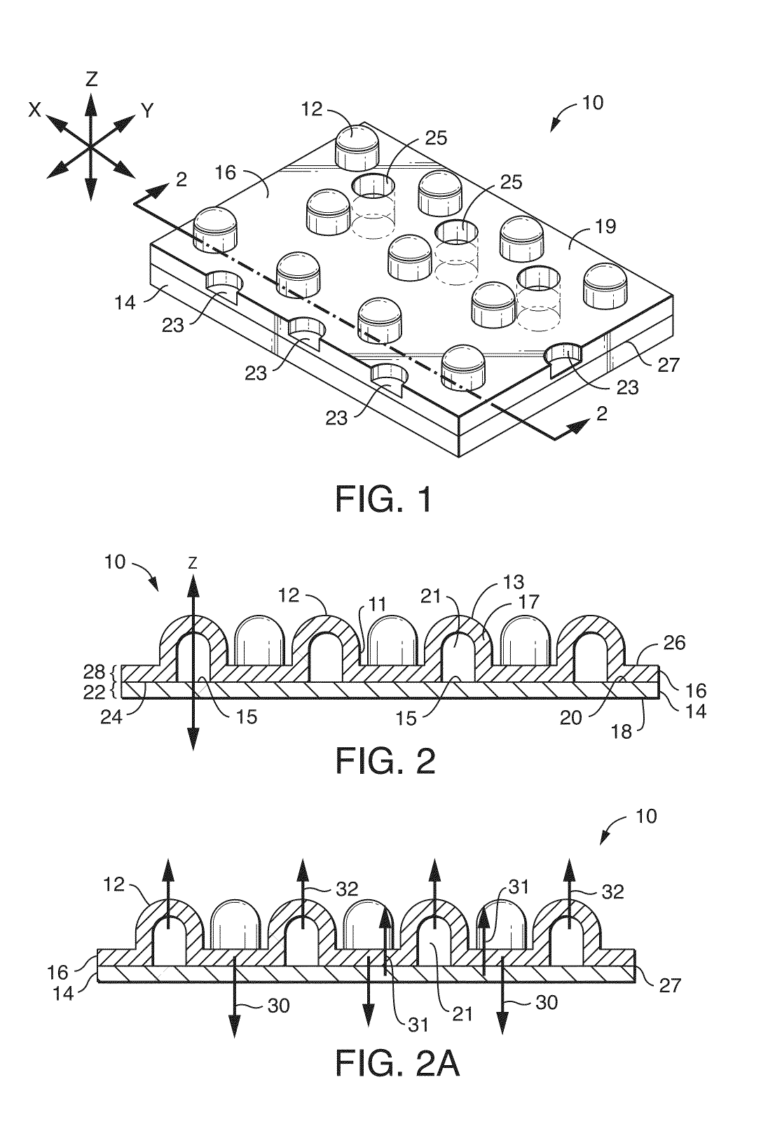

The result of the processes and apparatus described herein is the generation of a fluid-entangled laminate web with projections extending outwardly and away from at least one intended external surface of the laminate. In preferred embodiments the projections are hollow. An embodiment of the present invention is shown in FIGS. 1, 2, 2A, 8, 9 and 9A of the drawings. A fluid-entangled laminate web 10 is shown with projections 12 which for many applications are desirably hollow. The web 10 includes a support layer 14 (which in FIGS. 1, 2 and 2A is shown as a fibrous nonwoven support layer 14) and a fibrous nonwoven projection web 16. The support layer 14 has a first surface 18 and an opposed second surface 20, as well as a thickness 22. The projection web 16 has an inner surface 24 and an opposed outer surface 26, as well as a thickness 28. The interface between the support layer 14 and the projection web 16 is shown by reference number 27 and it is desirable that the fibers of the projection web 16 cross the interface 27 and be entangled with and engage the support layer 14 so as to form the laminate 10. When the support layer or web 14 is also a fibrous nonwoven, the fibers of this layer may cross the interface 27 and be entangled with the fibers in the projection web 16. The overall laminate 10 is referred to as a fluid-entangled laminate web due to the fibrous nature of the projection web 16 portion of the laminate 10 while it is understood that the support layer 14 is referred to as a layer as it may comprise fibrous web material such as nonwoven material but it also may comprise or include other materials such as, for example, films, scrims and foams. Generally, for the end-use applications outlined herein, basis weights for the fluid-entangled laminate web 10 will range between about 25 and about 100 gsm, though basis weights outside this range may be used depending upon the particular end-use application.

### Hollow Projections

While the projections 12 can be filled with fibers from the projection web 16 and/or the support layer 14, it is generally desirable for the projections 12 to be generally hollow, especially when such laminates 10 are being used in connection with absorbent structures. The hollow projections 12 desirably have closed ends 13 which are devoid of holes or apertures. Such holes or apertures are to be distinguished from the normal interstitial fiber-to-fiber spacing commonly found in fibrous nonwoven webs. In some applications, however, it may be desirable to increase the pressure and/or dwell time of the impinging fluid jets in the entangling process as described below to create one or more holes or apertures (not shown) in one or more of the hollow projections 12. Such apertures may be formed in the ends 13 or side walls 11 of the projections 12 as well as in both the ends 13 and side walls 11 of the projections 12.

In various embodiments, the projections 12 can have a percentage of open area in which light can pass through the projections 12 unhindered by the material forming the projections 12, such as, for example, fibrous material. The percentage of open area present in the projections 12 encompasses all area of the projection 12 where light can pass through the projection 12 unhindered. Thus, for example, the percentage of open area of a projection 12 can encompass all open area of the projection 12 via apertures, interstitial fiber-to-fiber spacing, and any other spacing within the projection 12 where light can pass through unhindered. In an embodiment, the projections 12 can be formed without apertures and the open area can be due to the interstitial fiber-to-fiber spacing. In various embodiments, the projections 12 can have less than about 1, 0.9, 0.8, 0.7, 0.6, 0.5, 0.4, 0.3, 0.2, or 0.1% open area in a chosen area of the laminate web 10 as measured according to the Method to Determine Percent Open Area test method described herein.

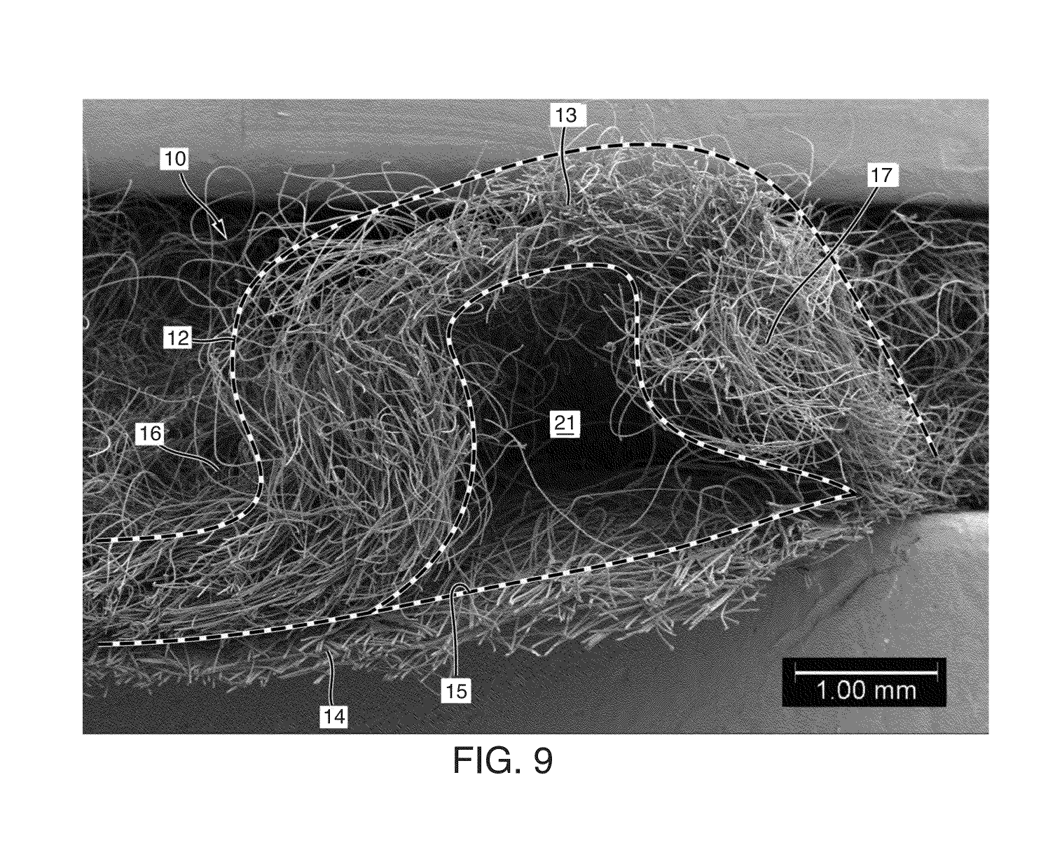

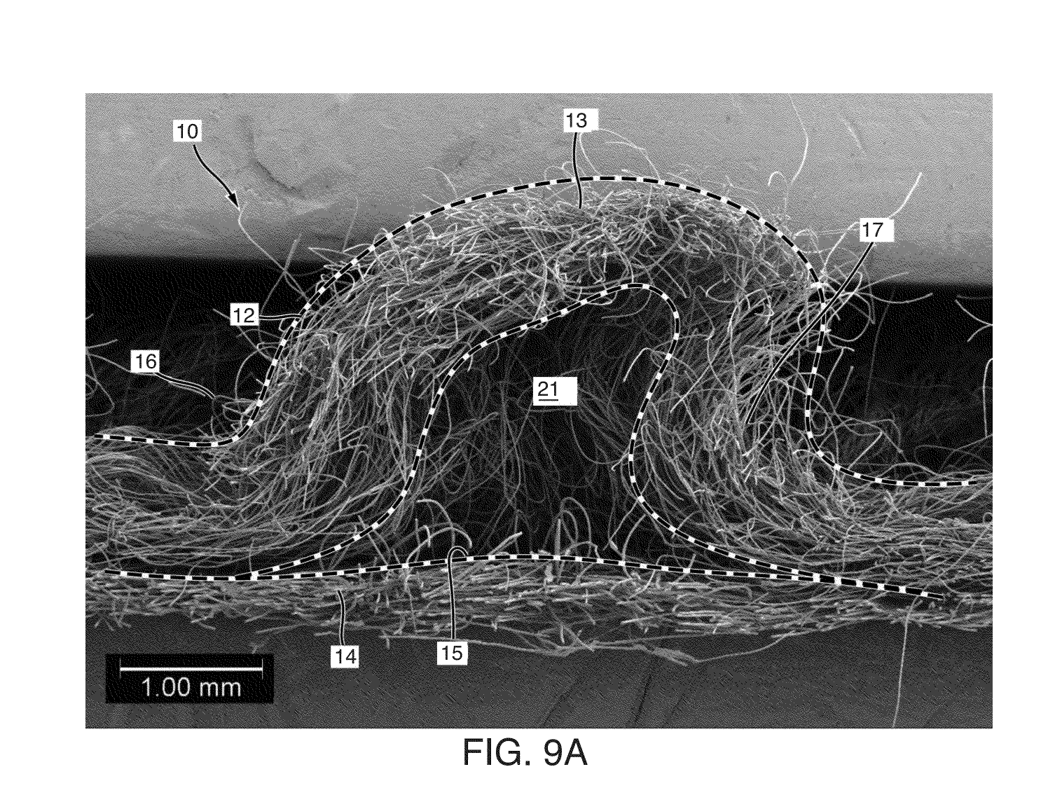

The hollow projections 12, shown in a non-limiting embodiment in FIG. 8, are round when viewed from above with somewhat domed or curved tops or ends 13, such as seen when viewed in the cross-section, such as shown in FIGS. 9 and 9A. The actual shape of the projections 12 can be varied depending on the shape of the forming surface into which the fibers from the projection web 16 are forced. Thus, while not limiting the variations, the shapes of the projections 12 may be, for example, round, oval, square, rectangular, triangular, diamond-shaped, etc. Both the width and depth of the hollow projections 12 can be varied as can be the spacing and pattern of the projections 12. Further, various shapes, sizes and spacing of the projections 12 can be utilized in the same projection web 16. In an embodiment, the projections 12 can have a height, measured according to the Method to Determine Height of Projections test method described herein, of greater than about 1 mm. In an embodiment, the projections 12 can have a height greater than about 1, 2, 3, 4, 5, 6, 7, 8, 9, or 10 mm. In an embodiment, the projections 12 can have a height from about 1, 2, 3, 4, or 5 mm to about 6, 7, 8, 9 or 10 mm.

The projections 12 in the laminate web 10 are located on and emanate from the outer surface 26 of the projection web 16. When the projections 12 are hollow, they will have open ends 15, which are located towards the inner surface 24 of the projection web 16 and are covered by the second surface 20 of the support layer or web 14 or the inner surface 24 of the projection web 16, depending upon the amount of fiber that has been used from the projection web 16 to form the projections 12. The projections 12 are surrounded by land areas 19, which are also formed from the outer surface 26 of the projection web 16, though the thickness of the land areas 19 is comprised of both the projection web 16 and the support layer 14. This land area 19 may be relatively flat and planar, as shown in FIGS. 1 and 2, or it may have topographical variability built into it. For example, the land area 19 may have a plurality of three-dimensional shapes formed into it by forming the projection web 16 on a three-dimensionally-shaped forming surface such as is disclosed in U.S. Pat. No. 4,741,941 to Englebert et al., assigned to Kimberly-Clark Worldwide and incorporated herein by reference in its entirety for all purposes. For example, the land areas 19 may be provided with depressions 23 which extend all or part way into the projection web 16 and/or the support layer 14. In addition, the land areas 19 may be subjected to embossing which can impart surface texture and other functional attributes to the land area 19. Still further, the land areas 19 and the laminate 10 as a whole may be provided with apertures 25 which extend through the laminate 10 so as to further facilitate the movement of fluids (such as the liquids and solids that make up body exudates) into and through the laminate 10. As a result of the fluid entanglement processes described herein, it is generally not desirable that the fluid pressure used to form the projections 12 be of sufficient force so as to force fibers from the support layer 14 to be exposed on the outer surface 26 of the projection web 16.

In various embodiments, the land areas 19 can have a percentage of open area in which light can pass through the land areas 19 unhindered by the material forming the land areas 19, such as, for example, fibrous material. The percentage of open area present in the land areas 19 encompasses all area of the land areas 19 where light can pass through the land areas 19 unhindered. Thus, for example, the percentage of open area of a land area 19 can encompass all open area of the land areas 19 via apertures, interstitial fiber-to-fiber spacing, and any other spacing within the land areas 19 where light can pass through unhindered. In an embodiment, the land areas 19 can be formed without apertures and the open area can be due to the interstitial fiber-to-fiber spacing. In various embodiments, the land areas 19 can have greater than about 1% open area in a chosen area of the laminate web 10, as measured according to the Method to Determine Percent Open Area test method described herein. In various embodiments, the land areas 19 can have greater than about 1, 2, 3, 4, 5, 6, 7, 8, 9, 10, 11, 12, 13, 14, 15, 16, 17, 18, 19 or 20% open area in a chosen area of the laminate web 10. In various embodiments, the land areas 19 can have about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20% open area in a chosen area of the laminate web 10. In various embodiments, the land areas 19 can have from about 1, 2 or 3% to about 4 or 5% open area in a chosen area of the laminate web 10. In various embodiments, the land areas 19 can have from about 5, 6 or 7% to about 8, 9 or 10% open area in a chosen area of the laminate web 10. In various embodiments, the land areas 19 can have from about 10, 11, 12, 13, 14 or 15% to about 16, 17, 18, 19 or 20% open area in a chosen area of the laminate web 10. In various embodiments, the land areas 19 can have greater than about 20% open area in a chosen area of the laminate web 10.

While it is possible to vary the density and fiber content of the projections 12, it is generally desirable that the projections 12 be “hollow”. Referring to FIGS. 9 and 9A, it can be seen that when the projections 12 are hollow, they tend to form a shell 17 from the fibers of the projection web 16. The shell 17 defines an interior hollow space 21 which has a lower density of fibers as compared to the density of the shell 17 of the projections 12. By “density” it is meant the fiber count or content per chosen unit of volume within a portion of the interior hollow space 21 or the shell 17 of the projections 12. The thickness of the shell 17, as well as its density, may vary within a particular or individual projection 12 and it also may vary as between different projections 12. In addition, the size of the hollow interior space 21, as well as its density, may vary within a particular or individual projection 12 and it also may vary as between different projections 12. The photomicrographs of FIGS. 9 and 9A reveal a lower density or count of fibers in the interior hollow space 21 as compared to the shell portion 17 of the illustrated projection 12. As a result, if there is at least some portion of an interior hollow space 21 of a projection 12 that has a lower fiber density than at least some portion of the shell 17 of the same projection 12, then the projection is regarded as being “hollow”. In this regard, in some situations, there may not be a well-defined demarcation between the shell 17 and the interior hollow space 21 but, if with sufficient magnification of a cross-section of one of the projections, it can be seen that at least some portion of the interior hollow space 21 of the projection 12 has a lower density than some portion of the shell 17 of the same projection 12, then the projection 12 is regarded as being “hollow”. Further if at least a portion of the projections 12 of a fluid-entangled laminate web 10 are hollow, the projection web 16 and the laminate 10 are regarded as being “hollow” or as having “hollow projections”. Typically the portion of the projections 12 which are hollow will be greater than or equal to 50 percent of the projections 12 in a chosen area of the fluid-entangled laminate web 10, alternatively, greater than or equal to 70 percent of the projections in a chosen area of the fluid-entangled laminate web 10 and, alternatively, greater than or equal to 90 percent of the projections 10 in a chosen area of the fluid-entangled laminate web 10.

As will become more apparent in connection with the description of the processes set forth below, the fluid-entangled laminate web 10 is the result of the movement of the fibers in the projection web 16 in one and sometimes two or more directions. Referring to FIGS. 1, 2, 2A and 3A, if the projection forming surface 130 upon which the projection web 16 is placed is solid, except for the forming holes or apertures 134 used to form the hollow projections 12, then the force of the fluid entangling streams hitting and rebounding off the solid surface area 136 of the projection forming surface 130 corresponding to the land areas 19 of the projection web 16 can cause a migration of fibers adjacent the inner surface 24 of the projection web 16 into the support layer 14 adjacent its second surface 20. This migration of fibers in the first direction is represented by the arrows 30 shown in FIG. 2A. In order to form the hollow projections 12 extending outwardly from the outer surface 26 of the projection web 16, there must be a migration of fibers in a second direction as shown by the arrows 32. It is this migration in the second direction which causes fibers from the projection web 16 to move out and away from the outer surface 26 to form the hollow projections 12.

When the support layer 14 is a fibrous nonwoven web, depending on the degree of web integrity and the strength and dwell time of the entangling fluid from the pressurized fluid jets, there also may be a movement of support web fibers into the projection web 16 as shown by arrows 31 in FIG. 2A. The net result of these fiber movements is the creation of a laminate 10 with good overall integrity and lamination of the layer and web (14 and 16) at their interface 27, thereby permitting further processing and handling of the laminate 10.

### Support Layer and Projection Web

As the name implies, the support layer 14 is meant to support the projection web 16 containing the projections 12. The support layer 14 can be made from a number of structures provided the support layer 14 is capable of supporting the projection web 16. The primary functions of the support layer 14 are to protect the projection web 16 during the formation of the projections 12, to be able to bond to or be entangled with the projection web 16 and to aid in the further processing of the projection web 16 and the resultant fluid-entangled laminate web 10. Suitable materials for the support layer 14 can include, but are not limited to, nonwoven fabrics or webs, scrim materials, netting materials, paper/cellulose/wood pulp-based products which can be considered a subset of nonwoven fabrics or webs as well as foam materials, films and combinations of the foregoing provided the material or materials chosen are capable of withstanding the fluid-entangling process. A particularly well-suited material for the support layer 14 is a fibrous nonwoven web made from a plurality of randomly deposited fibers which may be staple length fibers such as are used, for example, in carded webs, air laid webs, etc., or they may be more continuous fibers such as are found in, for example, meltblown or spunbond webs. Due to the functions the support layer 14 must perform, the support layer 14 should have a higher degree of integrity than the projection web 16. In this regard, the support layer 14 should be able to remain substantially intact when it is subjected to the fluid-entangling process discussed in greater detail below. The degree of integrity of the support layer 14 should be such that the material forming the support layer 14 resists being driven down into and filling the hollow projections 12 of the projection web 16. As a result, when the support layer 14 is a fibrous nonwoven web, it is desirable that it should have a higher degree of fiber-to-fiber bonding and/or fiber entanglement than the fibers in the projection web 16. While it is desirable to have fibers from the support layer 14 entangle with the fibers of the projection web 16 adjacent the interface 27 between the two layers, it is generally desired that the fibers of this support layer 14 not be integrated or entangled into the projection web 16 to such a degree that large portions of these fibers find their way inside the hollow projections 12.

A function of the support layer 14 is to facilitate further processing of the projection web 16. Typically the fibers used to form the projection web 16 are more expensive than those used to form the support layer 14. As a result, it is desirable to keep the basis weight of the projection web 16 low. In so doing, however, it becomes difficult to process the projection web 16 subsequent to its formation. By attaching the projection web 16 to an underlying support layer 14, further processing, winding and unwinding, storage and other activities can be done more effectively.

In order to resist this higher degree of fiber movement, as mentioned above, it is desirable that the support layer 14 have a higher degree of integrity than the projection web 16. This higher degree of integrity can be brought about in a number of ways. One is fiber-to-fiber bonding which can be achieved through thermal or ultrasonic bonding of the fibers to one another with or without the use of pressure as in through air bonding, point bonding, powder bonding, chemical bonding, adhesive bonding, embossing, calender bonding, etc. In addition, other materials may be added to the fibrous mix such as adhesives and/or bicomponent fibers. Pre-entanglement of the fibrous nonwoven support layer 14 may also be used such as, for example, by subjecting the web to hydroentangling, needle punching, etc., prior to this web 14 being joined to the projection web 16. Combinations of the foregoing are also possible. Still other materials such as foams, scrims and nettings may have enough initial integrity so as to not need further processing. The level of integrity can in many cases be visually observed due to, for example, the observation with the unaided eye of such techniques as point bonding which is commonly used with fibrous nonwoven webs such as spunbond webs and staple fiber-containing webs. Further magnification of the support layer 14 may also reveal the use of fluid-entangling or the use of thermal and/or adhesive bonding to join the fibers together. Depending on whether samples of the individual layers (14 and 16) are available, tensile testing in either or both of the machine and cross-machine directions may be undertaken to compare the integrity of the support layer 14 to the projection web 16. See for example ASTM test D5035-11 which is incorporated herein in its entirety for all purposes.

The type, basis weight, strength and other properties of the support layer 14 can be chosen and varied depending upon the particular end use of the resultant laminate 10. When the laminate 10 is to be used as part of an absorbent article such as a personal care absorbent article, wipe, etc., it is generally desirable that the support layer 14 be a layer that is fluid pervious, has good wet and dry strength, is able to absorb fluids such as body exudates, possibly retain the fluids for a certain period of time and then release the fluids to one or more subjacent layers. In this regard, fibrous nonwovens such as spunbond webs, meltblown webs and carded webs such as airlaid webs, bonded carded webs and coform materials are particularly well suited as support layers 14. Foam materials and scrim materials are also well suited. In addition, the support layer 14 may be a multi-layered material due to the use of several layers or the use of multi-bank formation processes as are commonly used in making spunbond webs and meltblown webs as well as layered combinations of meltblown and spunbond webs. In the formation of such support layers 14, both natural and synthetic materials may be used alone or in combination to fabricate the material. Generally, for the end-use applications outlined herein, support layer 14 basis weights will range between about 5 and about 40 gsm though basis weights outside this range may be used depending upon the particular end-use application.

The type, basis weight and porosity of the support layer 14 will affect the process conditions necessary to form the projections 12 in the projection web 16. Heavier basis weight materials will increase the entangling force of the entangling fluid streams needed to form the projections 12 in the projection web 16. However, heavier basis weight support layers 14 will also provide improved support for the projection web 16, as a major problem with the projection web 16 by itself is that it is too stretchy to maintain the shape of the projections 12 post the formation process. The projection web 16 by itself unduly elongates in the machine direction due to the mechanical forces exerted on it by subsequent winding and converting processes which diminish and distort the projections 12. Also, without the support layer 14, the projections 12 in the projection web 16 collapse due to the winding pressures and compressive weights the projection web 16 experiences in the winding process and subsequent conversion and do not recover to the extent they do with the support layer 14.

The support layer 14 may be subjected to further treatment and/or additives to alter or enhance its properties. For example, surfactants and other chemicals may be added both internally and externally to the components forming all or a portion of the support layer 14 to alter or enhance its properties. Compounds commonly referred to as hydrogels or superabsorbents which absorb many times their weight in liquids may be added to the support layer 14 in both particulate and fiber form.

The projection web 16 is made from a plurality of randomly deposited fibers which may be staple length fibers such as those that are used, for example, in carded webs, airlaid webs, coform webs, etc., or they may be more continuous fibers such as those that are found in, for example, meltblown or spunbond webs. The fibers in the projection web 16 desirably should have less fiber-to-fiber bonding and/or fiber entanglement and thus less integrity as compared to the integrity of the support layer 14, especially when the support layer 14 is a fibrous nonwoven web. The fibers in the projection web 16 may have no initial fiber-to-fiber bonding for purposes of allowing the formation of the hollow projections 12 as will be explained in further detail below in connection with the description of one or more of the embodiments of the process and apparatus for forming the fluid-entangled laminate web 10. Alternatively, when both the support layer 14 and the projection web 16 are both fibrous nonwoven webs, the projection web 16 will have less integrity than the support layer 14 due to the projection web 16 having, for example, less fiber-to-fiber bonding, less adhesive or less pre-entanglement of the fibers forming the web 16.

The projection web 16 must have a sufficient amount of fiber movement capability to allow the below-described fluid entangling process to be able to move fibers of the projection web 16 out of the X-Y plane of the projection web 16, as shown in FIG. 1, and into the perpendicular or Z-direction (the direction of its thickness 28) of the web 16 so as to be able to form the hollow projections 12. If more continuous fiber structures are being used such as meltblown or spunbond webs, it is desirable to have little or no pre-bonding of the projection web 16 prior to the fluid-entanglement process. Longer fibers such as are generated in meltblowing and spunbonding processes (which are often referred to as continuous fibers to differentiate them from staple length fibers) will typically require more force to displace the fibers in the Z-direction than will shorter, staple length fibers that typically have fiber lengths less than 100 millimeters (mm) and more typically fiber lengths in the 10 to 60 mm range. Conversely, staple fiber webs such as carded webs and airlaid webs can have some degree of pre-bonding or entanglement of the fibers due to their shorter length. Such shorter fibers require less fluid force from the fluid-entangling streams to move them in the Z-direction to form the hollow projections 12. As a result, a balance must be met between fiber length, degree of pre-fiber bonding, fluid force, web speed and dwell time so as to be able to create the hollow projections 12 without, unless desired, forming apertures in the land areas 19, the hollow projections 12, or forcing too much material into the interior hollow space 21 of the projections 12 thereby making the projections 12 too rigid for some end-use applications.

Generally, the projection web 16 will have a basis weight ranging between about 10 and about 60 gsm for the uses outlined herein but basis weights outside this range may be used depending upon the particular end-use application. Spunbond webs will typically have basis weights of between about 15 and about 50 grams per square meter (gsm) when being used as the projection web 16. Fiber diameters will range between about 5 and about 20 microns. The fibers may be single component fibers formed from a single polymer composition or they may be bicomponent or multicomponent fibers wherein one portion of the fiber has a lower melting point than the other components so as to allow fiber-to-fiber bonding through the use of heat and/or pressure. Hollow fibers may also be used. The fibers may be formed from any polymer formulations typically used to form spunbond webs. Examples of such polymers include, but are not limited to, polypropylene (PP), polyester (PET), polyamide (PA), polyethylene (PE) and polylactic acid (PLA). The spunbond webs may be subjected to post-formation bonding and entangling techniques if necessary to improve the processability of the web prior to it being subjected to the projection forming process.

Meltblown webs will typically have basis weights of between about 20 and about 50 grams per square meter (gsm) when being used as the projection web 16. Fiber diameters will range between about 0.5 and about 5 microns. The fibers may be single component fibers formed from a single polymer composition or they may be bicomponent or multicomponent fibers wherein one portion of the fiber has a lower melting point than the other components so as to allow fiber-to-fiber bonding through the use of heat and/or pressure. The fibers may be formed from any polymer formulations typically used to form the aforementioned spunbond webs. Examples of such polymers include, but are not limited to, PP, PET, PA, PE and PLA.

Carded and airlaid webs use staple fibers that will typically range in length between about 10 and about 100 millimeters. Fiber denier will range between about 0.5 and about 6 denier depending upon the particular end use. Basis weights will range between about 20 and about 60 gsm. The staple fibers may be made from a wide variety of polymers including, but not limited to, PP, PET, PA, PE, PLA, cotton, rayon flax, wool, hemp and regenerated cellulose such as, for example, viscose. Blends of fibers may be utilized too such as blends of bicomponent fibers and single component fibers as well as blends of solid fibers and hollow fibers. If bonding is desired, it may be accomplished in a number of ways including, for example, through-air bonding, calender bonding, point bonding, chemical bonding and adhesive bonding such as powder bonding. If needed, to further enhance the integrity and processability of such webs prior to the projection forming process, they may be subjected to pre-entanglement processes to increase fiber entanglement within the projection web 16 prior to the formation of the projections 12. Hydroentangling is particularly advantageous in this regard.

While the foregoing nonwoven web types and formation processes are suitable for use in conjunction with the projection web 16, it is anticipated that other webs and formation processes may also be used provided the webs are capable of forming the hollow projections 12.

### Process Description

To form the materials according to the present invention, a fluid-entangling process must be employed. Any number of fluids may be used to join the support layer 14 and projection web 16 together, including both liquids and gases. The most common technology used in this regard is referred to as spunlace or hydroentangling technology which uses pressurized water as the fluid for entanglement.

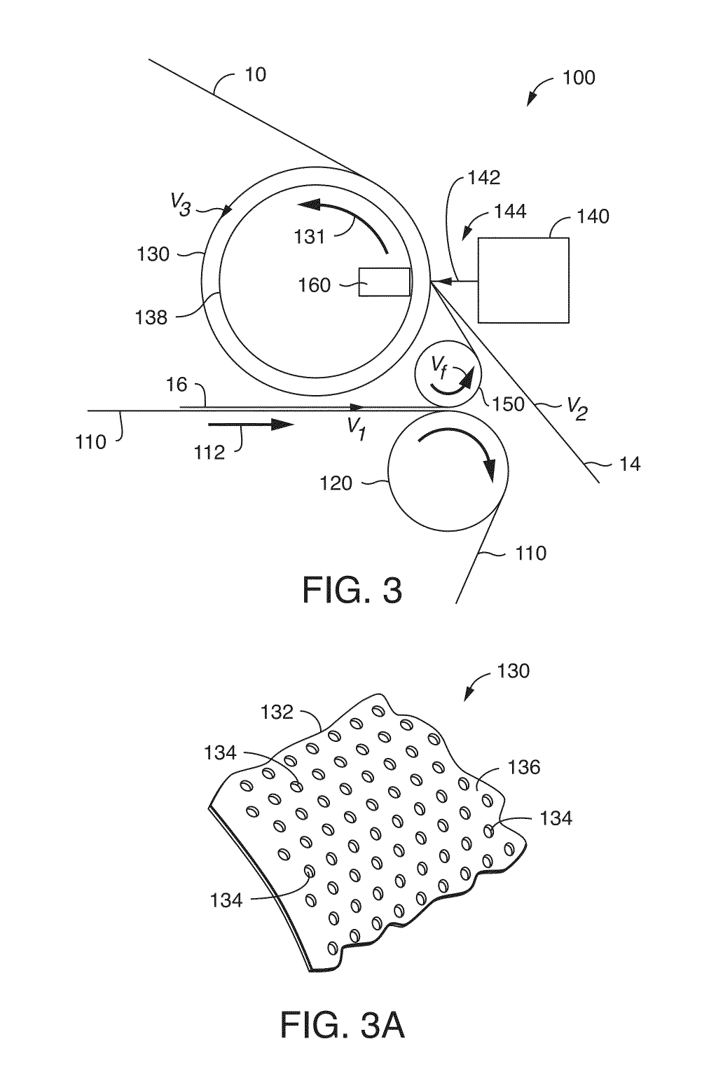

Referring to FIG. 3, there is shown a first embodiment of a process and apparatus 100 for forming a fluid-entangled laminate web 10 with hollow projections 12 according to the present invention. The apparatus 100 includes a first transport belt 110, a transport belt drive roll 120, a projection forming surface 130, a fluid entangling device 140, an optional overfeed roll 150, and a fluid removal system 160 such as a vacuum or other conventional suction device. Such vacuum devices and other means are well known to those of ordinary skill in the art. The transport belt 110 is used to carry the projection web 16 into the apparatus 100. If any pre-entangling is to be done on the projection web 16 upstream of the process shown in FIG. 3, the transport belt 110 may be porous. The transport belt 110 travels in a first direction (which is the machine direction) as shown by arrow 112 at a first speed or velocity V1. The transport belt 110 can be driven by the transport belt drive roller 120 or other suitable means as are well known to those of ordinary skill in the art.

The projection forming surface 130 as shown in FIG. 3 is in the form of a texturizing drum 130, a partially exploded view of the surface which is shown in FIG. 3A. The projection forming surface 130 moves in the machine direction as shown by arrow 131 in FIG. 3 at a speed or velocity V3. It is driven and its speed controlled by any suitable drive means (not shown) such as electric motors and gearing as are well known to those of ordinary skill in the art. The texturing drum 130 depicted in FIGS. 3 and 3A consists of a forming surface 132 containing a pattern of forming holes 134 that correspond to the shape and pattern of the desired projections 12 in the projection web 16. The forming holes 134 are separated by a land area 136. The forming holes 134 can be of any shape and any pattern. As can be seen from the Figures depicting the laminates 10 according to the present invention, the hole shapes are round but it should be understood that any number of shapes and combination of shapes can be used depending on the end use application. Examples of possible hole shapes include, but are not limited to, ovals, crosses, squares, rectangles, diamond shapes, hexagons and other polygons. Such shapes can be formed in the drum surface by casting, punching, stamping, laser-cutting and water-jet cutting. The spacing of the forming holes 134 and therefore the degree of land area 136 can also be varied depending upon the particular end application of the fluid-entangled laminate web 10. Further, the pattern of the forming holes 134 in the texturizing drum 130 can be varied depending upon the particular end application of the fluid-entangled laminate web 10. The material forming the texturizing drum 130 may be any number of suitable materials commonly used for such forming drums including, but not limited to, sheet metal, plastics and other polymer materials, rubber, etc. The forming holes 134 can be formed in a sheet of the material 132 that is then formed into a texturizing drum 130 or the texturizing drum 130 can be molded or cast from suitable materials or printed with 3D printing technology. Typically, the perforated drum 130 is removably fitted onto and over an optional porous inner drum shell 138 so that different forming surfaces 132 can be used for different end product designs. The porous inner drum shell 138 interfaces with the fluid removal system 160 which facilitates pulling the entangling fluid and fibers down into the forming holes 134 in the outer texturizing drum surface 132 thereby forming the hollow projections 12 in the projection web 16. The porous inner drum shell 138 also acts as a barrier to retard further fiber movement down into the fluid removal system 160 and other portions of the equipment thereby reducing fouling of the equipment. The porous inner drum shell 138 rotates in the same direction and at the same speed as the texturizing drum 130. In addition, to further control the height of the projections 12, the distance between the inner drum shell 138 and the texturizing drum 130 can be varied. Generally, the spacing between the inner surface of projection forming surface 130 and the outer surface of the inner drum shell 138 will range between about 0 and about 5 mm. Other ranges can be used depending on the particular end-use application and the desired features of the fluid-entangled laminate web 10.

The depth of the forming holes 134 in the texturizing drum 130 or other projection forming surface 130 can be between 1 mm and 10 mm but preferably between around 3 mm and 5 mm to produce projections 12 with the shape most useful in the expected common applications. The hole cross-section size may be between about 2 mm and 10 mm but it is preferably between 3 mm and 6 mm as measured along the major axis and the spacing of the forming holes 134 on a center-to-center basis can be between 3 mm and 10 mm but preferably between 4 mm and 7 mm. The pattern of the spacing between forming holes 134 may be varied and selected depending upon the particular end use. Some examples of patterns include, but are not limited to, aligned patterns of rows and/or columns, skewed patterns, hexagonal patterns, wavy patterns and patterns depicting pictures, figures and objects.

The cross-sectional dimensions of the forming holes 134 and their depth influence the cross-section and height of the projections 12 produced in the projection web 16. Generally, hole shapes with sharp or narrow corners at the leading edge of the forming holes 134 as viewed in the machine direction 131 should be avoided as they can sometimes impair the ability to safely remove the fluid-entangled laminate web 10 from the forming surface 132 without damage to the projections 12. In addition, the thickness/hole depth in the texturizing drum 130 will generally tend to correspond to the depth or height of the hollow projections 12. It should be noted, however, that each of the hole depth, spacing, size, shape and other parameters may be varied independently of one another and may be varied based upon the particular end use of the fluid-entangled laminate web 10 being formed.

The land areas 136 in the forming surface 132 of the texturizing drum 130 are typically solid so as to not pass the entangling fluid 142 emanating from the pressurized fluid jets contained in the fluid entangling devices 140, but in some instances it may be desirable to make the land areas 136 fluid permeable to further texturize the exposed surface of the projection web 16. Alternatively, select areas of the forming surface 132 of the texturizing drum 130 may be fluid pervious and other areas impervious. For example, a central zone (not shown) of the texturizing drum 130 may be fluid pervious while lateral regions (not shown) on either side of the central region may be fluid impervious. In addition, the land areas 136 in the forming surface 132 may have raised areas (not shown) formed in or attached thereto to form the optional dimples 23 and/or the apertures 25 in the projection web 16 and the fluid-entangled laminate web 10.

In the embodiment of the apparatus 100 shown in FIG. 3, the projection forming surface 130 is shown in the form of a texturizing drum. It should be appreciated however that other means may be used to create the projection forming surface 130. For example, a foraminous belt or wire (not shown) may be used, which includes forming holes 134 formed in the belt or wire at appropriate locations. Alternatively, flexible rubberized belts (not shown) which are impervious to the pressurized fluid-entangling streams save the forming holes 134 may be used. Such belts and wires are well known to those of ordinary skill in the art as are the means for driving and controlling the speed of such belts and wires. A texturizing drum 130 is more advantageous for formation of the fluid-entangled laminate web 10 according to the present invention because it can be made with land areas 136 which are smooth and impervious to the entangling fluid 142 and which do not leave a wire weave pattern on the outer surface 26 of the projection web 16 as wire belts tend to do.

An alternative to a forming surface 132 with a hole-depth defining the projection height is a forming surface 132 that is thinner than the desired projection height but which is spaced away from the porous inner drum shell 138 surface on which it is wrapped. The spacing between the texturizing drum 130 and porous inner drum shell 138 may be achieved by any means that preferably does not otherwise interfere with the process of forming the hollow projections 12 and withdrawing the entangling fluid from the equipment. For example, one means is a hard wire or filament that may be inserted between the outer texturizing drum 130 and the porous inner drum shell 138 as a spacer or wrapped around the inner porous drum shell 138 underneath the texturizing drum 130 to provide the appropriate spacing. A shell depth of the forming surface 132 of less than 2 mm can make it more difficult to remove the projection web 16 and the laminate 10 from the texturizing drum 130 because the fibrous material of the projection web 16 can expand or be moved by entangling fluid flow into the overhanging area beneath the texturizing drum 130 which in turn can distort the resultant fluid-entangled laminate web 10. It has been found, however, that by using a support layer 14 in conjunction with the projection web 16 as part of the formation process, distortion of the resultant two layer fluid-entangled laminate web 10 can be greatly reduced. Use of the support layer 14 generally facilitates cleaner removal of the fluid-entangled laminate web 10 because the less extensible, more dimensionally stable support layer 14 takes the load while the fluid-entangled laminate 10 is removed from the texturizing drum 130. The higher tension that can be applied to the support layer 14, compared to a single projection web 16, means that as the fluid-entangled laminate 10 moves away from the texturizing drum 130, the projections 12 can exit the forming holes 134 smoothly in a direction roughly perpendicular to the forming surface 132 and co-axially with the forming holes 134 in the texturizing drum 130. In addition, by using the support layer 14, processing speeds can be increased.

To form the projections 12 in the projection web 16 and to laminate the support layer 14 and the projection web 16 together, one or more fluid-entangling devices 140 are spaced above the projection forming surface 130. The most common technology used in this regard is referred to as spunlace or hydroentangling technology which uses pressurized water as the fluid for entanglement. As an unbonded or relatively unbonded web or webs are fed into a fluid-entangling device 140, a multitude of high pressure fluid jets (not shown) from one or more fluid entangling devices 140 move the fibers of the webs and the fluid turbulence causes the fibers to entangle. These fluid streams, which in this case are water, can cause the fibers to be further entangled within the individual webs. The streams can also cause fiber movement and entanglement at the interface 27 of two or more webs/layers thereby causing the webs/layers to become joined together. Still further, if the fibers in a web, such as the projection web 16, are loosely held together, they can be driven out of their X-Y plane and thus in the Z-direction (see FIGS. 1 and 2A) to form the projections 12 which are preferably hollow. Depending on the level of entanglement needed, one or a plurality of such fluid entangling devices 140 can be used.

In FIG. 3, a single fluid entangling device 140 is shown but in succeeding Figures where multiple devices 140 are used in various regions of the apparatus 100, they are labeled with letter designators such as 140a, 140b, 140c, 140d and 140e. When multiple devices are used, the entangling fluid pressure in each subsequent fluid-entangling device 140 is usually higher than the preceding one so that the energy imparted to the webs/layers increases and so the fiber entanglement within or between the webs/layers increases. This reduces disruption of the overall evenness of the areal density of the web/layer by the pressurized fluid jets while achieving the desired level of entanglement and hence bonding of the webs/layers and formation of the projections 12. The entangling fluid 142 of the fluid entangling devices 140 emanates from injectors via jet packs or strips (not shown) consisting of a row or rows of pressurized fluid jets with small apertures of a diameter usually between 0.08 and 0.15 mm and spacing of around 0.5 mm in the cross-machine direction. The pressure in the jets can be between about 5 bar and about 400 bar, but typically is less than 200 bar, except for heavy fluid-entangled laminate webs 10 and when fibrillation is required. Other jet sizes, spacings, numbers of jets and jet pressures can be used depending upon the particular end application. Such fluid entangling devices 140 are well known to those of ordinary skill in the art and are readily available from such manufactures as Fleissner of Germany and Andritz-Perfojet of France.

The fluid-entangling devices 140 will typically have the jet orifices positioned or spaced between about 5 millimeters and about 20 millimeters and more typically between about 5 and about 10 millimeters from the projection forming surface 130, though the actual spacing can vary depending on the basis weights of the materials being acted upon, the fluid pressure, the number of individual jets being used, the amount of vacuum being used via the fluid removal system 160 and the speed at which the equipment is being run.

In the embodiments shown in FIGS. 3 through 7, the fluid-entangling devices 140 are conventional hydroentangling devices, the construction and operation of which are well known to those of ordinary skill in the art. See for example U.S. Pat. No. 3,485,706 to Evans, the contents of which is incorporated herein by reference in its entirety for all purposes. Also see the description of the hydraulic entanglement equipment described by Honeycomb Systems, Inc., Biddeford, Me., in the article entitled “Rotary Hydraulic Entanglement of Nonwovens”, reprinted from INSIGHT '86 INTERNATIONAL ADVANCED FORMING/BONDING Conference, the contents of which is incorporated herein by reference in its entirety for all purposes.

Returning again to FIG. 3, the projection web 16 is fed into the apparatus and process 100 at a speed V1, the support layer 14 is fed into the apparatus and process 100 at a speed V2 and the fluid-entangled laminate web 10 exits the apparatus and process 100 at a speed V3 which is the speed of the projection forming surface 130 and can also be referred to as the projection forming surface speed. As will be explained in greater detail below, these speeds V1, V2, and V3 may be the same as one another or varied to change the formation process and the properties of the resultant fluid-entangled laminate web 10. Feeding both the projection web 16 and the support layer 14 into the process at the same speed (V1 and V2) will produce a fluid-entangled laminate web 10 according to the present invention with the desired hollow projections 12. Feeding both the projection web 16 and the support layer 14 into the process at the same speed, which is faster than the machine direction speed (V3) of the projection forming surface 130, will also form the desired hollow projections 12.

Also shown in FIG. 3, is an optional overfeed roll 150 which may be driven at a speed or rate Vf. The overfeed roll 150 may be run at the same speed as the speed V1 of the projection web 16 in which case Vf will equal V1, or it may be run at a faster rate to tension the projection web 16 upstream of the overfeed roll 150 when overfeed is desired. Overfeed occurs when one or both of the incoming webs/layers (16, 14) are fed onto the projection forming surface 130 at a greater speed than the projection forming surface speed of the projection forming surface 130. It has been found that improved projection formation in the projection web 16 can be affected by feeding the projection web 16 onto the projection forming surface 130 at a higher rate than the incoming speed V2 of the support layer 14. In addition, however, it has been discovered that improved properties and projection formation can be accomplished by varying the feed rates of the webs/layers (16, 14) and by also using the overfeed roll 150 just upstream of the texturizing drum 130 to supply a greater amount of fiber via the projection web 16 for subsequent movement by the entangling fluid 142 down into the forming holes 134 in the texturizing drum 130. In particular, by overfeeding the projection web 16 onto the texturizing drum 130, improved projection formation can be achieved including increased projection height.

In order to provide an excess of fiber so that the height of the projections 12 is maximized, the projection web 16 can be fed onto the texturizing drum 130 at a greater surface speed (V1) than the texturizing drum 130 is traveling (V3). Referring to FIG. 3, when overfeed is desired, the projection web 16 is fed onto the texturizing drum 130 at a speed V1 while the support layer 14 is fed in at a speed V2 and the texturizing drum 130 is traveling at a speed V3, which is slower than V1 and can be equal to V2. The overfeed percent or ratio, the ratio at which the projection web 16 is fed onto the texturizing drum 130, can be defined as OF=[(V1/V3)−1])×(100 where V1 is the input speed of the projection web 16 and V3 is the output speed of the resultant fluid-entangled laminate web 10 and the speed of the texturizing drum 130. (When the overfeed roll 150 is being used to increase the speed of the incoming material onto the texturizing drum 130, it should be noted that the speed V1 of the material after the overfeed roll 150 will be faster than the speed V1 upstream of the overfeed roll 150. In calculating the overfeed ratio, it is this faster speed V1 that should be used.) Good formation of the projections 12 has been found to occur when the overfeed ratio is between about 10 and about 50 percent. Note, too, that this overfeeding technique and ratio can be used with respect to not just the projection web 16 only but to a combination of the projection web 16 and the support layer 14 as they are collectively fed onto the projection forming surface 130.

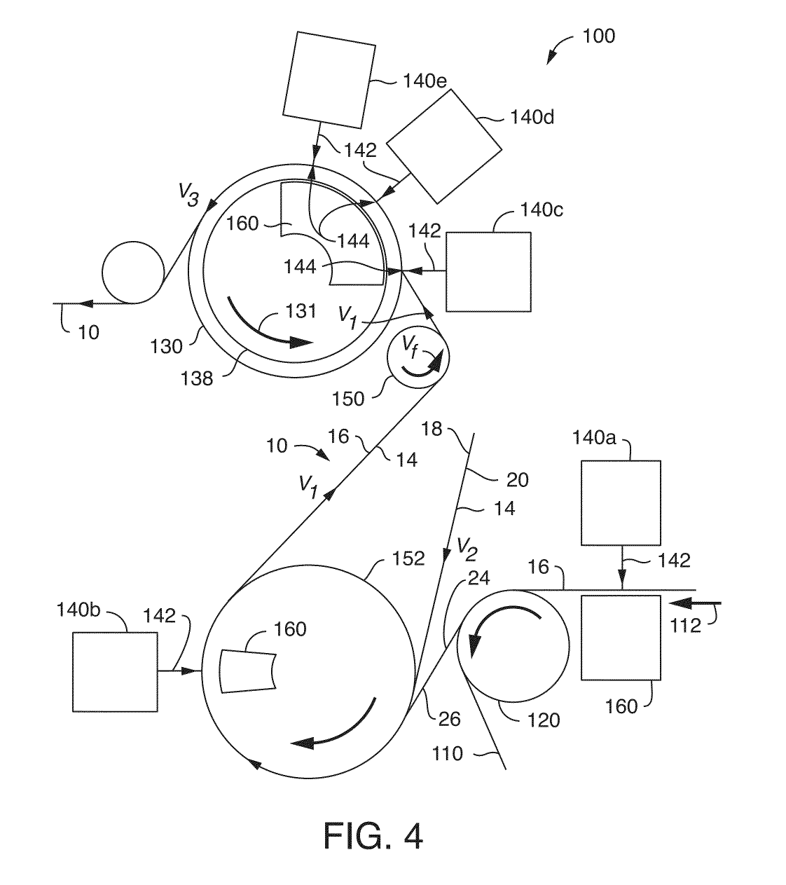

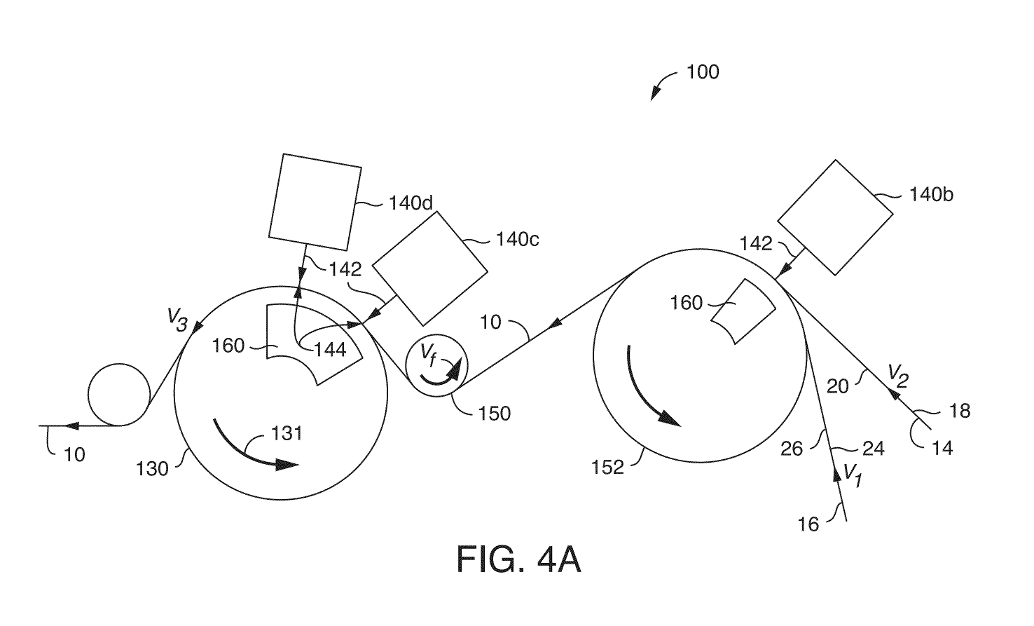

In order to minimize the length of projection web 16 that is supporting its own weight before being subjected to the entangling fluid 142 and to avoid wrinkling and folding of the projection web 16, the overfeed roll 150 can be used to carry the projection web 16 at speed V1 to a position close to the texturizing zone 144 on the texturizing drum 130. In the example illustrated in FIG. 3, the overfeed roll 150 is driven off the transport belt 110 but it is also possible to drive it separately so as to not put undue stress on the incoming projection web material 16. The support layer 14 may be fed into the texturizing zone 144 separately from the projection web 16 and at a speed V2 that may be greater than, equal to or less than the texturizing drum speed V3 and greater than, equal to or less than the projection web 16 speed V1. Preferably the support layer 14 is drawn into the texturizing zone 144 by its frictional engagement with the projection web 16 positioned on the texturizing drum 130 and so once on the texturizing drum 130, the support layer 14 has a surface speed close to the speed V3 of the texturizing drum 130 or it may be positively fed into the texturizing zone 144 at a speed close to the texturizing drum speed of V3. The texturizing process causes some contraction of the support layer 14 in the machine direction 131. The overfeed of either the support layer 14 or the projection web 16 can be adjusted according to the particular materials and the equipment and conditions being used so that the excess material that is fed into the texturizing zone 144 is used up, thereby avoiding any unsightly wrinkling in the resultant fluid-entangled laminate web 10. As a result, the two webs/layers (16, 14) will usually be under some tension at all times despite the overfeeding process. The take-off speed of the fluid-entangled laminate web 10 must be arranged to be close to the texturizing drum speed V3 such that excessive tension is not applied to the laminate in its removal from the texturizing drum 130 as such excessive tension would be detrimental to the clarity and size of the projections. An alternate embodiment of the process and apparatus 100 according to the present invention is shown in FIG. 4 in which like reference numerals are used for like elements. In this embodiment, the main differences relative to the process and apparatus shown in FIG. 3 are a pre-entanglement of the projection web 16 to improve its integrity prior to further processing via a pre-entanglement fluid entangling device 140a; a lamination of the projection web 16 to the support layer 14 via a lamination fluid entangling device 140b; and an increase in the number of fluid-entangling devices 140 (referred to as projection fluid entangling devices 140c, 140d and 140e) and thus an enlargement of the texturizing zone 144 on the texturizing drum 130 in the projection forming portion of the process.

The projection web 16 is supplied to the process/apparatus 100 via the transport belt 110. As the projection web 16 travels on the transport belt 110, it is subjected to a first fluid-entangling device 140a to improve the integrity of the projection web 16. This can be referred to as pre-entanglement of the projection web 16. As a result, this transport belt 110 should be fluid pervious to allow the entangling fluid 142 to pass through the projection web 16 and the transport belt 110. To remove the delivered entangling fluid 142, as in FIG. 3, a fluid removal system 160, such as a vacuum or other conventional fluid removal device, may be used below the transport belt 110. The fluid pressure from the first fluid entangling device 140a is generally in the range of about 10 to about 50 bar.

The support layer 14 and the projection web 16 are then fed to a lamination forming surface 152 with the first surface 18 of the support web or layer 14 facing and contacting the lamination forming surface 152 and the second surface 20 of the support layer 14 contacting the inner surface 24 of the projection web 16. (See FIGS. 2 and 4.) To entangle the support layer 14 and the projection web 16 together, one or more lamination fluid-entangling devices 140b are used in connection with the lamination forming surface 152 to affect fiber entanglement between the materials. Once again, a fluid removal system 160 is used to dispose of the entangling fluid 142. To distinguish the apparatus in this lamination portion of the overall apparatus and process 100 from the subsequent projection forming portion where the projections are formed, this equipment and process are referred to as lamination equipment as opposed to projection forming equipment. Thus, this portion is referred to as using a lamination forming surface 152 and a lamination fluid-entangling device 140b, which uses lamination fluid jets as opposed to projection forming jets. The lamination forming surface 152 is movable in the machine direction of the apparatus 100 at a lamination forming surface speed and should be permeable to the entangling fluid emanating from the lamination fluid jets located in the lamination fluid-entangling device 140b. The lamination fluid entangling device 140b has a plurality of lamination fluid jets which are capable of emitting a plurality of pressurized lamination fluid streams of entangling fluid 142 in a direction towards the lamination forming surface 152. The lamination forming surface 152, when in the configuration of a drum as shown in FIG. 4, can have a plurality of forming holes in its surface separated by land areas to make it fluid permeable or it can be made from conventional forming wire which is permeable as well. In this portion of the apparatus 100, complete bonding of the two materials (14 and 16) is not necessary. Process parameters for this portion of the equipment are similar to those for the projection forming portion and the description of the equipment and process in connection with FIG. 3. Thus, the speeds of the materials and surfaces in the lamination forming portion of the equipment and process may be varied as explained above with respect to the projection forming equipment and process described with respect to FIG. 3.

For example, the projection web 16 may be fed into the lamination forming process and onto the support layer 14 at a speed that is greater than the speed the support layer 14 is fed onto the lamination forming surface 152. Relative to entangling fluid pressures, lower lamination fluid jet pressures are desired in this portion of the equipment as additional entanglement of the webs/layers will occur during the projection forming portion of the process. As a result, lamination forming pressures from the lamination entangling device 140b will usually range between about 30 and about 100 bar.

When the plurality of lamination fluid streams 142 in the lamination fluid entangling device 140b are directed in a direction from the outer surface 26 of the projection web 16 towards the lamination forming surface 152, at least a portion of the fibers in the projection web 16 are caused to become entangled with support layer 14 to form a laminate web 10. Once the projection web 16 and support layer 14 are joined into a laminate 10, the laminate 10 leaves the lamination portion of the equipment and process (elements 140b and 152) and is fed into the projection forming portion of the equipment and process (elements 130, 140c, 140d, 140e and optional 150). As with the process shown in FIG. 3, the laminate 10 may be fed onto the projection forming surface/texturizing drum 130 at the same speed as the texturizing drum 130 is traveling, or it may be overfed onto the texturizing drum 130 using the overfeed roll 150 or by simply causing the laminate 10 to travel at a speed V1, which is greater than the speed V3 of the projection forming surface 130. As a result, the process variables described above with respect to FIG. 3 of the drawings may also be employed with the equipment and process shown in FIG. 4. In addition, as with the apparatus and materials in FIG. 3, if the overfeed roll 150 is used to increase the speed V1 of the laminate 10 as it comes in contact with the projection forming surface 130, it is this faster speed V1 after the overfeed roll 150 that should be used when calculating the overfeed ratio. The same approach should be used when calculating the overfeed ratio with the remainder of the embodiments shown in FIGS. 4a, 5, 6 and 7 if overfeed of material is being employed.

In the projection forming portion of the equipment, a plurality of pressurized projection fluid streams of entangling fluid 142 are directed from the projection fluid jets located in the projection fluid entangling devices (140c, 140d and 140e) into the laminate web 10 in a direction from the first surface 18 of the support layer 14 towards the projection forming surface 130 to cause a first plurality of the fibers of the projection web 16 in the vicinity of the forming holes 134 located in the projection forming surface 130 to be directed into the forming holes 134 to form the plurality of projections 12, which extend outwardly from the outer surface 26 of the projection web 16 thereby forming the fluid-entangled laminate web 10 according to the present invention. As with the other processes, the formed laminate web 10 is removed from the projection forming surface 130 and, if desired, may be subjected to the same or different further processing as described with respect to the process and apparatus in FIG. 3, such as drying to remove excess entangling fluid or further bonding or other steps. In the projection forming portion of the equipment and apparatus 100, forming pressures from the projection fluid entangling devices (140c, 140d and 140e) will usually range between about 80 and about 200 bar.

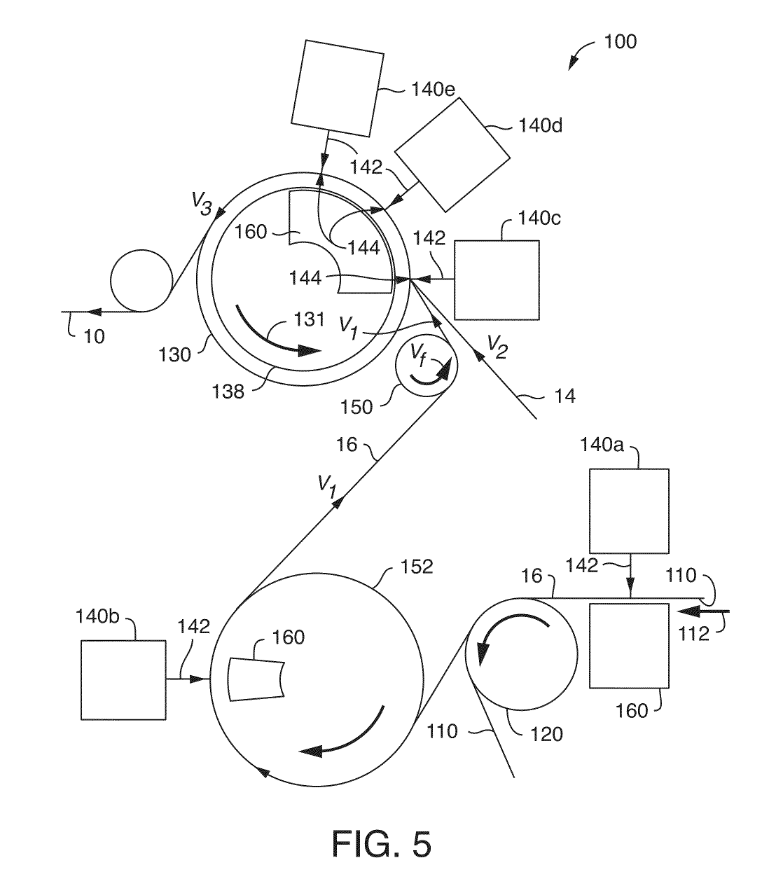

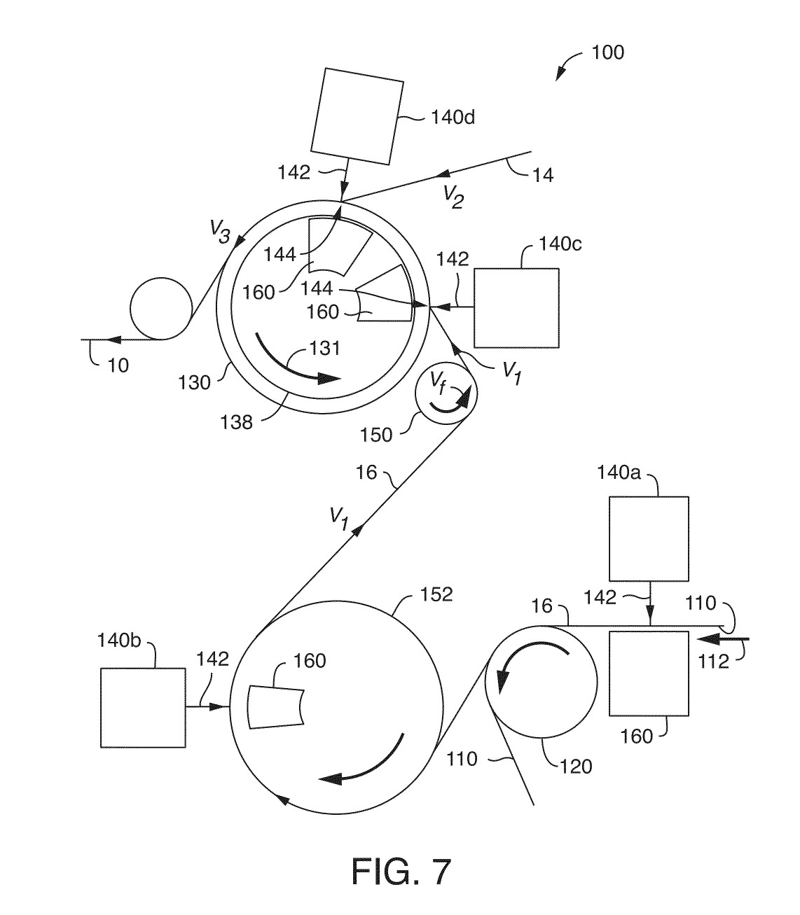

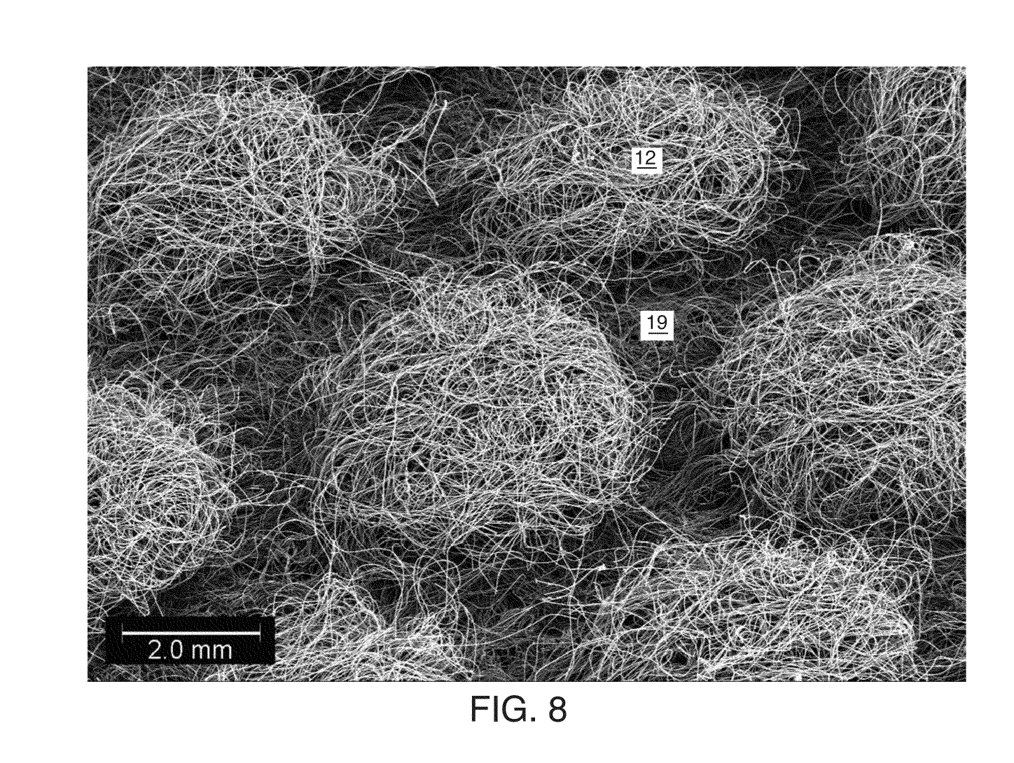

A further modification of the process and apparatus 100 of FIG. 4 is shown in FIG. 4A. In FIGS. 4, as well as subsequent embodiments of the apparatus and process shown in FIGS. 5 and 7, the fluid-entangled laminate web 10 is subjected to a pre-lamination step by way of the lamination forming surface 152 and a lamination fluid entangling device or devices 140b. In each of these configurations (FIGS. 4, 5 and 7), the material that is in direct contact with the lamination forming surface 152 is the first surface 18 of support layer 14. However, it is also possible to invert the support layer 14 and the projection web 16 such as is shown in FIG. 4A such that the outer surface 26 of the projection web 16 is the side that is in direct contact with the lamination forming surface 152, and this alternate version of the apparatus and process of FIGS. 4, 5 and 7 is also within the scope of the present invention as well as variations thereof.

Yet another alternate embodiment of the process and apparatus 100 according to the present invention is shown in FIG. 5. This embodiment is similar to that shown in FIG. 4 except that only the projection web 16 is subjected to pre-entanglement using the fluid entangling devices 140a and 140b prior to the projection web 16 being fed into the projection forming portion of the equipment. In addition, the support layer 14 is fed into the texturizing zone 144 on the projection forming surface/drum 130 in the same manner as in FIG. 3 though the texturizing zone 144 is supplied with multiple projection fluid entangling devices (140c, 140d and 140e).

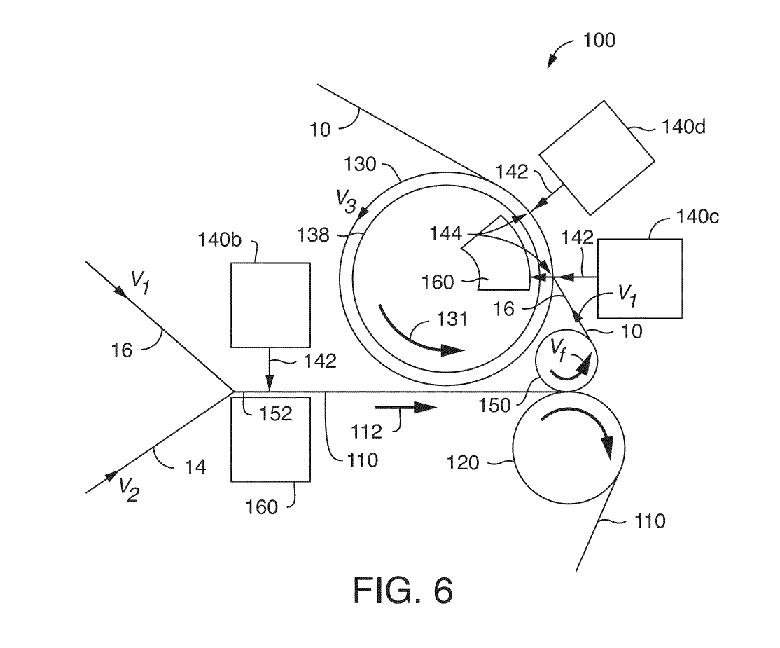

FIG. 6 depicts a further embodiment of the process and apparatus according to the present invention which, like FIG. 4, brings the projection web 16 and the support layer 14 into contact with one another for a lamination treatment in a lamination portion of the equipment and process utilizing a lamination forming surface 152 (which is the same element as the transport belt 110) and a lamination fluid entanglement device 140b. In addition, like the embodiment of FIG. 4, in the texturizing zone 144 of the projection forming portion of the process and apparatus 100, multiple projection fluid entangling devices (140c and 140d) are used.

FIG. 7 depicts a further embodiment of the process and apparatus 100 according to the present invention. In FIG. 7, the primary difference is that the projection web 16 undergoes a first treatment with entangling fluid 142 via a projection fluid entangling device 140c in the texturizing zone 144 before the second surface 20 of the support layer 14 is brought into contact with the inner surface 24 of the projection web 16 for fluid entanglement via the projection fluid entangling device 140d. In this manner, an initial formation of the projections 12 begins without the support layer 14 being in place. As a result, it may be desirable that the projection fluid-entangling device 140c be operated at a lower pressure than the projection fluid-entangling device 140d. For example, the projection fluid-entangling device 140c may be operated in a pressure range of about 100 to about 140 bar whereas the projection fluid entangling device 140d may be operated in a pressure range of about 140 to about 200 bar. Other combinations and ranges of pressures can be chosen depending upon the operating conditions of the equipment and the types and basis weights of the materials being used for the projection web 16 and the support layer 14.

In each of the embodiments of the process and apparatus 100, the fibers in the projection web 16 are sufficiently detached and mobile within the projection web 16 such that the entangling fluid 142 emanating from the projection fluid jets in the texturizing zone 144 is able to move a sufficient number of the fibers out of the X-Y plane of the projection web 16 in the vicinity of the forming holes 134 in the projection forming surface 130 and force the fibers down into the forming holes 134 thereby forming the hollow projections 12 in the projection web 16 of the fluid-entangled laminate web 10. In addition, by overfeeding at least the projection web 16 into the texturizing zone 144, enhanced projection formation can be achieved as shown by the below examples and photomicrographs.

### Product Embodiments

Fluid-entangled laminate webs according to the present invention have a wide variety of possible end uses especially where fluid adsorption, fluid transfer and fluid distancing are important. Two particularly though non-limiting areas of use involve food packaging and other absorbent articles such as personal care absorbent articles, bandages, and the like. In food packaging, it is desirable to use absorbent pads within the food packages to absorb fluids emanating from the packaged goods. This is particularly true with meat and seafood products. The bulky nature of the materials provided herein are beneficial in that the projections can help distance the packaged goods from the released fluids sitting in the bottom of the package. In addition, the laminate may be attached to a liquid impermeable material such as a film layer on the first side 18 of the support layer 14 via adhesives or other means so that fluids entering the laminate will be contained therein.

Personal care absorbent articles include such products as diapers, training pants, diaper pants, adult incontinence products, feminine hygiene products, wet and dry wipes, bandages, nursing pads, bed pads, changing pads, and the like. Feminine hygiene products include sanitary napkins, overnight pads, pantliners, tampons, and the like. When such products are used to absorb body fluids such as blood, urine, menses, feces, drainage fluids from injury and surgical sites, etc., commonly desired attributes of such products include fluid absorbency, softness, strength and separation from the affected body part to promote a cleaner, drier feel and to facilitate air flow for comfort and skin wellness. Laminates according to the present invention can be designed to provide such attributes. The hollow projections promote fluid transfer and separation from the remainder of the laminate. Because a lighter, softer material can be chosen for skin contact which in turn is supported by a stronger backing material, softness can also be imparted. In addition, because of the void volume created by the land areas surrounding the projections, area is provided to allow for the collection of unabsorbed solid materials. This void volume in turn can be useful when the product is removed as the combination of projections and void areas allow the laminate to be used in a cleaning mode to wipe and clean soiled skin surfaces. These same benefits can also be realized when the laminate is employed as either a wet or dry wipe which makes the laminate desirable for such products as baby and adult care wipes (wet and dry), household cleaning wipes, bath and beauty wipes, cosmetic wipes and applicators, etc. In addition, in any or all of these applications, the laminate web 10 and in particular the land areas 19 can be apertured to further facilitate fluid flow.

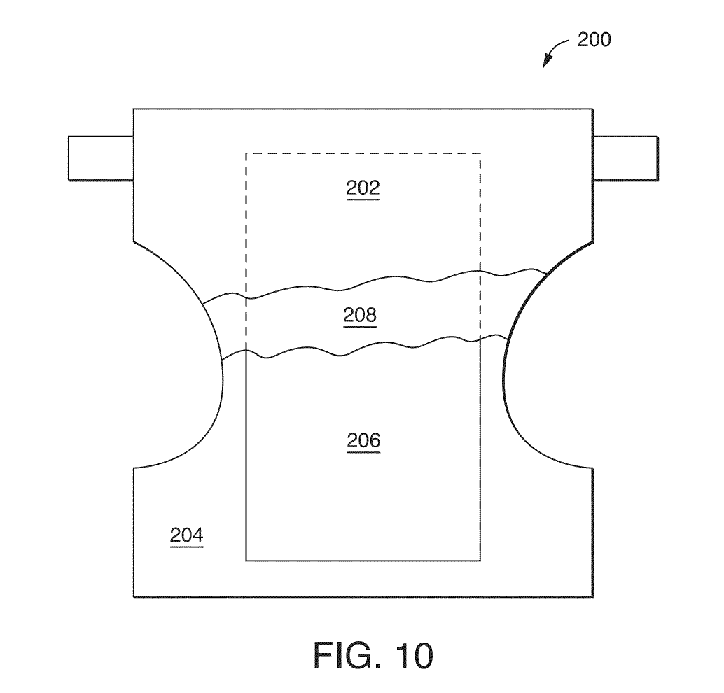

Personal care absorbent articles or simply absorbent articles typically have certain key components which may employ the laminates of the present invention. Turning to FIG. 10, there is shown an absorbent article 200 which in this case is a basic disposable diaper design. Typically, such products 200 will include a body side liner or skin-contacting material 202, a garment-facing material also referred to as a backsheet or baffle 204 and an absorbent core 206 disposed between the body side liner 202 and the backsheet 204. In addition, it is also very common for the product to have an optional layer 208 which is commonly referred to as a surge or transfer layer disposed between the body side liner 202 and the absorbent core 206. Other layers and components may also be incorporated into such products as will be readily appreciated by those of ordinary skill in such product formation.

The fluid-entangled laminate web 10 according to the present invention may be used as all or a portion of any one or all of these aforementioned components of such personal care products 200, including one of the external surfaces (202 or 204). For example, the laminate web 10 may be used as the body side liner 202 in which case it is more desirable for the projections 12 to be facing outwardly so as to be in a body contacting position in the product 200. The laminate web 10 may also be used as the surge or transfer layer 208 or as the absorbent core 206 or a portion of the absorbent core 206. Finally, from a softness and aesthetics standpoint, the laminate web 10 may be used as the outermost side of the backsheet 204 in which case it may be desirable to attach a liquid impervious film or other material to the first side 18 of the support layer 14.

The laminate web 10 may also be used to serve several functions within a personal care absorbent article 200 such as is shown in FIG. 10. For example, the projection web 16 may function as the body side liner 202 and the support layer 14 may function as the surge layer 208. In this regard, the materials in the examples with the “S” support layers are particularly advantageous in providing such functions. See Example 1 and Tables 2 and 3.

When such products are in the form of diapers and adult incontinence devices, they can also include what are termed “ears” located in the front and/or back waist regions at the lateral sides of the products. These ears are used to secure the product about the torso of the wearer, typically in conjunction with adhesive and/or mechanical hook and loop fastening systems. In certain applications, the male component, such as the hook component, of such fastening systems are connected to the distal ends of the ears and are attached to and engaged with the female component, what is referred to as a “frontal patch” or “tape landing zone,” located on the front waist portion of the product. The fluid-entangled laminate web according to the present invention may be used for all or a portion of any one or more of these components and products. Providing a fluid-entangled laminate web according to the present invention as a component of a mechanical fastening system can provide several benefits. A laminate having hollow projections can provide a softer feel to the user and/or wearer of the absorbent article and can enhance the tactile aesthetics of the absorbent article. Such fluid-entangled laminate webs as a female component of a mechanical fastening system can also have an improved engagement with the male, or hook, component of a mechanical fastening system. Such mechanical fastening systems employing the fluid-entangled laminate web of the present invention can demonstrate an improvement in the peel strength of the laminate web. The visual appearance of the hollow projections can also provide the perception of softness and breathability. The fibrous nonwoven with hollow projections can also have greater tensile strength and can therefore provide improved fastening benefits at lower basis weight. The tensile strength of such a fibrous nonwoven can allow for the fibrous nonwoven with hollow projections to undergo various manufacturing and converting processes while still maintaining structure and strength.

When such absorbent articles are in the form of a training pant, diaper pant, incontinent pant or other product which is designed to be pulled on and worn like underwear, such products will typically include what are termed “side panels” joining the front and back waist regions of the product. Such side panels can include both elastic and non-elastic portions and the fluid-entangled laminate webs of the present invention can be used as all or a portion of these side panels as well.

Consequently, such absorbent articles can have at least one layer, all or a portion of which, comprises the fluid entangled laminate web of the present invention.

Additional details regarding an absorbent article 200 and the use of the fluid-entangled laminate web 10 described herein as a female component, also referred to as “frontal patch,” of a mechanical fastening system can be found below and with reference to the Figures.

### Absorbent Article:

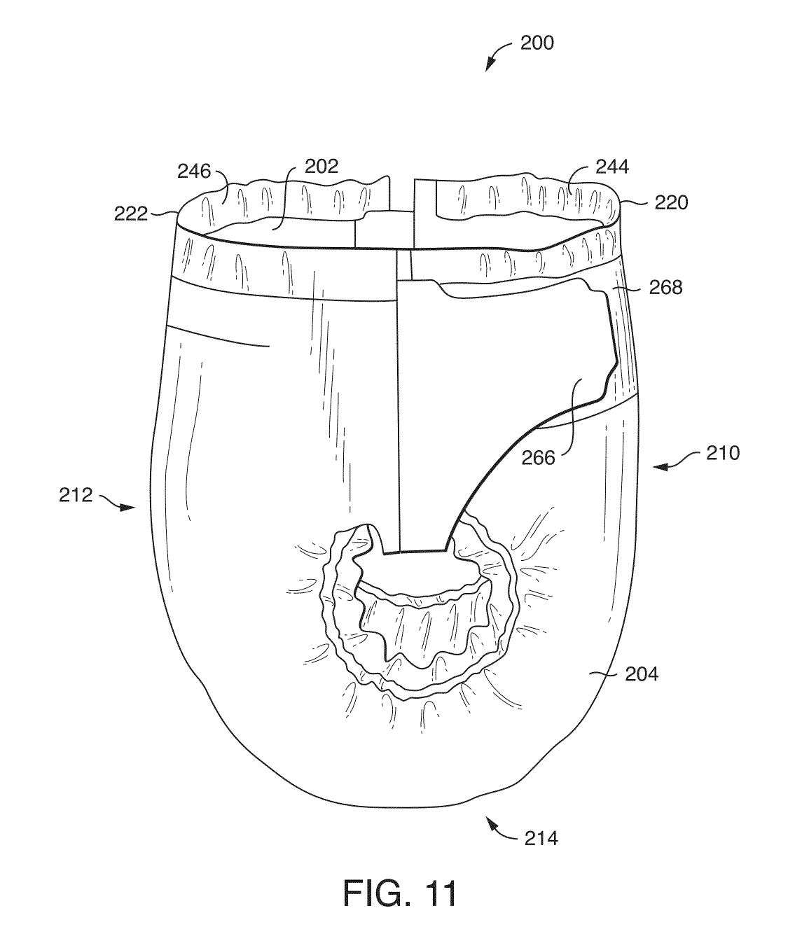

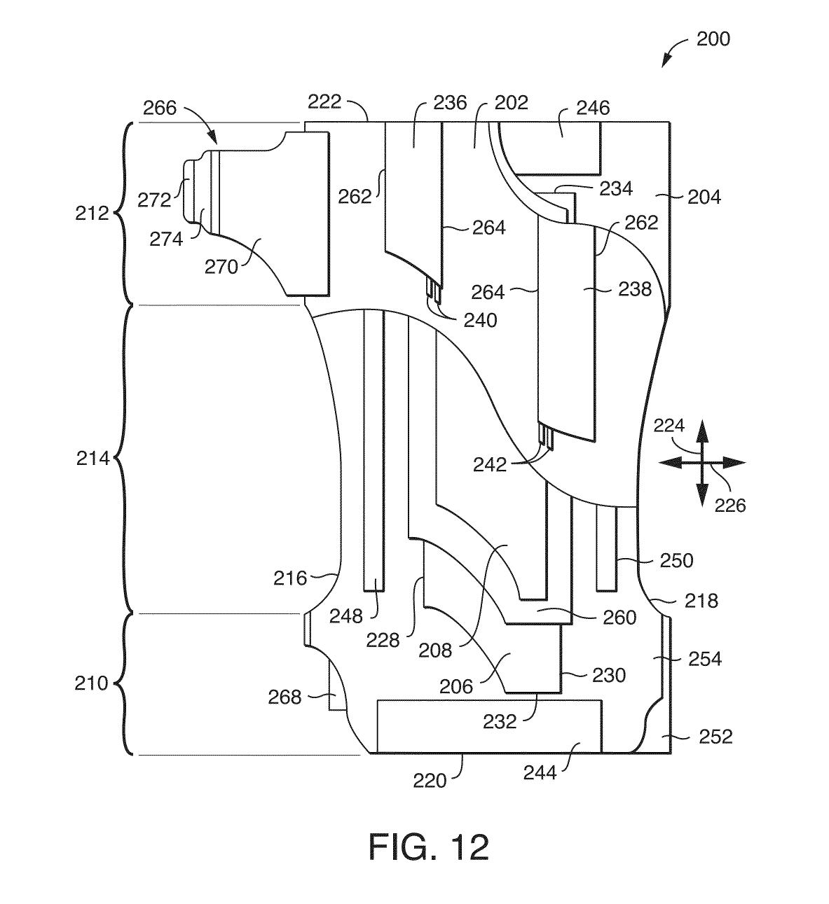

Referring to FIG. 11, a disposable absorbent article 200 of the present disclosure is exemplified in the form of a diaper. While the term “diaper” is utilized herein, it is to be understood that the disclosure herein can also apply to additional absorbent articles, such as, but not limited to, training pants, slip-on pants, youth pants, diaper pants, adult absorbent pants, and feminine care articles such as a wing or other attachment component. While the embodiments and illustrations described herein may generally apply to absorbent articles manufactured in the product longitudinal direction, which is hereinafter called the machine direction manufacturing of a product, it should be noted that one of ordinary skill could apply the information herein to absorbent articles manufactured in the latitudinal direction of the product which hereinafter is called the cross direction manufacturing of a product without departing from the spirit and scope of the disclosure. The absorbent article 200 illustrated in FIG. 11 includes a front waist region 210, back waist region 212, and a crotch region 214 interconnecting the front and back waist regions, 210 and 212, respectively. The absorbent article 200 has a pair of longitudinal side edges, 216 and 218 (shown in FIG. 12), and a pair of opposite waist edges, 220 and 222, respectively designated front waist edge 220 and back waist edge 222. The front waist region 210 can be contiguous with the front waist edge 220 and the back waist region 212 can be contiguous with the back waist edge 222.

Referring to FIG. 12, a non-limiting illustration of an absorbent article 200, such as, for example, a diaper, is illustrated in a top down view with portions cut away for clarity of illustration. The absorbent article 200 can include a backsheet 204 and a bodyside liner 202. In an embodiment, the bodyside liner 202 can be bonded to the backsheet 204 in a superposed relation by any suitable means such as, but not limited to, adhesives, ultrasonic bonds, thermal bonds, pressure bonds, or other conventional techniques. The backsheet 204 can define a length, or longitudinal direction 224, and a width, or lateral direction 226, which, in the illustrated embodiment, can coincide with the length and width of the absorbent article 200.

An absorbent core 206 can be disposed between the backsheet 204 and the bodyside liner 202. The absorbent core 206 can have longitudinal edges, 228 and 230, which, in an embodiment, can form portions of the longitudinal side edges, 216 and 218, respectively, of the absorbent article 200 and can have opposite end edges, 232 and 234, which, in an embodiment, can form portions of the waist edges, 220 and 222, respectively, of the absorbent article 200. In an embodiment, the absorbent core 206 can have a length and width that are the same as or less than the length and width of the absorbent article 200. In an embodiment, a pair of containment flaps, 236 and 238, can be present and can inhibit the lateral flow of body exudates.

The front waist region 210 can include the portion of the absorbent article 200 that, when worn, is positioned at least in part on the front of the wearer while the back waist region 212 can include the portion of the absorbent article 200 that, when worn, is positioned at least in part on the back of the wearer. The crotch region 214 of the absorbent article 200 can include the portion of the absorbent article 200, that, when worn, is positioned between the legs of the wearer and can partially cover the lower torso of the wearer. The waist edges, 220 and 222, of the absorbent article 200 are configured to encircle the waist of the wearer and together define the central waist opening. Portions of the longitudinal side edges, 216 and 218, in the crotch region 214 can generally define leg openings when the absorbent article 200 is worn.

The absorbent article 200 can be configured to contain and/or absorb liquid, solid, and semi-solid body exudates discharged from the wearer. For example, containment flaps, 236 and 238, can be configured to provide a bather to the lateral flow of body exudates. A flap elastic member, 240 and 242, can be operatively joined to each containment flap, 236 and 238, in any suitable manner known in the art. The elasticized containment flaps, 236 and 238, can define a partially unattached edge that can assume an upright configuration in at least the crotch region 214 of the absorbent article 200 to form a seal against the wearer's body. The containment flaps, 236 and 238, can be located along the absorbent article 200 longitudinal side edges, 216 and 218, and can extend longitudinally along the entire length of absorbent article 200 or can extend partially along the length of the absorbent article 200. Suitable construction and arrangements for containment flaps, 236 and 238, are generally well known to those skilled in the art and are described in U.S. Pat. No. 4,704,116 issued Nov. 3, 1987, to Enloe and U.S. Pat. No. 5,562,650 issued Oct. 8, 1996 to Everett et al., which are incorporated herein by reference.

To further enhance containment and/or absorption of body exudates, the absorbent article 200 can suitably include a front waist elastic member 244, a back waist elastic member 246, and leg elastic members, 248 and 250, as are known to those skilled in the art. The waist elastic members, 244 and 246, can be attached to the backsheet 204 and/or the bodyside liner 202 along the opposite waist edges, 220 and 222, and can extend over part or all of the waist edges, 220 and 222. The leg elastic members, 248 and 250, can be attached to the backsheet 204 and/or the bodyside liner 202 along the opposite longitudinal side edges, 216 and 218, and positioned in the crotch region 214 of the absorbent article 200.

The absorbent article 200 can further be provided with a mechanical fastening system. The mechanical fastening system can include one or more ears 266 which can include the male component of the mechanical fastening system, such as, for example, hooks. The mechanical fastening system can also include a female component 268, which is also referred to as a “frontal patch” 268. The female component 268 can be constructed of the fluid-entangled laminate web 10 described herein.

### Backsheet:

The backsheet 204 can be breathable and/or liquid impermeable. The backsheet 204 can be elastic, stretchable or non-stretchable. The backsheet 204 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the backsheet 204 can be constructed of a microporous polymeric film, such as polyethylene or polypropylene.

In an embodiment, the backsheet 204 can be a single layer of a liquid impermeable material. In an embodiment, the backsheet 204 can be suitably stretchable, and more suitably elastic, in at least the lateral or circumferential direction 226 of the absorbent article 200. In an embodiment, the backsheet 204 can be stretchable, and more suitably elastic, in both the lateral 226 and the longitudinal 224 directions. In an embodiment, the backsheet 204 can be a multi-layered laminate in which at least one of the layers is liquid impermeable. In an embodiment, the backsheet 204 may be a two layer construction, including an outer layer 252 material and an inner layer 254 material which can be bonded together such as by a laminate adhesive. Suitable laminate adhesives can be applied continuously or intermittently as beads, a spray, parallel swirls, or the like. Suitable adhesives can be obtained from Bostik Findlay Adhesives, Inc. of Wauwatosa, Wis., U.S.A. It is to be understood that the inner layer 254 can be bonded to the outer layer 252 utilizing ultrasonic bonds, thermal bonds, pressure bonds, or the like.

The outer layer 252 of the backsheet 204 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 252 of a backsheet 204 can be a 20 gsm spunbond polypropylene non-woven web. The outer layer 252 may also be constructed of the same materials from which the bodyside liner 202 can be constructed as described herein.

The liquid impermeable inner layer 254 of the backsheet 204 (or the liquid impermeable backsheet 204 where the backsheet 204 is of a single-layer construction) can be either vapor permeable (i.e., “breathable”) or vapor impermeable. The liquid impermeable inner layer 254 (or the liquid impermeable backsheet 204 where the backsheet 204 is of a single-layer construction) may be manufactured from a thin plastic film, although other liquid impermeable materials may also be used. The liquid impermeable inner layer 254 (or the liquid impermeable backsheet 204 where the backsheet 204 is of a single-layer construction) can inhibit liquid body exudates from leaking out of the absorbent article 200 and wetting articles, such as bed sheets and clothing, as well as the wearer and caregiver. An example of a material for a liquid impermeable inner layer 254 (or the liquid impermeable backsheet 204 where the backsheet 204 is of a single-layer construction) can be a printed 19 gsm Berry Plastics XP-8695H film or equivalent commercially available from Berry Plastics Corporation, Evansville, Ind., U.S.A.

Where the backsheet 204 is of a single layer construction, it can be embossed and/or matte finished to provide a more cloth-like texture or appearance. The backsheet 204 can permit vapors to escape from the absorbent article 200 while preventing liquids from passing through. A suitable liquid impermeable, vapor permeable material can be composed of a microporous polymer film or a non-woven material which has been coated or otherwise treated to impart a desired level of liquid impermeability.

### Absorbent Core:

The absorbent core 206 can be suitably constructed to be generally compressible, conformable, pliable, non-irritating to the wearer's skin and capable of absorbing and retaining liquid body exudates. The absorbent core 206 can be manufactured in a wide variety of sizes and shapes (for example, rectangular, trapezoidal, T-shape, I-shape, hourglass shape, etc.) and from a wide variety of materials. The size and the absorbent capacity of the absorbent core 206 should be compatible with the size of the intended wearer and the liquid loading imparted by the intended use of the absorbent article 200. Additionally, the size and the absorbent capacity of the absorbent core 206 can be varied to accommodate wearers ranging from infants to adults.

The absorbent core 206 may have a length ranging from about 150, 160, 170, 180, 190, 200, 210, 220, 225, 230, 240, 250, 260, 270, 280, 290, 300, 310, 320, 330, 340, or 350 mm to about 355, 360, 380, 385, 390, 395, 400, 410, 415, 420, 425, 440, 450, 460, 480, 500, 510, or 520 mm. The absorbent core 206 may have a crotch region 214 width ranging from about 30, 40, 50, 55, 60, 65, or 70 mm to about 75, 80, 85, 90, 95, 100, 105, 110, 115, 120, 125, 130, 140, 150, 160, 170 or 180 mm. The width of the absorbent core 206 located within the front waist region 210 and/or the back waist region 212 of the absorbent article 200 may range from about 50, 55, 60, 65, 70, 75, 80, 85, 90, or 95 mm to about 100, 105, 110, 115, 120, 125 or 130 mm. As noted herein, the absorbent core 206 can have a length and width that can be less than or equal to the length and width of the absorbent article 200.

In an embodiment, the absorbent article 200 can be a diaper having the following ranges of lengths and widths of an absorbent core 206 having an hourglass shape: the length of the absorbent core 206 may range from about 170, 180, 190, 200, 210, 220, 225, 240 or 250 mm to about 260, 280, 300, 310, 320, 330, 340, 350, 355, 360, 380, 385, or 390 mm; the width of the absorbent core 206 in the crotch region 214 may range from about 40, 50, 55, or 60 mm to about 65, 70, 75, or 80 mm; the width of the absorbent core 206 in the front waist region 210 and/or the back waist region 212 may range from about 80, 85, 90, or 95 mm to about 100, 105, or 110 mm.

In an embodiment, the absorbent article 200 may be a training pant or youth pant having the following ranges of lengths and widths of an absorbent core 206 having an hourglass shape: the length of the absorbent core 206 may range from about 400, 410, 420, 440 or 450 mm to about 460, 480, 500, 510 or 520 mm; the width of the absorbent core 206 in the crotch region 214 may range from about 50, 55, or 60 mm to about 65, 70, 75, or 80 mm; the width of the absorbent core 206 in the front waist region 210 and/or the back waist region 212 may range from about 80, 85, 90, or 95 mm to about 100, 105, 110, 115, 120, 125, or 130 mm.

In an embodiment, the absorbent article 200 can be an adult incontinence garment having the following ranges of lengths and widths of an absorbent core 206 having a rectangular shape: the length of the absorbent core 206 may range from about 400, 410 or 415 to about 425 or 450 mm; the width of the absorbent core 206 in the crotch region 214 may range from about 90, or 95 mm to about 100, 105, or 110 mm. It should be noted that the absorbent core 206 of an adult incontinence garment may or may not extend into either or both the front waist region 210 or the back waist region 212 of the absorbent article 200.

The absorbent core 206 can have two surfaces such as a wearer facing surface and a garment facing surface. Edges, such as longitudinal side edges, 228 and 230, and such as front and back end edges, 232 and 234, can connect the two surfaces.

In an embodiment, the absorbent core 206 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent core 206 can be a matrix of cellulosic fluff and superabsorbent material.

In an embodiment, the absorbent core 206 may be constructed of a single layer of materials, or in the alternative, may be constructed of two or more layers of materials. In an embodiment in which the absorbent core 206 has two layers, the absorbent core 206 can have a wearer facing layer suitably composed of hydrophilic fibers and a garment facing layer suitably composed at least in part of a high absorbency material commonly known as superabsorbent material. In such an embodiment, the wearer facing layer of the absorbent core 206 can be suitably composed of cellulosic fluff, such as wood pulp fluff, and the garment facing layer of the absorbent core 206 can be suitably composed of superabsorbent material, or a mixture of cellulosic fluff and superabsorbent material. As a result, the wearer facing layer can have a lower absorbent capacity per unit weight than the garment facing layer. The wearer facing layer may alternatively be composed of a mixture of hydrophilic fibers and superabsorbent material, as long as the concentration of superabsorbent material present in the wearer facing layer is lower than the concentration of superabsorbent material present in the garment facing layer so that the wearer facing layer can have a lower absorbent capacity per unit weight than the garment facing layer. It is also contemplated that, in an embodiment, the garment facing layer may be composed solely of superabsorbent material without departing from the scope of this disclosure. It is also contemplated that, in an embodiment, each of the layers, the wearer facing and garment facing layers, can have a superabsorbent material such that the absorbent capacities of the two superabsorbent materials can be different and can provide the absorbent core 206 with a lower absorbent capacity in the wearer facing layer than in the garment facing layer.

Various types of wettable, hydrophilic fibers can be used in the absorbent core 206. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. For example, one suitable type of fiber is a wood pulp that is a bleached, highly absorbent sulfate wood pulp containing primarily soft wood fibers. However, the wood pulp can be exchanged with other fiber materials, such as synthetic, polymeric, or meltblown fibers or with a combination of meltblown and natural fibers. In an embodiment, the cellulosic fluff can include a blend of wood pulp fluff. An example of wood pulp fluff can be “CoosAbsorb™ S Fluff Pulp” or equivalent, available from Abitibi Bowater, Greenville, S.C., U.S.A., which is a bleached, highly absorbent sulfate wood pulp containing primarily southern soft wood fibers.

The absorbent core 206 can be formed with a dry-forming technique, an air-forming technique, a wet-forming technique, a foam-forming technique, or the like, as well as combinations thereof. A coform nonwoven material may also be employed. Methods and apparatus for carrying out such techniques are well known in the art.

Suitable superabsorbent materials can be selected from natural, synthetic, and modified natural polymers and materials. The superabsorbent materials can be inorganic materials, such as silica gels, or organic compounds, such as cross-linked polymers. Cross-linking may be covalent, ionic, Van der Waals, or hydrogen bonding. Typically, a superabsorbent material can be capable of absorbing at least about ten times its weight in liquid. In an embodiment, the superabsorbent material can absorb more than twenty-four times its weight in liquid. Examples of superabsorbent materials include polyacrylamides, polyvinyl alcohol, ethylene maleic anhydride copolymers, polyvinyl ethers, hydroxypropyl cellulose, carboxymal methyl cellulose, polyvinylmorpholinone, polymers and copolymers of vinyl sulfonic acid, polyacrylates, polyacrylamides, polyvinyl pyrrolidone, and the like. Additional polymers suitable for superabsorbent material include hydrolyzed, acrylonitrile grafted starch, acrylic acid grafted starch, polyacrylates and isobutylene maleic anhydride copolymers and mixtures thereof. The superabsorbent material may be in the form of discrete particles. The discrete particles can be of any desired shape, for example, spiral or semi-spiral, cubic, rod-like, polyhedral, etc. Shapes having a largest greatest dimension/smallest dimension ratio, such as needles, flakes, and fibers are also contemplated for use herein. Conglomerates of particles of superabsorbent materials may also be used in the absorbent core 206.

In an embodiment, the absorbent core 206 can be free of superabsorbent material. In an embodiment, the absorbent core 206 can have at least about 15% by weight of a superabsorbent material. In an embodiment, the absorbent core 206 can have at least about 15, 20, 25, 30, 35, 40, 45, 50, 55, 60, 65, 70, 75, 80, 85, 90, 95, 99 or 100% by weight of a superabsorbent material. In an embodiment, the absorbent core 206 can have less than about 100, 99, 95, 90, 85, 80, 75, 70, 65, 60, 55, 50, 45, 40 35, 30, 25, or 20% by weight of a superabsorbent material. In an embodiment, the absorbent core 206 can have from about 15, 20, 25, 30, 35, 40, 45, 50, 55 or 60% to about 65, 70, 75, 80, 85, 90, 95, 99 or 100% by weight of a superabsorbent material. Examples of superabsorbent material include, but are not limited to, FAVOR SXM-9300 or equivalent available from Evonik Industries, Greensboro, N.C., U.S.A. and HYSORB 8760 or equivalent available from BASF Corporation, Charlotte, N.C., U.S.A.

The absorbent core 206 can be superposed over the inner layer 254 of the backsheet 204, extending laterally between the leg elastic members, 248 and 250, and can be bonded to the inner layer 254 of the backsheet 204, such as by being bonded thereto with adhesive. However, it is to be understood that the absorbent core 206 may be in contact with, and not bonded with, the backsheet 204 and remain within the scope of this disclosure. In an embodiment, the backsheet 204 can be composed of a single layer and the absorbent core 206 can be in contact with the singer layer of the backsheet 204. In an embodiment, a layer, such as but not limited to, a core wrap 260, can be positioned between the absorbent core 206 and the backsheet 204.

### Core Wrap:

In various embodiments an absorbent article 200 can be constructed without a core wrap 260. In various embodiments the absorbent article 200 can have a core wrap 260. In an embodiment, the core wrap 260 can be in contact with the absorbent core 206. In an embodiment, the core wrap 260 can be bonded to the absorbent core 206. Bonding of the core wrap 260 to the absorbent core 206 can occur via any means known to one of ordinary skill, such as, but not limited to, adhesives. In an embodiment, a core wrap 260 can be positioned between the bodyside liner 202 and the absorbent core 206. In an embodiment, a core wrap 260 can completely encompass the absorbent core 206 and can be sealed to itself. In such an embodiment, the core wrap 260 may be folded over on itself and then sealed using, for example, heat and/or pressure. In an embodiment, a core wrap 260 may be composed of separate sheets of material which can be utilized to partially or fully encompass the absorbent core 206 and which can be sealed together using a sealing means, such as an ultrasonic bonder or other thermochemical bonding means or the use of an adhesive.

In an embodiment, the core wrap 260 can be in contact with and/or bonded with the wearer facing surface of the absorbent core 206. In an embodiment, the core wrap 260 can be in contact with and/or bonded with the wearer facing surface and at least one of the edges, 228, 230, 232, or 234, of the absorbent core 206. In an embodiment, the core wrap 260 can be in contact with and/or bonded with the wearer facing surface, at least one of the edges, 228, 230, 232, or 234, and the garment facing surface of the absorbent core 206. In an embodiment, the absorbent core 206 may be partially or completely encompassed by a core wrap 260.

The core wrap 260 can be pliable, less hydrophilic than the absorbent core 206, and sufficiently porous to thereby permit liquid body exudates to penetrate through the core wrap 260 to reach the absorbent core 206. In an embodiment, the core wrap 260 can have sufficient structural integrity to withstand wetting thereof and of the absorbent core 206. In an embodiment, the core wrap 260 can be constructed from a single layer of material or it may be a laminate constructed from two or more layers of material.

In an embodiment, the core wrap 260 can include, but is not limited to, natural and synthetic fibers such as, but not limited to, polyester, polypropylene, acetate, nylon, polymeric materials, cellulosic materials such as wood pulp, cotton, rayon, viscose, LYOCELL® such as from Lenzing Company of Austria, or mixtures of these or other cellulosic fibers, and combinations thereof. Natural fibers can include, but are not limited to, wool, cotton, flax, hemp, and wood pulp. Wood pulps can include, but are not limited to, standard softwood fluffing grade such as “CoosAbsorb™ S Fluff Pulp” or equivalent available from Abitibi Bowater, Greenville, S.C., U.S.A., which is a bleached, highly absorbent sulfate wood pulp containing primarily southern soft wood fibers.

In various embodiments, the core wrap 260 can include cellulosic material. In various embodiments, the core wrap 260 can be creped wadding or a high-strength tissue. In various embodiments, the core wrap 260 can include polymeric material. In an embodiment, a core wrap 260 can include a spunbond material. In an embodiment, a core wrap 260 can include a meltblown material. In an embodiment, the core wrap 260 can be a laminate of a meltblown nonwoven material having fine fibers laminated to at least one spunbond nonwoven material layer having coarse fibers. In such an embodiment, the core wrap 260 can be a spunbond-meltblown (“SM”) material. In an embodiment, the core wrap 260 can be a spunbond-meltblown-spunbond (“SMS”) material. A non-limiting example of such a core wrap 260 can be a 10 gsm spunbond-meltblown-spunbond material. In various embodiments, the core wrap 260 can be composed of at least one material which has been hydraulically entangled into a nonwoven substrate. In various embodiments, the core wrap 260 can be composed of at least two materials which have been hydraulically entangled into a nonwoven substrate. In various embodiments, the core wrap 260 can have at least three materials which have been hydraulically entangled into a nonwoven substrate. A non-limiting example of a core wrap 260 can be a 33 gsm hydraulically entangled substrate. In such an example, the core wrap 260 can be a 33 gsm hydraulically entangled substrate composed of a 12 gsm spunbond material, a 10 gsm wood pulp material having a length from about 0.6 cm to about 5.5 cm, and an 11 gsm polyester staple fiber material. To manufacture the core wrap 260 just described, the 12 gsm spunbond material can provide a base layer while the 10 gsm wood pulp material and the 11 gsm polyester staple fiber material can be homogeneously mixed together and deposited onto the spunbond material and then hydraulically entangled with the spunbond material.

In various embodiments, a wet strength agent can be included in the core wrap 260. A non-limiting example of a wet strength agent can be Kymene 6500 (557LK) or equivalent, available from Ashland Inc. of Ashland, Ky., U.S.A. In various embodiments, a surfactant can be included in the core wrap 260. In various embodiments, the core wrap 260 can be hydrophilic. In various embodiments, the core wrap 260 can be hydrophobic and can be treated in any manner known in the art to be made hydrophilic.

In an embodiment, the core wrap 260 can be in contact with and/or bonded with an absorbent core 206 which is made at least partially of particulate material such as superabsorbent material. In an embodiment in which the core wrap 260 at least partially or completely encompasses the absorbent core 206, the core wrap 260 should not unduly expand or stretch as this might cause the particulate material to escape from the absorbent core 206. In an embodiment, the core wrap 260, while in a dry state, should have respective extension values at peak load in the machine and cross directions of 30 percent or less and 40 percent or less, respectively.

In an embodiment, the core wrap 260 may have a longitudinal length the same as, greater than, or less than the longitudinal length of the absorbent core 206. The core wrap 260 can have a longitudinal length ranging from about 150, 160, 170, 180, 190, 200, 210, 220, 225, 230, 240, 250, 260, 270, 280, 290, 300, 310, 320, 330, 340, or 350 mm to about 355, 360, 380, 385, 390, 395, 400, 410, 415, 420, 425, 440, 450, 460, 480, 500, 510, or 520 mm.

### Surge Layer:

In various embodiments the absorbent article 200 can have a surge layer 208. The surge layer 208 can help decelerate and diffuse surges or gushes of liquid body exudates penetrating the bodyside liner 202. In an embodiment, the surge layer 208 can be positioned between the bodyside liner 202 and the absorbent core 206 to take in and distribute body exudates for absorption by the absorbent core 206. In an embodiment, the surge layer 208 can be positioned between the bodyside liner 202 and a core wrap 260 if a core wrap 260 is present.

In an embodiment, the surge layer 208 can be in contact with and/or bonded with the bodyside liner 202. In an embodiment in which the surge layer 208 is bonded with the bodyside liner 202, bonding of the surge layer 208 to the bodyside liner 202 can occur through the use of an adhesive and/or point fusion bonding. The point fusion bonding can be selected from, but is not limited to, ultrasonic bonding, pressure bonding, thermal bonding, and combinations thereof. In an embodiment, the point fusion bonding can be provided in any pattern as deemed suitable.

The surge layer 208 may have any longitudinal length dimension as deemed suitable. The surge layer 208 may have a longitudinal length from about 120, 130, 140, 150, 160, 170, 180, 190, 200, 210, 220, 225, 230, 240, or 250 mm to about 260, 270, 280, 290, 300, 310, 320, 340, 350, 360, 380, 400, 410, 415, 420, 425, 440, 450, 460, 480, 500, 510 or 520 mm. In an embodiment, the surge layer 208 can have any length such that the surge layer 208 can be coterminous with the waist edges, 220 and 222, of the absorbent article 200.

In an embodiment, the longitudinal length of the surge layer 208 can be the same as the longitudinal length of the absorbent core 206. In such an embodiment the midpoint of the longitudinal length of the surge layer 208 can substantially align with the midpoint of the longitudinal length of the absorbent core 206.

In an embodiment, the longitudinal length of the surge layer 208 can be shorter than the longitudinal length of the absorbent core 206. In such an embodiment, the surge layer 208 may be positioned at any desired location along the longitudinal length of the absorbent core 206. As an example of such an embodiment, the absorbent article 200 may contain a target area where repeated liquid surges typically occur in the absorbent article 200. The particular location of a target area can vary depending on the age and gender of the wearer of the absorbent article 200. For example, males tend to urinate further toward the front region of the absorbent article 200 and the target area may be phased forward within the absorbent article 200. For example, the target area for a male wearer may be positioned about 2¾″ forward of the longitudinal midpoint of the absorbent core 206 and may have a length of about ±3″ and a width of about ±2″. The female target area can be located closer to the center of the crotch region 214 of the absorbent article 200. For example, the target area for a female wearer may be positioned about 1″ forward of the longitudinal midpoint of the absorbent core 206 and may have a length of about ±3″ and a width of about ±2″. As a result, the relative longitudinal placement of the surge layer 208 within the absorbent article 200 can be selected to best correspond with the target area of either or both categories of wearers.

In an embodiment, the absorbent article 200 may contain a target area centered within the crotch region 214 of the absorbent article 200 with the premise that the absorbent article 200 would be worn by a female wearer. The surge layer 208, therefore, may be positioned along the longitudinal length of the absorbent article 200 such that the surge layer 208 can be substantially aligned with the target area of the absorbent article 200 intended for a female wearer. Alternatively, the absorbent article 200 may contain a target area positioned between the crotch region 214 and the front waist region 210 of the absorbent article 200 with the premise that the absorbent article 200 would be worn by a male wearer. The surge layer 208, therefore, may be positioned along the longitudinal length of the absorbent article 200 such that the surge layer 208 can be substantially aligned with the target area of the absorbent article 200 intended for a male wearer.

In an embodiment, the surge layer 208 can have a size dimension that is the same size dimension as the target area of the absorbent article 200 or a size dimension greater than the size dimension of the target area of the absorbent article 200. In an embodiment, the surge layer 208 can be in contact with and/or bonded with the bodyside liner 202 at least partially in the target area of the absorbent article 200.

In various embodiments, the surge layer 208 can have a longitudinal length shorter than, the same as or longer than the longitudinal length of the absorbent core 206. In an embodiment in which the absorbent article 200 is a diaper, the surge layer 208 may have a longitudinal length from about 120, 130, 140, 150, 160, 170, or 180 mm to about 200, 210, 220, 225, 240, 260, 280, 300, 310 or 320 mm. In such an embodiment, the surge layer 208 may be shorter in longitudinal length than the longitudinal length of the absorbent core 206 and may be phased from the front end edge 232 of the absorbent core 206 a distance of from about 15, 20, or 25 mm to about 30, 35 or 40 mm. In an embodiment in which the absorbent article 200 may be a training pant or youth pant, the surge layer 208 may have a longitudinal length from about 120, 130, 140, 150, 200, 210, 220, 230, 240 or 250 mm to about 260, 270, 280, 290, 300, 340, 360, 400, 410, 420, 440, 450, 460, 480, 500, 510 or 520 mm. In such an embodiment, the surge layer 208 may have a longitudinal length shorter than the longitudinal length of the absorbent core 206 and may be phased a distance of from about 25, 30, or 40 mm to about 45, 50, 55, 60, 65, 70, 75, 80 or 85 mm from the front end edge 232 of the absorbent core 206. In an embodiment in which the absorbent article 200 is an adult incontinence garment, the surge layer 208 may have a longitudinal length from about 200, 210, 220, 230, 240, or 250 mm to about 260, 270, 280, 290, 300, 320, 340, 360, 380, 400, 410, 415, 425, or 450 mm. In such an embodiment, the surge layer 208 may have a longitudinal length shorter than the longitudinal length of the absorbent core 206 and the surge layer 208 may be phased a distance of from about 20, 25, 30 or 35 mm to about 40, 45, 50, 55, 60, 65, 70 or 75 mm from the front end edge 232 of the absorbent core 206.

The surge layer 208 may have any width as desired. The surge layer 208 may have a width dimension from about 15, 20, 25, 30, 35, 40, 45, 50, 55, 60, or 70 mm to about 80, 90, 100, 110, 115, 120, 130, 140, 150, 160, 170, or 180 mm. The width of the surge layer 208 may vary dependent upon the size and shape of the absorbent article 200 within which the surge layer 208 will be placed. The surge layer 208 can have a width smaller than, the same as, or larger than the width of the absorbent core 206. Within the crotch region 214 of the absorbent article 200, the surge layer 208 can have a width smaller than, the same as, or larger than the width of the absorbent core 206.

In an embodiment, the surge layer 208 can include natural fibers, synthetic fibers, superabsorbent material, woven material, nonwoven material, wet-laid fibrous webs, a substantially unbounded airlaid fibrous web, an operatively bonded, stabilized-airlaid fibrous web, or the like, as well as combinations thereof. In an embodiment, the surge layer 208 can be formed from a material that is substantially hydrophobic, such as a nonwoven web composed of polypropylene, polyethylene, polyester, and the like, and combinations thereof.

### Bodyside Liner:

In various embodiments, the bodyside liner 202 of the absorbent article 200 can overlay the absorbent core 206 and the backsheet 204 and can isolate the wearer's skin from liquid waste retained by the absorbent core 206. In various embodiments, a core wrap 260 can be positioned between the bodyside liner 202 and the absorbent core 206. In various embodiments, a surge layer 208 can be positioned between the bodyside liner 202 and the absorbent core 206 or a core wrap 260, if present. In various embodiments, the bodyside liner 202 can be bonded to the surge layer 208, or the core wrap 260 if no surge layer 208 is present, via adhesive and/or by a point fusion bonding. The point fusion bonding may be selected from ultrasonic, thermal, pressure bonding, and combinations thereof.

In an embodiment, the bodyside liner 202 can extend beyond the absorbent core 206 and/or a core wrap 260, and/or a surge layer 208 to overlay a portion of the backsheet 204 and can be bonded thereto by any method deemed suitable, such as, for example, by being bonded thereto by adhesive, to substantially enclose the absorbent core 206 between the backsheet 204 and the bodyside liner 202. The bodyside liner 202 may be narrower than the backsheet 204, but it is to be understood that the bodyside liner 202 and the backsheet 204 may be of the same dimensions. It is also contemplated that the bodyside liner 202 may not extend beyond the absorbent core 206 and/or may not be secured to the backsheet 204. The bodyside liner 202 can be suitably compliant, soft feeling, and non-irritating to the wearer's skin and can be the same as or less hydrophilic than the absorbent core 206 to permit body exudates to readily penetrate through to the absorbent core 206 and provide a relatively dry surface to the wearer.

The bodyside liner 202 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 202. The bodyside liner 202 can include a woven fabric, a nonwoven fabric, a polymer film, a film-fabric laminate, or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof.

For example, the bodyside liner 202 can be composed of a meltblown or spunbond web of polyolefin fibers. Alternatively, the bodyside liner 202 can be a bonded-carded web composed of natural and/or synthetic fibers. The bodyside liner 202 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating, or the like. The surfactant can be applied to the entire bodyside liner 202 or it can be selectively applied to particular sections of the bodyside liner 202.

In an embodiment, a bodyside liner 202 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 202 can be a spunbond substrate with a basis weight from about 10 or 12 to about 15 or 20 gsm. In an embodiment, a bodyside liner 202 can be a 12 gsm spunbond-meltblown-spunbond substrate having 10% meltblown content applied between the two spunbond layers.

Although the backsheet 204 and bodyside liner 202 can include elastomeric materials, it is contemplated that the backsheet 204 and the bodyside liner 202 can be composed of materials which are generally non-elastomeric. In an embodiment, the bodyside liner 202 can be stretchable, and more suitably elastic. In an embodiment, the bodyside liner 202 can be suitably stretchable and more suitably elastic in at least the lateral or circumferential direction of the absorbent article 200.

In other aspects, the bodyside liner 202 can be stretchable, and more suitably elastic, in both the lateral and the longitudinal directions.

### Containment Flaps:

In an embodiment, containment flaps, 236 and 238, can be secured to the bodyside liner 202 of the absorbent article 200 in a generally parallel, spaced relation with each other laterally inward of the longitudinal side edges, 216 and 218, to provide a bather against the flow of body exudates to the leg openings. In an embodiment, the containment flaps, 236 and 238, can extend longitudinally from the front waist region 210 of the absorbent article 200, through the crotch region 214 to the back waist region 212 of the absorbent article 200. The containment flaps, 236 and 238, can be bonded to the bodyside liner 202 by a seam of adhesive to define a fixed proximal end 262 of the containment flaps, 236 and 238.

The containment flaps, 236 and 238, can be constructed of a fibrous material which can be similar to the material forming the bodyside liner 202. Other conventional material, such as polymer films, can also be employed. Each containment flap, 236 and 238, can have a moveable distal end 264 which can include flap elastics, such as flap elastics 240 and 242, respectively. Suitable elastic materials for the flap elastic, 240 and 242, can include sheets, strands or ribbons of natural rubber, synthetic rubber, or thermoplastic elastomeric materials.

The flap elastics, 240 and 242, as illustrated, can have two strands of elastomeric material extending longitudinally along the distal ends 264 of the containment flaps, 236 and 238, in generally parallel, spaced relation with each other. The elastic strands can be within the containment flaps, 236 and 238, while in an elastically contractible condition such that contraction of the strands gathers and shortens the distal ends 264 of the containment flaps, 236 and 238. As a result, the elastic strands can bias the distal ends 264 of each containment flap, 236 and 238, toward a position spaced from the proximal end 262 of the containment flaps, 236 and 238, so that the containment flaps, 236 and 238, can extend away from the bodyside liner 202 in a generally upright orientation of the containment flaps, 236 and 238, especially in the crotch region 214 of the absorbent article 200, when the absorbent article 200 is fitted on the wearer. The distal end 264 of the containment flaps, 236 and 238, can be connected to the flap elastics, 240 and 242, by partially doubling the containment flap, 236 and 238, material back upon itself by an amount which can be sufficient to enclose the flap elastics, 240 and 242. It is to be understood, however, that the containment flaps, 236 and 238, can have any number of strands of elastomeric material and may also be omitted from the absorbent article 200 without departing from the scope of this disclosure.

### Leg Elastics:

Leg elastic members, 248 and 250, can be secured to the backsheet 204, such as by being bonded thereto by laminate adhesive, generally laterally inward of the longitudinal side edges, 216 and 218, of the absorbent article 200. In an embodiment, the leg elastic members, 248 and 250, may be disposed between the inner layer 254 and outer layer 252 of the backsheet 204 or between other layers of the absorbent article 200. A wide variety of elastic materials may be used for the leg elastic members, 248 and 250. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate.

### Mechanical Fastening System:

In an embodiment, the absorbent article 200 can include a mechanical fastening system. The mechanical fastening system can include one or more ears 266 which can include the male component of the mechanical fastening system, such as, for example, hooks. The mechanical fastening system can also include the female component 268, which can also be referred to herein as a “frontal patch” 268. The female component 268 can be constructed of the fluid-entangled laminate web 10 described herein. Portions of the mechanical fastening system may be included in the front waist region 210, back waist region 212, or both. The mechanical fastening system can be configured to secure the absorbent article 200 about the waist of the wearer and maintain the absorbent article 200 in place during use.

In an embodiment, each ear 266 can extend laterally at the opposed, lateral ends of at least one of the waist regions, 210 or 212, of the absorbent article 200. In an embodiment, each ear 266 can substantially span from a laterally extending, terminal waist edge, such as waist edges 220 and 222, to approximately the location of its associated and corresponding leg opening of the absorbent article 200.

In an embodiment, the ears 266 can be integrally formed with the absorbent article 200. In an embodiment, the ears 266 can be integrally formed from the material constructing the backsheet 204 or may be integrally formed from the material constructing the bodyside liner 202. In an embodiment, the ears 266 can be provided by one or more separately provided members that are connected and assembled to the backsheet 204, to the bodyside liner 202, in-between the backsheet 204 and the bodyside liner 202, or in various fixedly bonded combinations of such assemblies.

In an embodiment, each ear 266 can be formed from a separately provided material or laminate of materials which can then be suitably assembled and bonded to the selected front and/or rear waist region, 210 and/or 212, respectively, of the absorbent article 200. In an embodiment, each ear 266 can be bonded to the backsheet 204 in the rear waist region 212 along an ear attachment zone, and can be operably attached to either or both of the backsheet 204 and bodyside liner 202 of the absorbent article 200. The laterally inboard bonding zone region of each ear 266 can be overlapped and bonded with its corresponding, lateral end edge of the waist region 212 of the absorbent article 200. The ears 266 can extend laterally to form a pair of opposed waist-flap sections of the absorbent article 200 and can be bonded with suitable bonding means, such as adhesive bonding, thermal bonding, ultrasonic bonding, and the like.

The ears 266 can be constructed from a non-elastomeric material, such as polymer films, woven materials, nonwoven materials, and combinations thereof. In an embodiment, the ears 266 can be constructed from a substantially elastomeric material, such as a stretch-bonded laminate (SBL) material, a neck-bonded laminate (NBL) material, an elastomeric film, an elastomeric foam material, or the like, which is elastomerically stretchable at least along the lateral direction 226.

For example, suitable meltblown elastomeric fibrous webs for forming ears 266 are described in U.S. Pat. No. 4,663,220 to Wisneski et al., the entire disclosure of which is incorporated herein by reference. Examples of composite fabrics comprising at least one layer of nonwoven textile fabric secured to a fibrous elastic layer are described in EP 0217032 A2 to Taylor et al., the entire disclosure of which is incorporated herein by reference. Examples of NBL materials are described in U.S. Pat. No. 5,226,992 to Mormon, the entire disclosure of which is incorporated herein by reference.

As described herein, various suitable methods can be employed to bond the ears 266 to the selected portions of the absorbent article 200. Some examples of suitable constructions for bonding a pair of elastically stretchable ears to the lateral side portions of the absorbent article 200 to extend laterally outward beyond the side edges of the backsheet 204 and bodyside liner 202 of the absorbent article 200 can be found in U.S. Pat. No. 4,938,753 to VanGompel, et al., the entire disclosure of which is hereby incorporated by reference in a manner that is consistent herewith.

Each of the ears 266 can extend laterally at one of the opposed lateral ends of at least one of the front or back waist regions, 210 or 212, of the absorbent article 200. In the non-limiting illustration of FIGS. 11 and 12, ears 266 are illustrated extending laterally at the opposed lateral ends of the back waist region 212 of the absorbent article 200. Additionally, a second pair of ears 266 may be included to extend laterally at the opposed lateral ends of the front waist region 210 of the absorbent article 200. The ears 266 can have a tapered, curved or otherwise contoured shape in which the longitudinal length of the relatively inboard base region can be larger or smaller than the longitudinal length of its relatively outboard end region. Alternatively, the ears 266 may have a substantially rectangular shape or may have a substantially trapezoidal shape.

In an embodiment, the ears 266 can include one or more materials bonded together to form a composite ear 266 as is known in the art. For example, the composite ear 266 may be composed of a stretch component 270, a nonwoven carrier or hook base 272, and a male fastening component 274, such as, for example, hooks.

As described above, the mechanical fastening system can have a female component 268. The female component 268 can provide an operable target area for generating a releasable and reattachable securement with at least one male component 274 located on the ears 266. In an embodiment, the female component 268 can be located in the front waist region 210 of the backsheet 204 of the absorbent article 200. In an embodiment, the female component 268 can be directly or indirectly bonded to the backsheet 204 of the absorbent article 200.

In an embodiment, the fluid-entangled laminate web 10 of the present invention can be utilized as the female component 268 of the mechanical fastening system. When used as the female component 268 of a mechanical fastening system, the fluid-entangled laminate web 10 of the present invention can be utilized with a wide variety of male components 274, such as hook materials. Exemplary hook materials suitable for use with the fluid-entangled laminate web 10 are those obtained from: Velcro Group Company, of Manchester, N.H., under the trade designations CFM-23-1098; CFM-22-1121; CFM-22-1162; CFM-25-1003; CFM-29-1003; CFM-29-1005; and CFM-85-1470; or Minnesota Mining & Manufacturing Co., of St. Paul, Minn., under the designation CS 200. Suitable hook materials can generally comprise from about 16, 124, or 155 to about 310, 388, 392, or 620 hooks per square centimeter. The hook materials can have a height of from about 0.00254 cm or 0.0381 cm to about 0.0762 cm or 0.19 cm.

As is known in the art, hook materials can include a base layer with a plurality of uni- or bi-directional hook elements extending generally perpendicular therefrom. As used herein, the term “bi-directional” refers to a hook material having individual adjacent hook elements oriented in opposite directions in the machine direction of the hook material. As used herein, the term “uni-directional” refers to a hook material having individual adjacent hook elements oriented in the same direction in the machine direction of the hook material.

Although the term “hook material” is used herein to designate the portion of the mechanical fastening system having engaging (hook) elements, it is not intended to limit the form of the engaging elements to only include “hooks” but shall encompass any form or shape of engaging element, whether unidirectional or bi-directional, as is known in the art to be designed or adapted to engage a complementary female component 268, such as the fluid-entangled laminate web 10 of the present invention.

Within the fluid-entangled laminate web 10, the fiber material within the land areas 19 can be at least partially entangled together, as described herein, while remaining free of permanent bonds or fusion points, and the fiber material within the projections 12 can be substantially or completely free of bonding or fusing and can retain their fibrous structure, as described herein. Once the fluid-entangled laminate web 10 of the current invention is formed, by any of the methods described herein or otherwise deemed suitable, it can be bonded to the backsheet 204 of a personal care absorbent article 200, such as, for example, a disposable diaper, a non-limiting illustration of which is shown in FIG. 11. The fluid-entangled laminate web 10 can be attached to the backsheet 204 of the absorbent article 200 such that at least one of the projections 12 is exposed. The fluid-entangled laminate web 10 can be bonded to the backsheet 204 by any known manner including, but not limited to, adhesives, thermal bonding, ultrasonic bonding, or a combination thereof. In the event that at least one adhesive is selected, a wide variety of adhesives can be employed, including, but not limited to, solvent-based, water-based, hot-melt and pressure sensitive adhesives. Powdered adhesives can also be applied to the fluid-entangled laminate web 10 and then heated to activate the powder adhesive and perfect bonding.

The tensile strength of a female component 268, defined as the peak load achieved during the test, can be measured in the Machine Direction (MD) according to the Method to Determine Tensile Strength described herein (“MD peak load”). In an embodiment, the fluid-entangled laminate web 10, when utilized as a female component 268 of a mechanical fastening system, can have a MD peak load of greater than about 3000 gf per inch. In an embodiment, the fluid-entangled laminate web 10, when utilized as a female component 268 of a mechanical fastening system, can have a MD peak load of greater than about 3000, 3200, 3400, 3600, 3800, 4000, 4200, 4400, 4600, 4800, or 5000 gf per inch. In an embodiment, the fluid-entangled laminate web 10, when utilized as a female component 268 of a mechanical fastening system, can have an MD peak load of from about 3000, 3200, 3400, 3600, 3800 or 4000 gf per inch to about 4200, 4400, 4600, 4800, 5000, or 5200 gf per inch.

As described herein, the land area 19 of a fluid-entangled laminate web 10 can have a percentage of open area in which light can pass through the land areas 19 unhindered by the material forming the land areas 19, such as, for example, fibrous material. As described herein, the land area 19 of a fluid-entangled laminate web 10 can have greater than about 1, 2, 3, 4, 5, 6, 7, 8, 9, 10, 11, 12, 13, 14, 15, 16, 17, 18, 19, or 20% open area in a chosen area of the fluid-entangled laminate web 10 as measured according to the Method to Determine Percent Open Area. As described herein, as the percentage of open area in the land area 19 of a fluid-entangled laminate web 10 increases, the MD peak load can also increase. Without being bound by theory, it is believed that the fluid entanglement process forming the fluid-entangled laminate web 10 can result in an accumulation of fibrous material at the base of the projections 12 and this resultant accumulation can result in an increase in the MD peak load, as measured according to the Method to Determine Tensile Strength, of the fluid-entangled laminate web 10. Several attributes can be achieved by the increase in the MD peak load as the percentage of open area increases which can include, but are not limited to, a softer look, a softer feel, and an open structure without a loss of Machine Direction tensile strength.

In an embodiment, the fluid-entangled laminate web 10 can have an MD peak load of greater than about 3000 gf per inch and a land area 19 of the fluid-entangled laminate web 10 can have a percentage of open area of greater than about 4% open area in a chosen area of the fluid-entangled laminate web 10. In an embodiment, the fluid-entangled laminate web 10 can have an MD peak load of greater than about 3400 gf per inch and a land area 19 of the fluid-entangled laminate web 10 can have a percentage of open area of greater than about 8% open area in a chosen area of the fluid-entangled laminate web 10. In an embodiment, the fluid-entangled laminate web 10 can have an MD peak load of greater than about 4000 gf per inch and a land area 19 of the fluid-entangled laminate web 10 can have a percentage of open area of greater than about 18% open area in a chosen area of the fluid-entangled laminate web 10. In an embodiment, the fluid-entangled laminate web 10 can have an MD peak load of greater than about 5000 gf per inch and a land area 19 of the fluid-entangled laminate web 10 can have a percentage of open area of greater than about 20% open area in a chosen area of the fluid-entangled laminate web 10.

In an embodiment, the fluid-entangled laminate web 10 can have an MD peak load of greater than about 3000 gf per inch (as determined according to the Method to Determine Tensile Strength) and a basis weight of less than about 58 gsm. In an embodiment, the fluid-entangled laminate web 10 can have an MD peak load from about 3000, 3200, 3400, 3600, 3800, or 4000 gf per inch to about 4200, 4400, 4600, 4800, 5000, or 5200 gf per inch and a basis weight from about 40, 42, 44, 46, or 48 gsm to about 50, 52, 54, 56 or 58 gsm. Without being bound by theory, it is believed that the fluid-entanglement process forming the fluid-entangled laminate web 10 can result in the need for less material to form the fluid-entangled laminate web 10 without sacrificing the MD tensile strength of the fluid-entangled laminate web 10.

The fluid-entangled laminate webs 10 are, as described herein, manufactured via fluid-entanglement processes while the pattern-unbonded nonwoven undergoes a thermal bonding process which is different from the fluid-entangling process of the current document. Without being bound by theory, it is believed that the thermal bonding process of the pattern-unbonded nonwoven, which bonds the fibers more firmly in place when compared to the fluid-entanglement processes described herein, can result in a decrease in the stretch capability in the machine direction of the pattern unbonded nonwoven web. In an embodiment, the fluid-entangled laminate webs 10 can have a peak stretch in the machine direction greater than about 20%. In an embodiment, the fluid entangled laminate webs 10 can have a peak stretch in the machine direction greater than about 20, 25, 30, 35, 40, 45, 50, 55, 60, 65, 70, 75, 80, 85, or 90%. In an embodiment, the fluid-entangled laminate webs 10 can have a peak stretch in the machine direction from about 20, 25, 30, 35, 40 or 45% to about 50, 55, 60, 65, 70, 75, 80, 85, 90 or 95%.

The peel strength of a female component 268 can be determined to gauge the strength of the female component 268 of a mechanical fastening system and can be determined according to the Method to Determine Peel Strength described herein. The peel strength of a female component 268 is a gauge of its functionality. More specifically, peel strength is a term used to describe the amount of force needed to pull apart the male and female components of a mechanical fastening system. One way to measure the peel strength is to pull one component from the other at a 180 degree angle. In an embodiment, the fluid-entangled laminate web 10, when utilized as a female component 268 of a mechanical fastening system, can have a peel strength of greater than about 150 gf. In an embodiment, the fluid-entangled laminate web 10, when utilized as a female component 268 of a mechanical fastening system, can have a peel strength from about 150, 175, 200, 225 or 250 gf to about 275, 300, 325, 350, 375, 400, 425, or 450 gf.

The shear strength is another measure of the strength of a mechanical fastening system and can be determined according to the Dynamic Shear Strength Test described herein. Shear strength is measured by engaging the male and female components of the mechanical fastening system and exerting a force along the plane defined by the connected surfaces in an effort to separate the two components. In an embodiment, the fluid-entangled laminate web 10, when utilized as a female component 268 of a mechanical fastening system, can have a shear strength of greater than about 2000 gf. In an embodiment, the fluid-entangled laminate web 10, when utilized as a female component 268 of a mechanical fastening system, can have a shear strength of from about 2000, 2200, 2400, 2600, 2800, or 3000 gf to about 3200, 3400, 3600, 3800, 4000, 4200, 4400, or 4600 gf.

In an embodiment, the void space of a projection 12 of a fluid-entangled laminate web 10 can be determined according to the Method to Determine Percent Void Space described herein. In an embodiment, the percentage of void space present in a projection 12 of a fluid-entangled laminate web 10 can be greater than about 60%. In an embodiment, the percentage of void space present in a projection 12 of a fluid-entangled laminate web 10 can be greater than about 60, 65, 70 or 75%. In an embodiment, the percentage of void space in a projection 12 of a fluid-entangled laminate web 10 can be from about 60% or 65% to about 70, 75 or 80%.

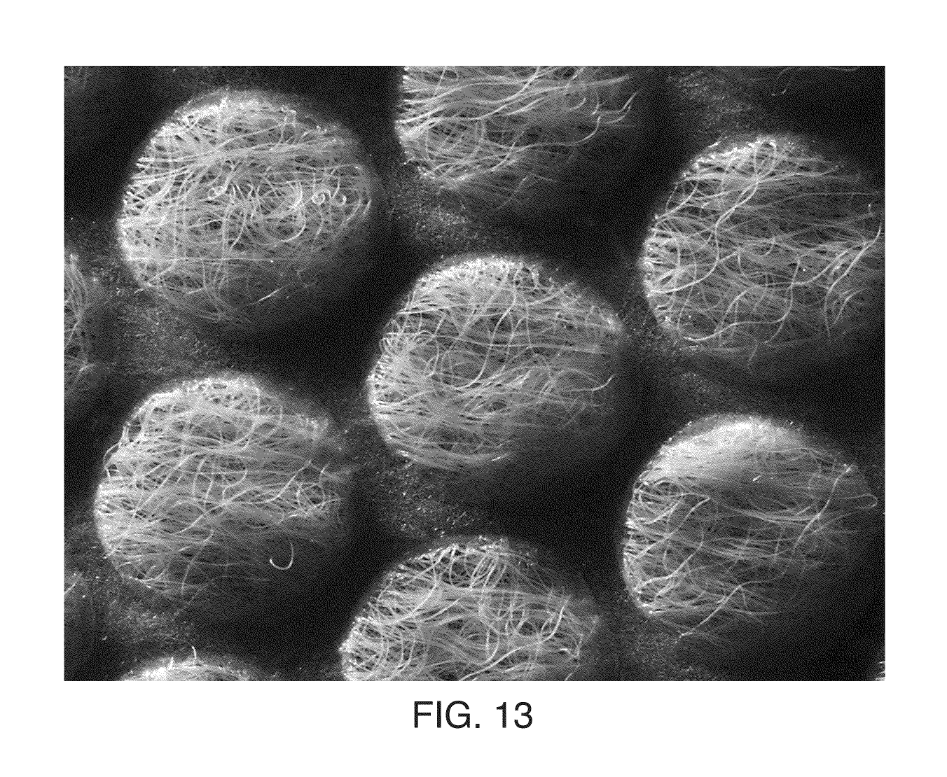

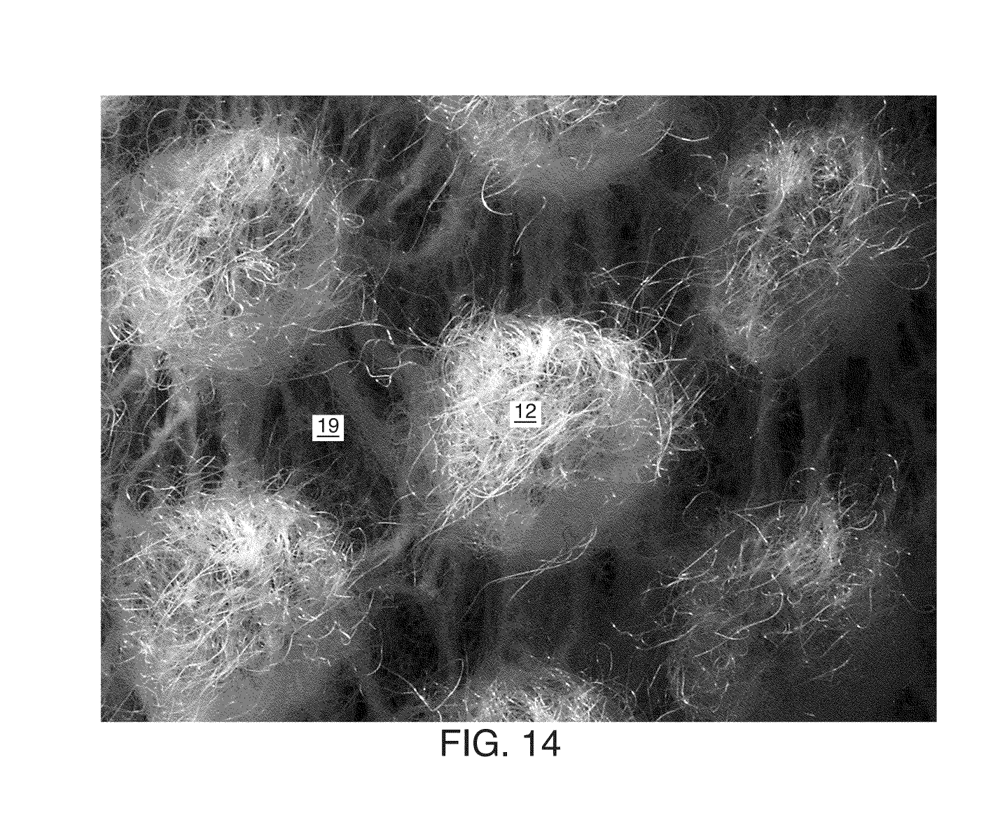

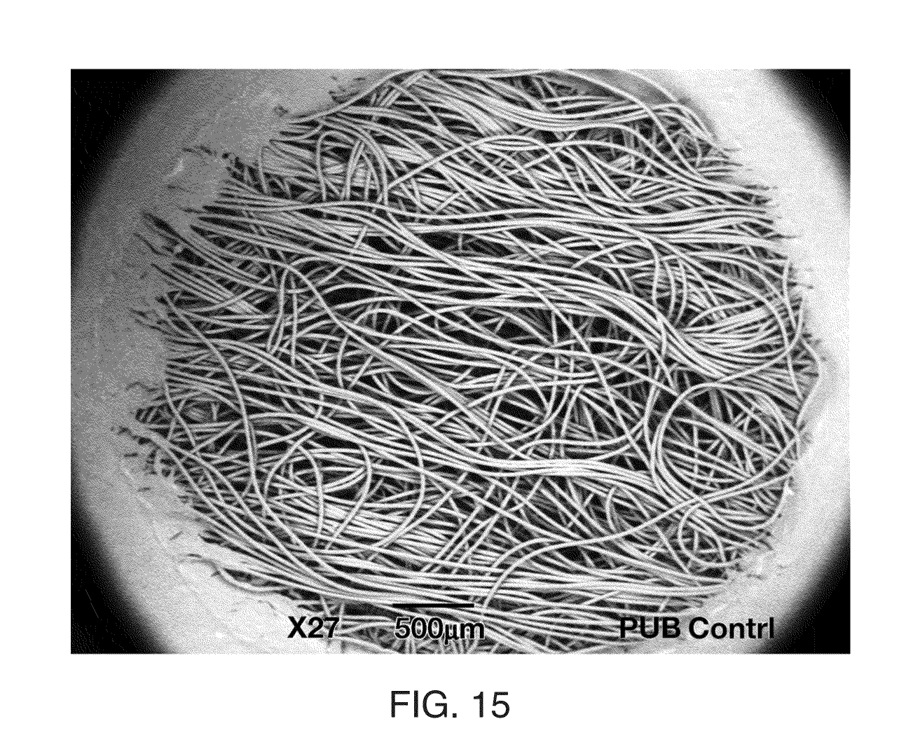

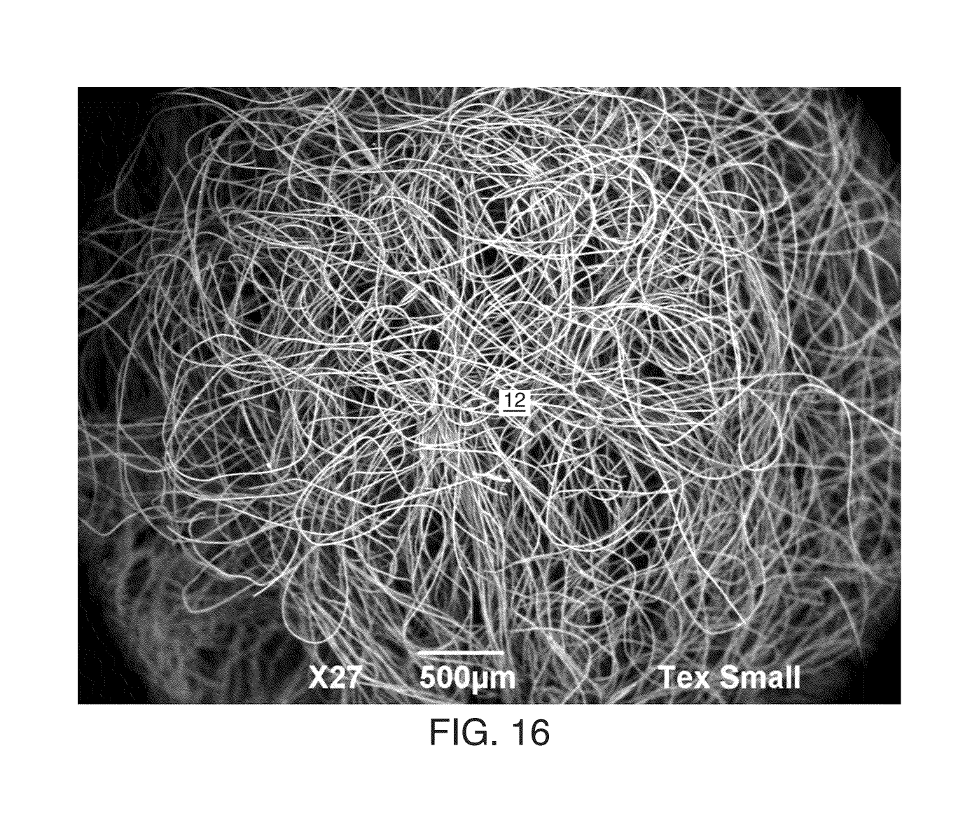

The fluid-entanglement processes described herein can result in a fluid-entangled laminate web 10 which can have lower orientation of the fibers in the fluid-entangled laminate web 10 than other materials currently utilized as the female component 268 of a mechanical fastening system, such as a pattern-unbonded nonwoven material. FIG. 13 is an optical photograph with a horizontal field width of 14.0 mm in top view of a pattern-unbonded nonwoven material and FIG. 14 is an optical photograph with a horizontal field width of 14.0 mm in top view of a fluid-entangled laminate web 10 of the present disclosure. FIGS. 15 and 16 provide SEM images of the top view of the raised area of a pattern unbonded nonwoven web (FIG. 15) and a projection of a fluid-entangled laminate web 10 (FIG. 16). As can be discerned from FIGS. 13-16, the fibers of the pattern unbonded nonwoven web have a higher orientation than the fibers of the fluid-entangled laminate web 10. The orientation of the fluid-entangled laminate web 10 can be described with regard to its field orientation and a fiber segment orientation. The field orientation and the fiber segment orientation can be determined according to the Method to Determine Orientation described herein.

With regard to the field orientation, assuming the machine direction is known during the image acquisition phase, materials which have values greater than 1 are more oriented in the machine direction and materials with orientation values less than 1 are more oriented in the cross direction. Additionally, with regard to the field orientation, materials with orientation values of about 1 are random in their orientation. Additionally, the percent relative standard deviation across rotation values can indicate whether a material has a random orientation or whether the material is more oriented in a specific direction. As described herein, a material which has a random orientation will have a lower percent relative standard deviation across rotation values when compared with a material having greater fiber orientation. With regard to the field orientation of the fluid-entangled laminate web 10, the fluid-entangled laminate web 10 can have a field anisotropy value from about 0.90, 0.91, 0.92, 0.93, 0.94 or 0.95 to about 0.96, 0.97, 0.98, 0.99, 1.00, 1.01, 1.02, 1.03, 1.04 or 1.05. In an embodiment, the fluid-entangled laminate web 10 can have a field anisotropy rotational percent relative standard deviation less than about 20%. In an embodiment, the fluid-entangled laminate web 10 can have a field anisotropy rotational percent relative standard deviation less than about 20, 18, 16, 14, 12, 10, or 8%.

With regard to the fiber segment orientation, the fiber segment orientation is a determination of the orientation of individual fiber segments of the material according to the Method to Determine Orientation described herein. With regard to the orientation of segments of fibers of each of the materials evaluated, a higher value observed for a fiber segment orientation (Feat. Horiz./Vert. Proj.) will provide an indication that the fiber segment orientation is more oriented in the machine direction while a lower value observed for a fiber segment orientation (Feat. Horiz./Vert. Proj.) will provide an indication that the fiber segment orientation is more random or, if low enough, more cross-direction oriented. This concept is further illustrated by reviewing the Feat. Horiz/Vert Proj. rotational percent relative standard deviation. A set of fiber segments which has a random orientation will have a lower rotational percent relative standard deviation than a set of fiber segments which is more oriented, such as in the machine direction. In an embodiment, the fluid-entangled laminate web 10 can have a fiber segment orientation rotational percent relative standard deviation less than about 20%. In an embodiment, the fluid entangled laminate web 10 can have a fiber segment orientation rotational percent relative standard deviation less than about 20, 18, 16, 14, 12, 10, or 8%.

As described herein, the projections 12 can be provided on the fluid-entangled laminate web 10 in any pattern as desired. Without being bound by theory, it is believed that the pattern of projections 12 can influence the peel strength of the fluid-entangled laminate web 10. Without being bound by theory, it is believed that the projections 12 can contribute to the capability of the fluid-entangled laminate web 10 to engage with a male component (such as hooks) of a mechanical fastening system. In an embodiment in which the projections 12 can be spaced too far from each other, without being bound by theory, it is believed that there would be a decrease in the peel strength of the fluid-entangled laminate web 10 when utilized as a female component 268 of a mechanical fastening system. Without being bound by theory, it is believed that if the projections 12 are placed too far apart, fewer fibers in projections 12 would be available for engagement with the male component as there would be an increase in the amount of land area 19 between the projections 12 which are not as readily available for engagement with the male component due to the distance of the land area 19 from the male component when compared with the height of the projections 12. In an embodiment, in which projections 12 are spaced closer to each other, without being bound by theory, it is believed that the peel strength of the fluid-entangled laminate web 10 would increase as more fibers in the projections 12 would be available for engagement by the hooks of the male component.

As described herein, the projections 12 can be provided on the fluid-entangled laminate web 10 in any pattern as desired. Without being bound by theory, it is believed that the pattern of projections 12 can influence the shear strength of the fluid-entangled laminate web 10. Without being bound by theory, it is believed as shear take places, the male component (such as hooks) will have the ability to catch and engage fibers located in the land areas 19 of the fluid-entangled laminate web 10. In an embodiment in which the projections 12 are placed further apart from each other, without being bound by theory, it is believed that the shear strength of the fluid-entangled laminate web 10 would increase as more fibers in the land areas 19 are available for catching and engaging the hooks of the male component. In an embodiment in which the projections 12 are placed close together in a fluid-entangled laminate web 10, without being bound by theory, it is believed that the shear strength of the fluid-entangled laminate web 10 may increase as more fiber will be available for catching and engaging with the fluid-entangled laminate web 10.

### Waist Elastic Members:

In an embodiment, the absorbent article 200 can have waist elastic members, 244 and 246, which can be formed of any suitable elastic material. In such an embodiment, suitable elastic materials can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist elastic members, 244 and 246, may be omitted from the absorbent article 200 without departing from the scope of this disclosure.

### Side Panels:

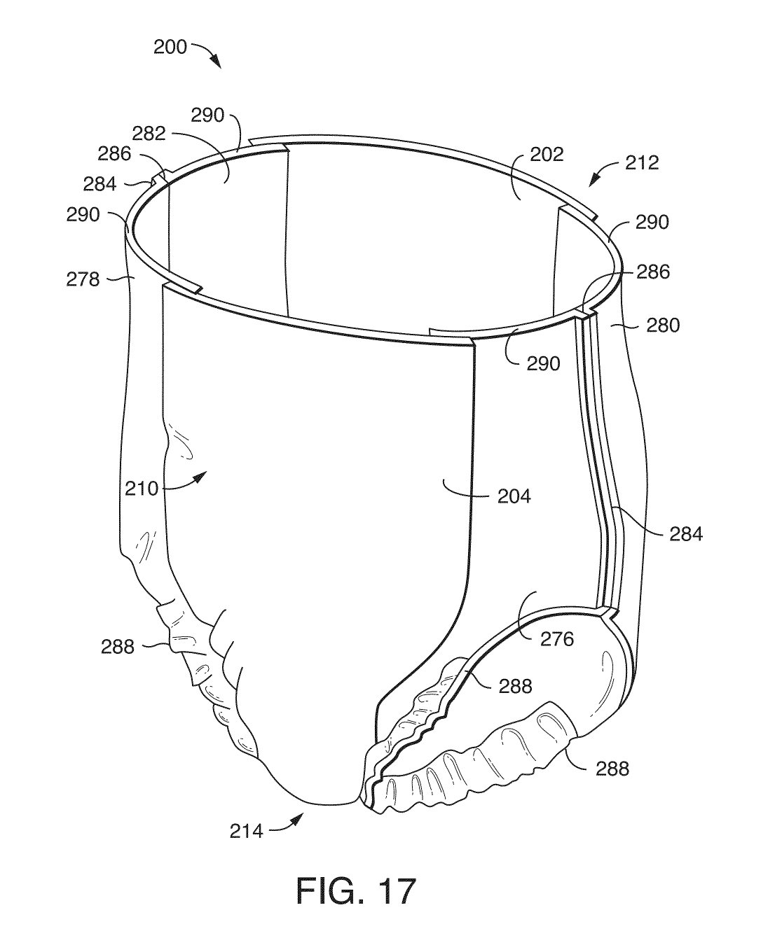

In an embodiment in which the absorbent article 200 can be a training pant, youth pant, diaper pant, or adult absorbent pant, the absorbent article 200 may have front side panels, 276 and 278, and rear side panels, 280 and 282. FIG. 17 provides a non-limiting illustration of an absorbent article 200 that can have side panels, such as front side panels, 276 and 278, and rear side panels, 280 and 282. The front side panels 276 and 278 and the rear side panels 280 and 282 of the absorbent article 200 can be bonded to the absorbent article 200 in the respective front and back waist regions, 210 and 212, and can extend outwardly beyond the longitudinal side edges, 216 and 218, of the absorbent article 200. In an example, the front side panels, 276 and 278, can be bonded to the inner layer 254 of the backsheet 204, such as being bonded thereto by adhesive, by pressure bonding, by thermal bonding or by ultrasonic bonding. These front side panels, 276 and 278, may also be bonded to the outer layer 252 of the backsheet 204, such as by being bonded thereto by adhesive, by pressure bonding, by thermal bonding, or by ultrasonic bonding. The back side panels, 280 and 282, may be secured to the outer and inner layers, 252 and 254 respectively, of the backsheet 204 at the back waist region 212 of the absorbent article 200 in substantially the same manner as the front side panels, 276 and 278. Alternatively, the front side panels, 276 and 278, and the back side panels, 280 and 282, may be formed integrally with the absorbent article 200, such as by being formed integrally with the backsheet 204, the bodyside liner 202 or other layers of the absorbent article 200.

For improved fit and appearance, the front side panels, 276 and 278, and the back side panels, 280 and 282, can suitably have an average length measured parallel to the longitudinal axis of the absorbent article 200 that is about 20 percent or greater, and more suitably about 25 percent or greater, of the overall length of the absorbent article 200, also measured parallel to the longitudinal axis. For example, absorbent articles 200 having an overall length of about 54 centimeters, the front side panels, 276 and 278, and the back side panels, 280 and 282, suitably have an average length of about 10 centimeters or greater, and more suitably have an average length of about 15 centimeters. Each of the front side panels, 276 and 278, and back side panels, 280 and 282, can be constructed of one or more individual, distinct pieces of material. For example, each front side panel, 276 and 278, and back side panel, 280 and 282, can include first and second side panel portions (not shown) joined at a seam (not shown), with at least one of the portions including an elastomeric material. Alternatively, each individual front side panel, 276 and 278, and back side panel, 280 and 282, can be constructed of a single piece of material folded over upon itself along an intermediate fold line (not shown).

The front side panels, 276 and 278, and back side panels, 280 and 282, can each have an outer edge 284 spaced laterally from the engagement seam 286, a leg end edge 288 disposed toward the longitudinal center of the absorbent article 200, and a waist end edge 290 disposed toward a longitudinal end of the absorbent article 200. The leg end edge 288 and waist end edge 290 can extend from the longitudinal side edges, 216 and 218, of the absorbent article 200 to the outer edges 284. The leg end edges 288 of the front side panels, 276 and 278, and back side panels, 280 and 282, can form part of the longitudinal side edges, 216 and 218, of the absorbent article 200. The leg end edges 288 of the illustrated absorbent article 200 can be curved and/or angled relative to the transverse axis to provide a better fit around the wearer's legs. However, it is understood that only one of the leg end edges 288 can be curved or angled, such as the leg end edge 288 of the back waist region 212, or neither of the leg end edges 288 can be curved or angled, without departing from the scope of this disclosure. The waist end edges 290 can be parallel to the transverse axis. The waist end edges 290 of the front side panels, 276 and 278, can form part of the front waist edge 220 of the absorbent article 200, and the waist end edges 290 of the back side panels, 280 and 282, can form part of the back waist edge 222 of the absorbent article 200.

The front side panels, 276 and 278, and back side panels, 280 and 282, can include an elastic material capable of stretching laterally. Suitable elastic materials, as well as one described process for incorporating elastic front side panels, 276 and 278, and back side panels, 280 and 282, into an absorbent article 200 are described in the following U.S. Pat. No. 4,940,464 issued Jul. 10, 1990 to Van Gompel et al., U.S. Pat. No. 5,224,405 issued Jul. 6, 1993 to Pohjola, U.S. Pat. No. 5,104,116 issued Apr. 14, 1992 to Pohjola, and U.S. Pat. No. 5,046,272 issued Sep. 10, 1991 to Vogt et al.; all of which are incorporated herein by reference. As an example, suitable elastic materials include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987, in the name of Taylor et al., and PCT Application WO 01/88245 in the name of Welch et al., all of which are incorporated herein by reference. Other suitable materials are described in U.S. Patent Application Publication No. 12/649,508 to Welch et al. and Ser. No. 12/023,447 to Lake et al., all of which are incorporated herein by reference. Alternatively, the front side panels, 276 and 278, and back side panels, 280 and 282, may include other woven or non-woven materials, such as those described above as being suitable for the backsheet 204 or bodyside liner 202, mechanically pre-strained composites, or stretchable but inelastic materials.

### Method to Determine Percent Open Area

The percentage of open area can be determined by using the image analysis measurement method described herein. In this context, the open area is considered the regions within a material where light transmitted from a light source passes directly through those regions unhindered in the material of interest. Generally, the image analysis method determines a numeric value of percent open area for a material via specific image analysis measurement parameters such as area. The percent open area method is performed using conventional optical image analysis techniques to detect open area regions in both land areas and projections separately and then calculating their percentages in each. To separate land areas and projections for subsequent detection and measurement, incident lighting is used along with image processing steps. An image analysis system, controlled by an algorithm, performs detection, image processing and measurement and also transmits data digitally to a spreadsheet database. The resulting measurement data are used to determine the percent open area of materials possessing land areas and projections.

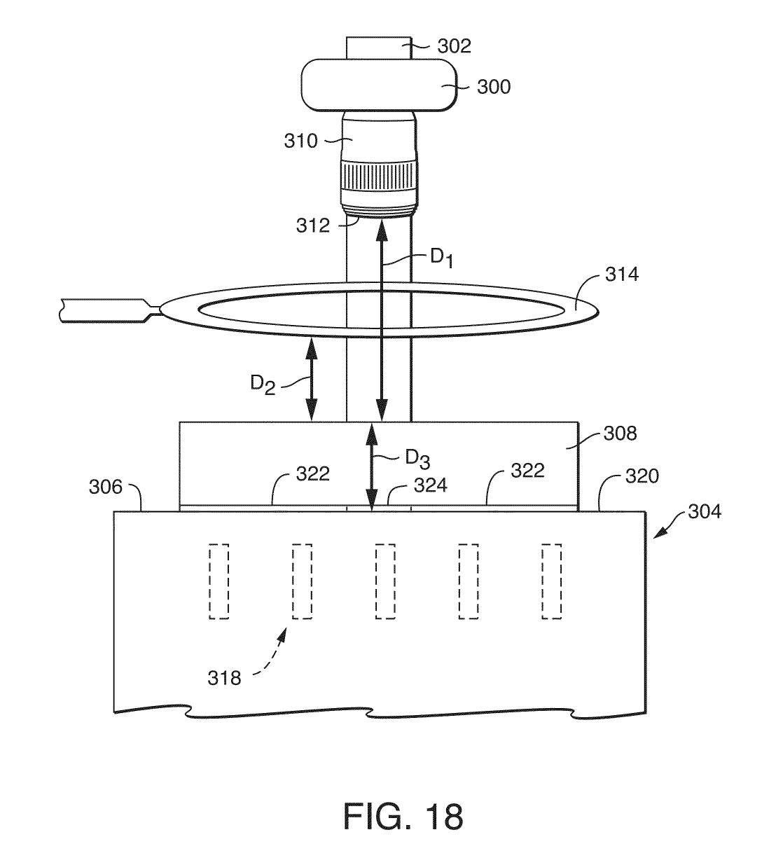

The method for determining the percent open area in both land areas and projections of a given material includes the step of acquiring two separate digital images of the material. An exemplary setup for acquiring the image is representatively illustrated in FIG. 18. Specifically, a CCD video camera 300 (e.g., a Leica DFC 310 FX video camera operated in gray scale mode and available from Leica Microsystems of Heerbrugg, Switzerland) is mounted on a standard support 302 such as a Polaroid MP-4 Land Camera standard support or equivalent available from Polaroid Resource Center in Cambridge, Miss. The standard support 302 is attached to a macro-viewer 304 such as a KREONITE macro-viewer available from Dunning Photo Equipment, Inc., having an office in Bixby, Okla. An auto stage 308 is placed on the upper surface 306 of the macro-viewer 304. The auto stage 308 is used to automatically move the position of a given material for viewing by the camera 300. A suitable auto stage is Model H112, available from Prior Scientific Inc., having an office in Rockland, Mass.

The material possessing land areas and projections is placed on the auto stage 308 under the optical axis of a 60 mm Nikon AF Micro Nikkor lens 310 with an f-stop setting of 4. The Nikon lens 310 is attached to the Leica DFC 310 FX camera 300 using a c-mount adaptor. The distance D1 from the front face 312 of the Nikon lens 310 to the material is 21 cm. The material is laid flat on the auto stage 308 and any wrinkles removed by gentle stretching and/or fastening it to the auto stage 308 surface using transparent adhesive tape at its outer edges. The material is oriented so the machine-direction (MD) runs in the horizontal direction of the resulting image. The material surface is illuminated with incident fluorescent lighting provided by a 16 inch diameter, 40 watt, GE Circline fluorescent lamp 314. The lamp 314 is contained in a fixture that is positioned so it is centered over the material and under the video camera above and is a distance D2 of 3 inches above the material surface. The illumination level of the lamp 314 is controlled with a Variable Auto-transformer, type 3PN1010, available from Staco Energy Products Co., having an office in Dayton, Ohio. Transmitted light is also provided to the material from beneath the auto stage 308 by a bank of five 20 watt fluorescent lights 318 covered with a diffusing plate 320. The diffusing plate 320 is inset into, and forms a portion of, the upper surface 306 of the macro-viewer 304. The diffusing plate 320 is overlaid with a black mask 322 possessing a 3-inch by 3-inch opening 324. The opening 324 is positioned so that it is centered under the optical axis of the Leica camera and lens system. The distance D3 from the opening 324 to the surface of the auto stage 308 is approximately 17 cm. The illumination level of the fluorescent light bank 318 is also controlled with a separate Variable Auto-transformer.

The image analysis software platform used to perform the percent open area measurements is a QWIN Pro (Version 3.5.1) available from Leica Microsystems, having an office in Heerbrugg, Switzerland. The system and images are also calibrated using the QWIN software and a standard ruler with metric markings at least as small as one millimeter. The calibration is performed in the horizontal dimension of the video camera image. Units of millimeters per pixel are used for the calibration.

The method for determining the percent open area of a given material includes the step of performing several area measurements from both incident and transmitted light images. Specifically, an image analysis algorithm is used to acquire and process images as well as perform measurements using Quantimet User Interactive Programming System (QUIPS) language. The image analysis algorithm is reproduced below.

```
NAME = % Open Area - Land vs Projection Regions-1
PURPOSE = Measures % open area on ‘land’ and ‘projection’ regions via ‘sandwich’ lighting
technique
DEFINE VARIABLES & OPEN FILES
Open File ( C:\Data\39291\% Open Area\data.xls, channel #1 )
MFLDIMAGE = 2
TOTCOUNT = 0
TOTFIELDS = 0
SAMPLE ID AND SET UP
Configure ( Image Store 1392 × 1040, Grey Images 81, Binaries 24 )
Enter Results Header
File Results Header ( channel #1 )
File Line ( channel #1 )
Image Setup DC Twain [PAUSE] ( Camera 1, AutoExposure Off, Gain 0.00,
ExposureTime 34.23 msec, Brightness 0, Lamp 38.83 )
Measure frame ( x 31, y 61, Width 1330, Height 978 )
Image frame ( x 0, y 0, Width 1392, Height 1040 )
-- Calvalue = 0.0231 mm/px
CALVALUE = 0.0231
Calibrate ( CALVALUE CALUNITS$ per pixel )
Clear Accepts
For ( SAMPLE = 1 to 1, step 1 )
  Clear Accepts
  File ( “Field No.”, channel #1, field width: 9, left justified )
  File ( “Land Area”, channel #1, field width: 9, left justified )
  File ( “Land Open Area”, channel #1, field width: 13, left justified )
  File ( “%Open Land Area”, channel #1, field width: 15, left justified )
  File ( “Proj. Area”, channel #1, field width: 9, left justified )
  File ( “Proj. Open Area”, channel #1, field width: 13, left justified )
  File ( “% Open Proj. Area”, channel #1, field width: 15, left justified )
  File ( “Total % Open Area”, channel #1, field width: 14, left justified )
  File Line ( channel #1 )
  Stage ( Define Origin )
Stage ( Scan Pattern, 5 × 1 fields, size 82500.000000 × 82500.000000 )
IMAGE ACQUISITION I - Projection isolation
For ( FIELD = 1 to 5, step 1 )
  Display ( Image0 (on), frames (on,on), planes (off,off,off,off,off,off), lut 0, x 0, y 0, z
  1, Reduction off )
  PauseText ( “Ensure incident lighting is correct (WL = 0.88 - 0.94) and acquire
  image.” )
  Image Setup DC Twain [PAUSE] ( Camera 1, AutoExposure Off, Gain 0.00,
  ExposureTime 34.23 msec, Brightness 0, Lamp 38.83 )
  Acquire ( into Image0 )
  DETECT - Projections only
  PauseText ( “Ensure that threshold is set at least to the right of the left gray-level
    histogram peak which corresponds to the ‘land’ region.” )
  Detect [PAUSE] ( whiter than 127, from Image0 into Binary0 delineated )
BINARY IMAGE PROCESSING
Binary Amend (Close from Binary0 to Binary1, cycles 10, operator Disc, edge erode on)
Binary Identify ( FillHoles from Binary1 to Binary1 )
Binary Amend (Open from Binary1 to Binary2, cycles 20, operator Disc, edge erode on)
Binary Amend (Close from Binary2 to Binary3, cycles 8, operator Disc, edge erode on )
PauseText (“Toggle <control> and <b> keys to check bump detection and correct if
  necessary.” )
Binary Edit [PAUSE] ( Draw from Binary3 to Binary3, nib Fill, width 2 )
Binary Logical ( copy Binary3, inverted to Binary4 )
IMAGE ACQUISITION 2 - % Open Area
Display ( Image0 (on), frames (on,on), planes (off,off,off,off,off,off), lut 0, x 0, y 0, z
  1, Reduction off )
PauseText ( “Turn off incident light & ensure transmitted lighting is correct (WL =
  0.97) and acquire image.” )
Image Setup DC Twain [PAUSE] ( Camera 1, AutoExposure Off, Gain 0.00,
  ExposureTime 34.23 msec, Brightness 0, Lamp 38.83 )
  Acquire ( into Image0 )
  DETECT - Open areas only
  Detect ( whiter than 210, from Image0 into Binary10 delineated )
  BINARY IMAGE PROCESSING
  Binary Logical ( C = A AND B : C Binary11, A Binary3, B Binary10 )
  Binary Logical ( C = A AND B : C Binary12, A Binary4, B Binary10 )
  MEASURE AREAS - Land, projections, open area within each
  -- Land Area
  MFLDIMAGE = 4
  Measure field ( plane MFLDIMAGE, into FLDRESULTS(1), statistics into
    FLDSTATS(7,1) ) Selected parameters:  Area
  LANDAREA = FLDRESULTS(1)
  -- Projection Area
  MFLDIMAGE = 3
  Measure field ( plane MFLDIMAGE, into FLDRESULTS(1), statistics into
    FLDSTATS(7,1) ) Selected parameters:  Area
  BUMPAREA = FLDRESULTS(1)
  -- Open Projection area
  MFLDIMAGE = 11
  Measure field ( plane MFLDIMAGE, into FLDRESULTS(1), statistics into
    FLDSTATS(7,1) ) Selected parameters:  Area
  APBUMPAREA = FLDRESULTS(1)
  -- Open land area
  MFLDIMAGE = 12
  Measure field ( plane MFLDIMAGE, into FLDRESULTS(1), statistics into
    FLDSTATS(7,1) ) Selected parameters:  Area
  APLANDAREA = FLDRESULTS(1)
  -- Total % open area
  MFLDIMAGE = 10
  Measure field ( plane MFLDIMAGE, into FLDRESULTS(1), statistics into
    FLDSTATS(7,1) ) Selected parameters:  Area%
  TOTPERCAPAREA = FLDRESULTS(1)
  CALCULATE AND OUTPUT AREAS
  PERCAPLANDAREA = APLANDAREA/LANDAREA*100
  PERCAPBUMPAREA = APBUMPAREA/BUMPAREA*100
  File ( FIELD, channel #1, 0 digits after ‘.’ )
  File ( LANDAREA, channel #1, 2 digits after ‘.’ )
  File ( APLANDAREA, channel #1, 2 digits after ‘.’ )
  File ( PERCAPLANDAREA, channel #1, 1 digit after ‘.’ )
  File ( BUMPAREA, channel #1, 2 digits after ‘.’ )
  File ( APBUMPAREA, channel #1, 4 digits after ‘.’ )
  File ( PERCAPBUMPAREA, channel #1, 5 digits after ‘.’ )
  File ( TOTPERCAPAREA, channel #1, 2 digits after ‘.’ )
  File Line ( channel #1 )
  Stage ( Step, Wait until stopped + 1100 msecs )
  Next ( FIELD )
  PauseText ( “If no more samples, enter ‘0.’” )
  Input ( FINISH )
  If ( FINISH=0 )
  Goto OUTPUT
  Endif
  PauseText ( “Place the next replicate specimen on the auto-stage, turn on incident light
  and turn-off and/or block sub-stage lighting.” )
  Image Setup DC Twain [PAUSE] ( Camera 1, AutoExposure Off, Gain 0.00,
  ExposureTime 34.23 msec, Brightness 0, Lamp 38.83 )
  File Line (channel #1)
Next ( SAMPLE )
OUTPUT:
Close File ( channel #1 )
END
```

The QUIPS algorithm is executed using the QWIN Pro software platform. The analyst is initially prompted to enter the material set information which is sent to the EXCEL file.

The analyst is next prompted by a live image set up window on the computer monitor screen to place a material onto the auto-stage 308. The material should be laid flat and gentle force applied at its edges to remove any macro-wrinkles that may be present. It should also be aligned so that the machine direction runs horizontally in the image. At this time, the Circline fluorescent lamp 314 can be on to assist in positioning the material. Next, the analyst is prompted to adjust the incident Circline fluorescent lamp 314 via the Variable Auto-transformer to a white level reading of approximately 0.9. The sub-stage transmitted light bank 318 should either be turned off at this time or masked using a piece of light-blocking, black construction paper placed over the 3 inch by 3 inch opening 324.

The analyst is now prompted to ensure that the detection threshold is set to the proper level for detection of the projections using the Detection window which is displayed on the computer monitor screen. Typically, the threshold is set using the white mode at a point approximately near the middle of the 8-bit gray-level range (e.g. 127). If necessary, the threshold level can be adjusted up or down so that the resulting detected binary will optimally encompass the projections shown in the acquired image with respect to their boundaries with the surrounding land region.

After the algorithm automatically performs several binary image processing steps on the detected binary of the projections, the analyst will be given an opportunity to re-check projection detection and correct any inaccuracies. The analyst can toggle both the ‘control’ and ‘b’ keys simultaneously to re-check projection detection against the underlying acquired gray-scale image. If necessary, the analyst can select from a set of binary editing tools (e.g., draw, reject, etc.) to make any minor adjustments. If care is taken to ensure proper illumination and detection in the previously described steps, little or no correction at this point should be necessary.

Next, the analyst is prompted to turn off the incident Circline fluorescent lamp 314 and either turn on the sub-stage transmitted light bank or remove the light blocking mask. The sub-stage transmitted light bank is adjusted by the Variable Auto-transformer to a white level reading of approximately 0.97. At this point, the image focus can be optimized for the land areas of the material.

The algorithm, after performing additional operations on the resulting separate binary images for projections, land areas and open area, will then automatically perform measurements and output the data into a designated EXCEL spreadsheet file. The following measurement parameter data will be located in the EXCEL file after measurements and data transfer has occurred:
Land Area
Land Open Area
Land % Open Area
Projection Area
Projection Open Area
Projection % Open Area
Total % Open Area

Following the transfer of data, the algorithm will direct the auto-stage 308 to move to the next field-of-view and the process of turning on the incident, Circline fluorescent lamp 314 and blocking the transmitted sub-stage lighting bank 318 will begin again. This process will repeat four times so that there will be five sets of data from five separate field-of-view images per single material replicate.

Multiple sampling replicates from a single material can be performed during a single execution of the QUIPS algorithm (Note: The Sample For—Next line in the algorithm needs to be adjusted to reflect the number of material replicate analyses to be performed per material). The final material mean spread value is usually based on an N=5 analysis from five, separate, material subsample replicates. A comparison between different materials can be performed using a Student's T analysis at the 90% confidence level.

### Method for Determining Height of Projections

The height of the projections can be determined by using the image analysis measurement method described herein. The image analysis method determines a dimensional numeric height value for projections using specific image analysis measurements of both land areas and projections with underlying land regions in a sample and then calculating the projection height alone by difference between the two. The projection height method is performed using conventional optical image analysis techniques to detect cross-sectional regions of both land areas and projection structures and then measure a mean linear height value for each when viewed using a camera with incident lighting. The resulting measurement data are used to compare the projection height characteristics of different types of materials.

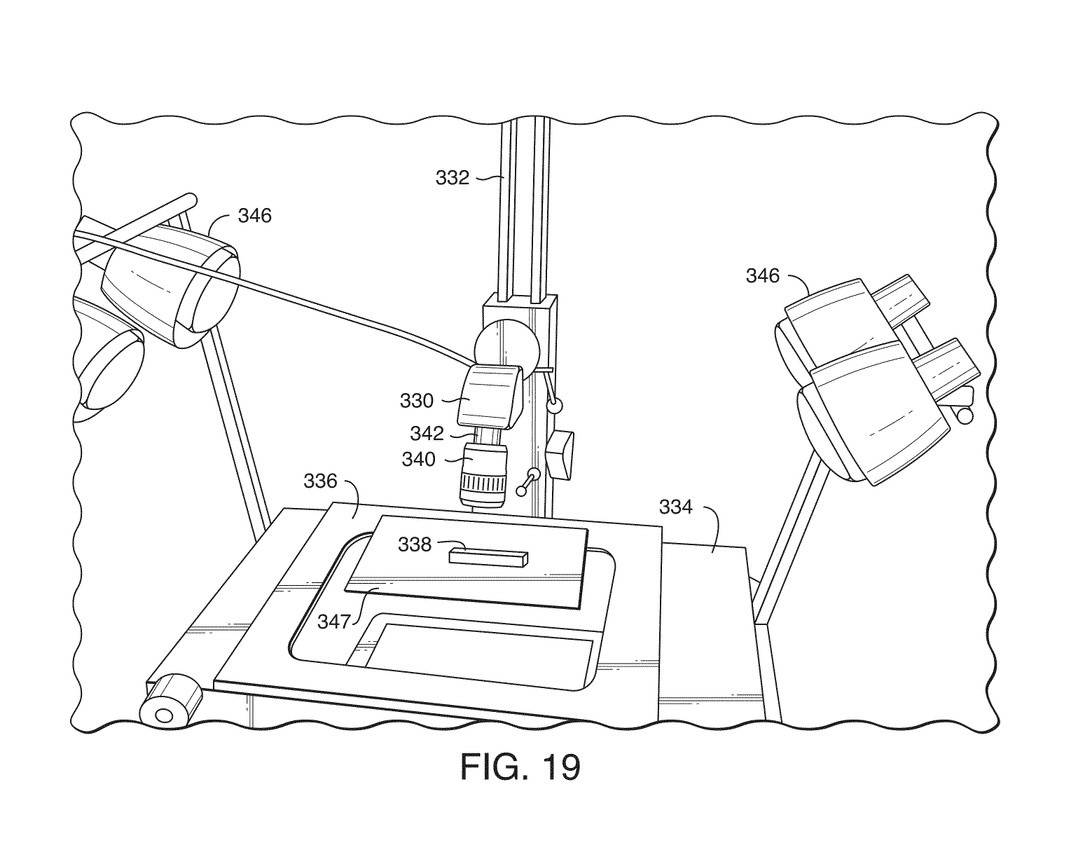

Prior to performing image analysis measurements, the sample of interest must be prepared in such a way to allow visualization of a representative cross-section that passes through the center of a projection. Cross-sectioning can be performed by anchoring a representative piece of the sample on at least one of its cross-machine running straight edges on a flat, smooth surface with a strip of tape such as ¾ inch SCOTCH® Magic™ tape produced by 3M. Cross-sectioning is then performed by using a new, previously unused single edge carbon steel blue blade (PAL) and carefully cutting in a direction away from and orthogonal to the anchored edge and through the centers of at least one projection and preferably more if projections are arranged in rows running in the machine direction. Any remaining rows of projections located behind the cross-sectioned face of projections should be cut away and removed prior to mounting so that only cross-sectioned projections of interest are present. Such blades for cross-sectioning can be acquired from Electron Microscopy Sciences of Hatfield, Pa. (Cat. #71974). Cross-sectioning is performed in the machine-direction of the sample, and a fresh, previously unused blade should be used for each new cross-sectional cut. The cross-sectioned face can now be mounted so that the projections are directed upward away from the base mount using an adherent such as two-side tape so that it can be viewed using a video camera possessing an optical lens. The mount itself and any background behind the sample that will be viewed by the camera must be darkened using non-reflective black tape and black construction paper 347 (shown in FIG. 19), respectively. For a typical sample, enough cross-sections should be cut and mounted separately from which a total of six projection height values can be determined.

An exemplary setup for acquiring the images is representatively illustrated in FIG. 19. Specifically, a CCD video camera 330 (e.g., a Leica DFC 310 FX video camera operated in gray scale mode is available from Leica Microsystems of Heerbrugg, Switzerland) is mounted on a standard support 332 such as a Polaroid MP-4 Land Camera standard support available from Polaroid Resource Center in Cambridge, Miss. or equivalent. The standard support 332 is attached to a macro-viewer 334 such as a KREONITE macro-viewer available from Dunning Photo Equipment, Inc., having an office in Bixby, Okla. An auto stage 336 is placed on the upper surface of the macro-viewer 334. The auto stage 336 is used to move the position of a given sample for viewing by the camera 330. A suitable auto stage 336 is a Model H112, available from Prior Scientific Inc., having an office in Rockland, Mass.

The darkened sample mount 338, exposing the cross-sectioned sample face possessing land areas and projections, is placed on the auto stage 336 under the optical axis of a 50 mm Nikon lens 340 with an f-stop setting of 2.8. The Nikon lens 340 is attached to the Leica DFC 310 FX camera 330 using a 30 mm extension tube 342 and a c-mount adaptor. The sample mount 338 is oriented so the sample cross-section faces flush toward the camera 330 and runs in the horizontal direction of the resulting image with the projections directed upward away from the base mount. The cross-sectional face is illuminated with incident, incandescent lighting 346 provided by two, 150 watt, GE Reflector Flood lamps. The two flood lamps are positioned so that they provide more illumination to the cross-sectional face than to the sample mount 338 beneath it in the image. When viewed from overhead directly above the camera 330 and underlying sample cross-section mount 338, the flood lamps 346 will be positioned at approximately 30 degrees and 150 degrees with respect to the horizontal plane running through the camera 330. From this view the camera support will be at the 90 degree position. The illumination level of the lamps is controlled with a Variable Auto-transformer, type 3PN1010, available from Staco Energy Products Co., having an office in Dayton, Ohio.

The image analysis software platform used to perform measurements is a QWIN Pro (Version 3.5.1) available from Leica Microsystems, having an office in Heerbrugg, Switzerland. The system and images are also calibrated using the QWIN software and a standard ruler with metric markings at least as small as one millimeter. The calibration is performed in the horizontal dimension of the video camera image. Units of millimeters per pixel are used for the calibration.

Thus, the method for determining projection heights of a given sample includes the step of performing several, dimensional measurements. Specifically, an image analysis algorithm is used to acquire and process images as well as perform measurements using Quantimet User Interactive Programming System (QUIPS) language. The image analysis algorithm is reproduced below.

```
NAME = Height - Projection vs Land Regions - 1
PURPOSE = Measures height of projection and land regions
DEFINE VARIABLES & OPEN FILES
-- The following line is set to designate where measurement data will be stored.
Open File (C:\Data\39291\Height\data.xls, channel #1)
FIELDS = 6
SAMPLE ID AND SET UP
Enter Results Header
File Results Header ( channel #1 )
File Line ( channel #1 )
Measure frame ( x 31, y 61, Width 1330, Height 978 )
Image frame ( x 0, y 0, Width 1392, Height 1040 )
-- Calvalue = 0.0083 mm/pixel
CALVALUE = 0.0083
Calibrate ( CALVALUE CALUNITS$ per pixel )
For ( REPLICATE = 1 to FIELDS, step 1 )
  Clear Feature Histogram #1
  Clear Feature Histogram #2
  Clear Accepts
  IMAGE ACQUISITION AND DETECTION
  PauseText ( “Position sample, focus image and set white level to 0.95.” )
  Image Setup DC Twain [PAUSE] ( Camera 1, AutoExposure Off, Gain 0.00,
  ExposureTime 200.00 msec, Brightness 0, Lamp 49.99 )
  Acquire ( into Image0 )
  ACQOUTPUT = 0
  -- The following line can be optionally set-up for saving image files to a specific
  location.
  ACQFILE$ = “C:\Images\39291 - for Height\Text.
  2H_“+STR$(REPLICATE)+“s.jpg”
  Write image ( from ACQOUTPUT into file ACQFILE$ )
  Detect ( whiter than 104, from Image0 into Binary0 delineated )
  IMAGE PROCESSING
  Binary Amend (Close from Binary0 to Binary1, cycles 4, operator Disc, edge erode on)
  Binary Amend (Open from Binary1 to Binary2, cycles 4, operator Disc, edge erode on)
  Binary Identify (FillHoles from Binary2 to Binary3)
  Binary Amend (Close from Binary3 to Binary4, cycles 15, operator Disc, edge erode on)
  Binary Amend (Open from Binary4 to Binary5, cycles 20, operator Disc, edge erode on)
  PauseText ( “Fill in projection & land regions that should be included, and reject over
  detected regions.” )
  Binary Edit [PAUSE] ( Draw from Binary5 to Binary6, nib Fill, width 2 )
  PauseText ( “Select ‘Land’ region for measurement.” )
  Binary Edit [PAUSE] ( Accept from Binary6 to Binary7, nib Fill, width 2 )
  PauseText ( “Select ‘Projection’ region for measurement.” )
  Binary Edit [PAUSE] ( Accept from Binary6 to Binary8, nib Fill, width 2 )
  -- Combine land and projection regions with measurement grid.
  Graphics ( Grid, 30 × 0 Lines, Grid Size 1334 × 964, Origin 21 × 21, Thickness 2,
  Orientation 0.000000, to Binary15 Cleared )
  Binary Logical ( C = A AND B : C Binary10, A Binary7, B Binary15 )
  Binary Logical ( C = A AND B : C Binary11, A Binary8, B Binary15 )
  MEASURE HEIGHTS
  -- Land region only
  Measure feature ( plane Binary10, 8 ferets, minimum area: 8, grey image: Image0 )
    Selected parameters: X FCP, Y FCP, Feret90
  Feature Histogram #1 ( Y Param Number, X Param Feret90, from 0.0100 to 5.,
  logarithmic, 20 bins )
  Display Feature Histogram Results ( #1, horizontal, differential, bins + graph (Y axis
  linear), statistics ) Data Window ( 1278, 412, 323, 371 )
  -- Projection regions only (includes any underlying land material)
  Measure feature ( plane Binary11, 8 ferets, minimum area: 8, grey image: Image0 )
    Selected parameters: X FCP, Y FCP, Feret90
  Feature Histogram #2 ( Y Param Number, X Param Feret90, from 0.0100 to 10.,
  logarithmic, 20 bins )
  Display Feature Histogram Results ( #2, horizontal, differential, bins + graph (Y axis
  linear), statistics ) Data Window ( 1305, 801, 297, 371 )
  OUTPUT DATA
  File ( “Land Height (mm)”, channel #1 )
  File Line ( channel #1 )
  File Feature Histogram Results ( #1, differential, statistics, bin details, channel #1 )
  File Line ( channel #1 )
  File Line ( channel #1 )
  File ( “Projection + Land Height (mm)”, channel #1 )
  File Line ( channel #1 )
  File Feature Histogram Results ( #2, differential, statistics, bin details, channel #1 )
  File Line ( channel #1 )
  File Line ( channel #1 )
  File Line ( channel #1 )
Next ( REPLICATE )
Close File (channel #1)
END
```

The QUIPS algorithm is executed using the QWIN Pro software platform. The analyst is initially prompted to enter sample identification information which is sent to a designated EXCEL file to which the measurement data will also be subsequently sent.

The analyst is then prompted to position the mounted sample cross-section on the auto-stage 336 possessing the darkened background so the cross-sectional face is flush to the camera 330 with projections directed upward and the length running horizontally in the live image displayed on the video monitor screen. The analyst next adjusts the video camera 330 and lens 340 vertical position to optimize the focus of the cross-sectional face. The illumination level is also adjusted by the analyst via the Variable Auto-transformer to a white level reading of approximately 0.95.

Once the analyst completes the above steps and executes the continue command, an image will be acquired, detected and processed automatically by the QUIPS algorithm. The analyst will then be prompted to fill in the detected binary image, using the computer mouse, of any projection and/or land areas shown in the cross-sectional image that should have been included by the previous detection and image processing steps as well as rejecting any over detected regions that go beyond the boundaries of the cross-sectional structure shown in the underlying gray-scale image. To aid in this editing process, the analyst can toggle the ‘control’ and ‘B’ keys on the keyboard simultaneously to turn the overlying binary image on and off to assess how closely the binary matches with the boundaries of the sample shown in the cross-section. If the initial cross-sectioning sample preparation was performed well, little if any manual editing should be required.

The analyst is now prompted to “Select ‘Land’ region for measurement” using the computer mouse. This selection is performed by carefully drawing a vertical line down through one side of a single land area located between or adjacent to projections and then, with the left mouse button still depressed, moving the cursor beneath the land area to its opposite side and then drawing another vertical line upward. Once this has occurred, the left mouse button can be released and the land area to be measured should be filled in with a green coloring. If the vertical edges of the resulting selected region are skewed in any way, the analyst can reset to the original detected binary by clicking on the ‘Undo’ button located within the Binary Edit window and begin the selection process again until straight vertical edges on both sides of the selected land region are obtained.

Similarly, the analyst will next be prompted to “Select ‘Projection’ region for measurement.” The top portion of a projection region adjacent to the previously selected land area is now selected in the same manner that was previously described for a land area selection.

The algorithm will then automatically perform measurements on both selected regions and output the data, in histogram format, into the designated EXCEL spreadsheet file. In the EXCEL file, the histograms for land and projection regions will be labeled “Land Height (mm)” and “Projection+Land Height (mm),” respectively. A separate set of histograms will be generated for each selection of land and projection region pairs.

The analyst will then again be prompted to position the sample and begin the process of selecting different land and projection regions. At this point, the analyst can either use the auto-stage joystick to move the same cross-section to a new sub-sampling position or an entirely different mounted cross-section obtained from the same sample can be positioned on the auto-stage 306 for measurement. The process for positioning the sample and selecting land and projection regions for measurement will occur six times for each execution of the QUIPS algorithm.

A single projection height value is then determined by calculating the numerical difference between the mean values of the separate land and projection region histograms for each single pair of measurements. The QUIPS algorithm will provide six replicate measurement sets of both land and projection regions for a single sample so that six projection height values will be generated per sample. The final sample mean spread value is usually based on an N=6 analysis from six, separate subsample measurements. A comparison between different samples can be performed using a Student's T analysis at the 90% confidence level.

### Method to Determine Orientation

The orientation of fibers within the projection regions of fibrous materials can be determined by using a scanning electron microscope (SEM) and an image analysis measurement method described herein. In this context, fiber orientation is considered only on the projection surface of the sample of interest. Generally, the image analysis method determines a numeric value of orientation for a material via specific image analysis measurement parameters such as field anisotropy or individual fiber segment orientation measurements after automated image processing steps have occurred. The fiber orientation method is performed using surface high-contrast SEM imaging with subsequent image analysis techniques to detect and measure fibers primarily residing within the surfaces of projections located on the projection layer of a substrate. An image analysis system, controlled by an algorithm, performs detection, image processing and measurement and also transmits data digitally to a spreadsheet database. The resulting measurement data are used to compare the fiber orientation values of structures possessing projection and land regions.

The method for determining the fiber orientation of projections in a given sample includes the step of acquiring six digital surface, high-contrast SEM images of the sample. Prior to imaging, six randomly selected subsample regions are cut from a sample material and mounted on conventional sample stubs that will ultimately be placed into a Jeol model JSM-6490 SEM for imaging. If known, subsample pieces should be mounted on the stubs so that the machine-direction of the material being analyzed is known and marked as such. One way to track directionality is to make small cut outs along directionally designated subsample edges.

Prior to the SEM imaging step, the sample and stub are gold coated using a Denton (Model No. Desk II) sputter coater available from Denton Vacuum, LLC, with an office located in Moorestown, N.J. For example, coating can be performed in five separate application increments with each application being ten seconds in direction. Prior to SEM image acquisition, enough gold should be deposited onto the sample after the regimen is completed so that charging artifacts are not present during imaging.

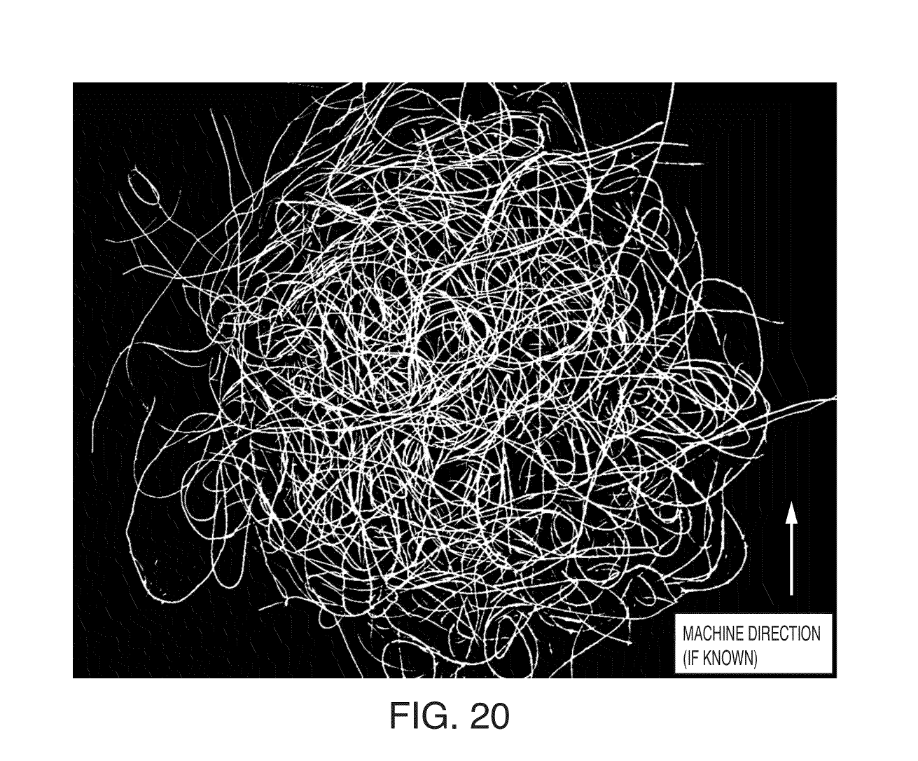

The gold coated sample is now placed into the vacuum imaging chamber of a Jeol model JSM-6490 SEM available from JEOL USA, Inc., having an office in Peabody, Mass. Imaging of the sample surface is performed in backscattered electron mode at 10 kV with a spot size of 55 and a working distance of 15 mm. The sample chamber is set to high vacuum mode. Once these conditions are established, the sample is positioned so that the resulting image will show the center of a projection at the image's center and the machine direction is running vertically. Refer to FIG. 20 which illustrates the approximate type of sampling position required during imaging. The Jeol SEM magnification is typically set to approximately 25× for image acquisition. This setting should be maintained for all samples that will be compared. Six images, one from each of the six randomly selected regions, are acquired per sample. For ease in reading in the images to be analyzed, the image files for a particular sample can be saved using a common prefix name followed by a dash and number designating which of the six replicate images it corresponds to (e.g., XYZ-1). This image file prefix will be used later in the image analysis algorithm to automatically read the six image files to be analyzed. Preferably, all images are saved in tagged image file (TIF) format.

Prior to analysis, images are pre-processed in order to convert the image to a binary black and white version using a commonly available software package such as ImageJ, available via National Institutes of Health (website http://rsb.info.nih.gov/ij/). A gray-scale threshold is set between gray levels 128-255 to convert the high-contrast, eight-bit gray image to a binary where fibers appear as white (i.e., gray level=255) while empty space in between fibers appears as black (i.e., gray level=0). Also, other commonly available image processing packages such as Photoshop or Image Pro can be used for this pre-processing step. The final pre-processing step involves removing unwanted items from the image such as bonded regions that may appear in the pre-processed binary image after thresholding is applied. For this step, an image processing program such as GNU Image Manipulation Program (http://www.gimp.org/) or Photoshop (Adobe Systems Inc.) can be used to blacken bonded regions that are located around the periphery of the central fibrous region from which measurements will be performed.

The image analysis software platform used to perform the fiber orientation measurements is a QWIN Pro (Version 3.5.1) available from Leica Microsystems, having an office in Heerbrugg, Switzerland. The system and images are accurately calibrated using the value provided by the Jeol SEM system in units of microns per pixel. An AGAR Scientific Silicon Test Specimen (No. A877) with 10 micrometer periodicity is used as a calibrating standard. The calibration standard is measured for every sample at the time of analysis at the same working distance, magnification and spot size used to acquire specimen images.

Thus, the method for determining the fiber orientation of a given sample includes the step of performing several orientation measurements on the surface, high-contrast images. Specifically, an image analysis algorithm is used to read and process images as well as perform measurements using Quantimet User Interactive Programming System (QUIPS) language. The image analysis algorithm is reproduced below.

```
NAME = Anisotropy & Orientation - Fibrous Matrices - 1
PURPOSE = Measures field anisotropy and fiber segment orientation
OPEN DATA FILES & SET VARIABLES
Open File ( C:\Data\48125\Orientation Data.xls, channel #1 )
ACQOUTPUT = 0
SET-UP AND CALIBRATION
Configure ( Image Store 1280 × 960, Grey Images 36, Binaries 24 )
-- Pixel calibration value = 4.08 um/px
CALVALUE = 4.08
Calibration ( Local )
Image frame ( x 0, y 0, Width 1280, Height 960 )
Measure frame ( x 155, y 27, Width 963, Height 930 )
Enter Results Header
File Results Header ( channel #1 )
File Line ( channel #1 )
File Line ( channel #1 )
-- Enter image file information
PauseText ( “Enter image file prefix name.” )
Input ( TITLE$ )
Clear Feature Histogram #1
Clear Feature Histogram #2
Clear Field Histogram #1
FIELD/ANALYSIS LOOP
For ( FIELD = 1 to 6, step 1 )
IMAGE ACQUISITION & DETECTION
-- Image File location
ACQFILE$ = “C:\Images\48125 \BE Surface\”+TITLE$+“-”+STR$(FIELD)+“s.tif”
Read image ( from file ACQFILE$ into ACQOUTPUT )
ROTATION OF IMAGE LOOP
For ( ROTATE = 0 to 2, step 1 )
  Clear Feature Histogram #2
  Measure frame ( x 155, y 27, Width 963, Height 930 )
  -- Rotation Variables
  GREYUTILIN = 0
  GREYUTILOUT = 1
  ROTATE.ANGLE = ROTATE*45
  ROTATE.SRCX = 639
  ROTATE.SRCY = 479
  ROTATE.DESTX = 639
  ROTATE.DESTY = 479
  ROTATE.WIDTH = 1280
  ROTATE.HEIGHT = 960
  Grey Rotate ( From ROTATE.SRCX, ROTATE.SRCY in GREYUTILIN to
  ROTATE.DESTX, ROTATE.DESTY in GREYUTILOUT, width ROTATE.WIDTH,
  height ROTATE.HEIGHT, by ROTATE.ANGLE deg )
  Display ( Image1 (on), frames (on,on), planes (off,off,off,off,off,off), lut 0, x 0, y 29, z
  1, Reduction off )
  Detect ( whiter than 200, from Image1 into Binary0 )
  IMAGE PROCESSING
  Binary Amend (Close from Binary0 to Binary1, cycles 1, operator Disc, edge erode on )
  Binary Amend ( White Exh. Skeleton from Binary1 to Binary2, cycles 1, operator Disc,
  edge erode on, alg. ‘L’ Type )
  Binary Identify ( Remove White Triples from Binary2 to Binary3 )
  Binary Amend (Prune from Binary3 to Binary4, cycles 3, operator Disc, edge erode on )
  Display ( Image0 (on), frames (on,on), planes (off,off,off,off,4,off), lut 0, x 0, y 0, z 1,
  Reduction off )
  MEASURE FIELD ANISOTROPY
  MFLDIMAGE = 0
  Measure field ( plane MFLDIMAGE, into FLDRESULTS(1), statistics into
  FLDSTATS(7,1) ) Selected parameters: Anisotropy
  ANISOT = FLDRESULTS(1)
  MEASURE FEATURE ORIENTATION
  Clear Accepts
  Measure feature ( plane Binary4, 64 ferets, minimum area: 20, grey image: Image0 )
  Selected parameters: X FCP, Y FCP, VertProj, HorizProj, Perimeter, UserDef1,
  UserDef2
  Feature Expression ( UserDef1 ( all features ), title Orient =
  PHPROJ(FTR)/PVPROJ(FTR) )
Feature Expression ( UserDef2 ( all features ), title Length = PPERIMETER(FTR)/2 )
Feature Histogram #2 ( Y Param UserDef2, X Param UserDef1, from 1.999999955e
−002 to 200., logarithmic, 20 bins )
Display Feature Histogram Results ( #2, horizontal, differential, bins + graph (Y axis
  linear), statistics ) Data Window ( 1329, 566, 341, 454 )
  -- Output data to spreadsheet
  File ( “Rotation Angle = ”, channel #1 )
  File ( ROTATE.ANGLE, channel #1, 0 digits after ‘.’ )
  File Line ( channel #1 )
  File Feature Histogram Results ( #2, differential, statistics, bin details, channel #1 )
  File Line ( channel #1 )
  File Line ( channel #1 )
  File ( “Anisotropy = ”, channel #1 )
  File ( ANISOT, channel #1, 3 digits after ‘.’ )
  File Line ( channel #1 )
  File Line ( channel #1 )
Next ( ROTATE )
File Line ( channel #1 )
Next ( FIELD )
Close File ( channel #1 )
END
```

The QUIPS algorithm is executed using the QWIN Pro software platform. The analyst is initially prompted to enter the sample set information which is sent to the EXCEL file.

The analyst is then prompted to enter the image file prefix name for those images previously acquired using the Jeol SEM and then pre-processed for a particular sample (e.g., XYZ). Following automated image processing and analysis and transfer of data to a designated EXCEL spreadsheet, the algorithm will automatically read in the next and subsequent images automatically and repeat the processing, analysis and data transfer steps automatically until all six images have been analyzed.

After the algorithm completes analysis of all six images, data will reside in the designated EXCEL spreadsheet file. In the spreadsheet, the following measurement data will be shown from each image at rotation angles of zero, 45 and 90 degrees: Individual fiber segment horizontal/vertical projection ratios shown in a histogram format and field anisotropy values. Using EXCEL, the analyst can now process data such that field anisotropy and horizontal/vertical project data corresponds to both image number and rotation angle. In addition, statistical data such as average, standard deviation and percent relative standard deviation can be calculated. The following data table example (Table 1) shows how data can be organized for further processing:

TABLE 1
Sample Data Table

| | Field Anisotropy | | | | | | Feature Horizontal/Vertical Projection | | | | | |
|---|---|---|---|---|---|---|---|---|---|---|---|---|
| Image | | | | | S. | | | | | | S. | |
| No. | 0° | 45° | 90° | Mean | Dev. | % RSD | 0° | 45° | 90° | Mean | Dev. | % RSD |
| 1 | 0.93 | 1.01 | 1.07 | 1.01 | 0.07 | 7.2 | 1.58 | 2.03 | 1.89 | 1.83 | 0.23 | 12.6 |
| 2 | 1.00 | 0.89 | 1.00 | 0.96 | 0.07 | 6.7 | 1.50 | 1.49 | 1.73 | 1.57 | 0.14 | 8.6 |
| 3 | 0.97 | 1.02 | 1.03 | 1.01 | 0.03 | 3.4 | 1.76 | 1.83 | 1.81 | 1.80 | 0.04 | 2.0 |
| 4 | 0.92 | 1.03 | 1.10 | 1.01 | 0.09 | 8.8 | 1.47 | 2.11 | 2.16 | 1.91 | 0.38 | 20.1 |
| 5 | 0.96 | 1.02 | 1.05 | 1.01 | 0.05 | 4.8 | 1.76 | 2.05 | 1.79 | 1.87 | 0.16 | 8.5 |
| 6 | 0.93 | 0.97 | 1.07 | 0.99 | 0.07 | 7.1 | 1.59 | 1.73 | 1.98 | 1.77 | 0.20 | 11.2 |
| Mean = | 0.95 | 0.99 | 1.05 | | | 6.3 | 1.61 | 1.87 | 1.89 | | | 10.5 |
| S. Dev. = | 0.03 | 0.05 | 0.03 | | | 1.9 | 0.12 | 0.24 | 0.16 | | | 5.9 |
| % RSD = | 3.30 | 5.40 | 3.25 | | | 30.3 | 7.76 | 12.7 | 8.3 | | | 56.6 |

If the machine directions of the samples to be compared are known, data acquired at the zero degree rotation angle for both measurements can be compared directly as a sufficient means to assess any differences between samples. However, if the machine direction of one or more samples to be compared is not known, then percent relative standard deviation values across rotation angles for both measurements can be used as means to compare orientation properties between samples. For example, a sample possessing fairly random fiber orientation will have a low percent relative standard deviation value across rotation angles relative to a sample with a significantly greater fiber orientation.

In order to compare orientation data between samples, a Student's T analysis at the 90% confidence level can be performed using a N=6 value designated by the six subsample replicates performed per sample.

### Method to Determine Percent Void Space

The percentage of void space within the fibrous matrix of the projection-like structures can be determined by using the scanning electron microscope (SEM) and image analysis measurement method described herein. In this context, percent void space is considered only within the region of fibers that make up projection-like structures within the specimen of interest. The method assesses projections both with and without a backing or support layer. Generally, the image analysis method determines a numeric value of percent voids for a material via specific image analysis measurement parameters of a region of interest area and void space area within the overall z-plane region of interest. The projection percent void method is performed using cross-sectional high-contrast SEM imaging with subsequent image analysis techniques to detect both fibers and void space within a selected projection region of interest. An image analysis system, controlled by an algorithm, performs detection, image processing and measurement and also transmits data digitally to a spreadsheet database. The resulting measurement data are used to compare projection percent void values of structures possessing projections and land regions.

The method for determining the percent voids within fibrous projections in a given sample includes the step of acquiring six digital cross-sectional, high-contrast SEM images of the sample. Prior to imaging, samples possessing projections are cross-sectioned through the centers of one or more projections, typically in the machine direction of the material, in order to view the projection in the z-plane of the material. Cross-sectioning is typically performed at room temperature using a new, previously unused stainless steel razor blade such as a GEM #62-0167 available from Electron Microscopy Sciences (Catalog #71972). The sample is then mounted on a conventional cross-sectional sample stub that will ultimately be placed into a Jeol model JSM-6490 SEM for imaging. Typically, six randomly chosen cross-sections will be performed per sample code to be measured.

Prior to the SEM imaging step, the sample and stub are gold coated using a Denton (Model No. Desk II) sputter coater available from Denton Vacuum, LLC, with an office located in Moorestown, N.J. For example, coating can be performed in five separate application increments with each application being ten seconds in duration. Prior to SEM image acquisition, enough gold should be deposited onto the sample after the regimen is completed so that charging artifacts are not present during imaging.

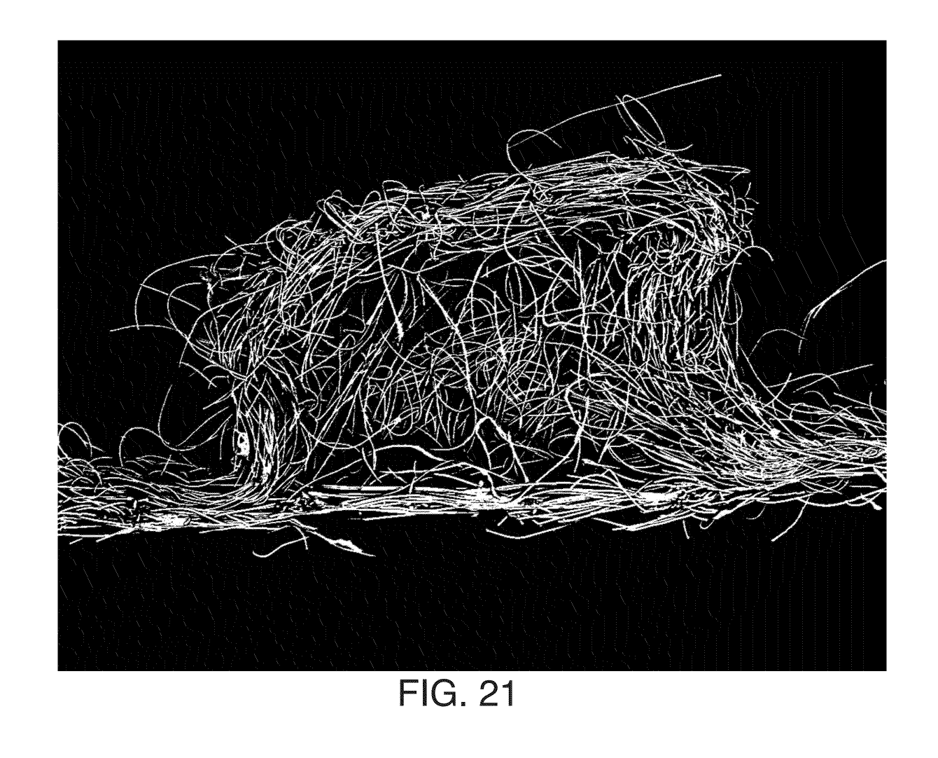

The gold coated sample is now placed into the vacuum imaging chamber of a Jeol model JSM-6490 SEM available from JEOL USA, Inc., having an office in Peabody, Mass. Imaging of the cross-section is performed in backscattered electron mode at 10 kV with a spot size of 55 and a working distance of 15 mm. The sample chamber is set to high vacuum mode. Once these conditions are established, the sample is positioned so that the resulting image will show a single projection located at its approximate center. Refer to FIG. 21, which illustrates the approximate type of sampling position and the image that results. When properly aligned, the machine direction of the sample should run horizontally in the cross-sectional image. The Jeol SEM magnification is typically set to approximately 25× for image acquisition. If possible, this setting should be maintained for all samples that will be compared. Six images, one from each of the six randomly cross-sectioned regions, are acquired per sample. For ease in reading in the images to be analyzed, the image files for a particular sample can be saved using a common prefix name followed by a dash and number designating which of the six replicate images it corresponds to (e.g., XYZ-1). This image file prefix will be used by the image analysis algorithm to automatically read the six image files to be analyzed. Preferably, all images are saved in tagged image file (TIF) format.

Prior to analysis, images are pre-processed in order to convert the image to a binary black and white version using a commonly available software package such as ImageJ, available via National Institutes of Health website http://rsb.info.nih.gov/ij/. A gray-scale threshold is set between gray levels 128-255 to convert the high-contrast, eight-bit gray image to a binary where fibers appear as white (i.e. gray level=255) while empty space in between fibers appears as black (i.e. gray level=0). Also, other commercially available image processing packages such as Photoshop (Adobe Systems Inc.) or Image Pro (Media Cybernetics) can be used for this pre-processing thresholding step. The final pre-processing step involves removing certain unwanted items in the cross-sectional image such as portions of fibers that are entirely detached from the overall projection structure. For this step, an image processing program such as GNU Image Manipulation Program (http://www.gimp.org/) or Photoshop can be used to blacken any unattached fibers.

The image analysis software platform used to perform the projection percent void measurements is a QWIN Pro (Version 3.5.1) available from Leica Microsystems, having an office in Heerbrugg, Switzerland. The system and images are also accurately calibrated using the value provided by the Jeol SEM system in units of microns per pixel. An AGAR Scientific Silicon Test Specimen (No. A877) with 10 micrometer periodicity is used as a calibrating standard. The calibration standard is measured for every sample at the time of analysis at the same working distance, magnification and spot size used to acquire specimen images.

Thus, the method for determining the projection percent voids of a given specimen includes the step of performing area measurements on the cross-sectional, high-contrast image. Specifically, an image analysis algorithm is used to read and process images as well as perform measurements using Quantimet User Interactive Programming System (QUIPS) language. The image analysis algorithm is reproduced below.

```
NAME = Z - Projection Fiber Density - 1
PURPOSE = Measures fiber density (e.g. % voids) of projections
DEFINE VARIABLES & OPEN FILES
-- Spreadsheet file location for data output
Open File ( C:\Data\48125\Z-fiber density.xls, channel #1 )
FIELDS = 6
SAMPLE ID AND SET UP
Enter Results Header
File Results Header ( channel #1 )
File Line ( channel #1 )
Measure frame ( x 31, y 61, Width 1218, Height 898 )
Image frame ( x 0, y 0, Width 1280, Height 960 )
-- Calibration value = 4.7 um/pixel
CALVALUE = 4.7
Calibration ( Local )
-- Enter image prefix name of images to analyze
PauseText ( “Enter image file prefix name.” )
Input ( TITLE$ )
File ( “Rep. No.”, channel #1, field width: 8, left justified )
File ( “% Voids”, channel #1, field width: 7, left justified )
File Line ( channel #1 )
REPLICATE SAMPLING LOOP
For ( REPLICATE = 1 to FIELDS, step 1 )
  Clear Feature Histogram #1
  Clear Feature Histogram #2
  Clear Accepts
  IMAGE ACQUISITION AND DETECTION
  ACQOUTPUT = 0
  -- Image file location pathway
  ACQFILE$ = “C:\Images\48125 \BE X-sections\% Voids\Textor B1-
  4\”+TITLE$+“-”+STR$(REPLICATE)+“s.tif”
Read image ( from file ACQFILE$ into ACQOUTPUT )
  -- Detect void regions
Detect ( blacker than 127, from Image0 into Binary15 )
-- Detect fibers
  Detect ( whiter than 200, from Image0 into Binary0 )
IMAGE PROCESSING
  PauseText ( “Use mouse to select entire projection region of interest in the structure.”)
  Binary Edit [PAUSE] ( Accept from Binary0 to Binary1, nib Fill, width 2 )
  Binary Amend (Close from Binary1 to Binary2, cycles 12, operator Disc, edge erode
  on)
  Binary Amend (Open from Binary2 to Binary3, cycles 10, operator Disc, edge erode on)
  Binary Identify ( FillHoles from Binary3 to Binary4 )
  Binary Amend (Open from Binary4 to Binary5, cycles 25, operator Disc, edge erode on)
  Binary Amend (Close from Binary5 to Binary6, cycles 25, operator Disc, edge erode on)
  Binary Logical ( C = A AND B : C Binary7, A Binary6, B Binary15 )
  MEASURE ANALYSIS REGIONS
  -- Measure area of Analysis Region
  MFLDIMAGE = 6
  Measure field ( plane MFLDIMAGE, into FLDRESULTS(1), statistics into
  FLDSTATS(7,1) ) Selected parameters:  Area
  ANALYSISAREA = FLDRESULTS(1)
  -- Measure area of voids within analysis region
  MFLDIMAGE = 7
  Measure field ( plane MFLDIMAGE, into FLDRESULTS(1), statistics into
  FLDSTATS(7,1) ) Selected parameters:  Area
  VOIDANALYSISAREA = FLDRESULTS(1)
  PERCVOIDS = VOIDANALYSISAREA/ANALYSISAREA*100
  OUTPUT DATA - to spreadsheet
  File ( REPLICATE, channel #1, field width: 8, left justified, 0 digits after ‘.’)
  File ( PERCVOIDS, channel #1, field width: 7, left justified, 1 digit after ‘.’)
  File Line ( channel #1 )
Next ( REPLICATE )
Close File ( channel #1 )
END
```

The QUIPS algorithm is executed using the QWIN Pro software platform. The analyst is initially prompted to enter the sample set information which is sent to the EXCEL file.

The analyst is then prompted to enter the image file prefix name for those images previously acquired using the Jeol SEM and then pre-processed for a particular sample (e.g., XYZ).

The analyst is now prompted to use the computer mouse to select an entire projection region of interest in the structure. Care should be taken to accept the entire structure which may or may not include a supporting layer beneath the projection. Any land regions protruding horizontally outside of the vertical bounds of the projection should not be included in the acceptance selection.

The algorithm will now automatically perform image processing and measurement steps as well as exporting the resulting percent void data to an EXCEL spreadsheet. In the spreadsheet, percent void data will be associated with the number of the image (i.e., 1-6) from which the measurement was performed.

Following the transfer of data, the algorithm will automatically read in the next image and the analyst will again be prompted to manually select the projection region of interest. This process will repeat five times after the first image until all six images have been analyzed. The final sample mean spread value is usually based on an N=6 analysis from six, separate, subsample replicates. A comparison between different samples can be performed using a Student's T analysis at the 90% confidence level.

### Method to Determine Tensile Strength

The tensile strength of the fluid-entangled laminate web 10 in the Machine Direction can be measured according to this test method where indicated as being measured according to the “Method to Determine Tensile Strength.” The tensile strength in the machine direction can be measured using a machine which has a constant rate of extension tensile frame such as an Instron model 5564 tensile testing device running a Testworks software with a ±1 kN load cell. The initial jaw separation distance (“Gauge Length”) was set at 76±1 millimeters and the crosshead speed was set at 305±10 millimeters per minute. The jaw width was 75 millimeters. Samples were cut to 25 mm width by 300 mm length in the machine direction and each tensile strength test result reported was the average of 10 samples per code. Samples were evaluated at room temperature (about 20 degrees Celsius) and about 50% relative humidity. Excess material was allowed to drop out the ends and sides of the apparatus. Machine direction percentage of stretch for the material at peak load was also determined as the percentage of the initial Gauge Length (initial jaw separation).

### Method to Determine Peel Strength

The 180° peel strength test involves attaching a male component (hook material) to a female component (fluid-entangled laminate web) and then peeling the male component from the female component at a 180° angle. The maximum load needed to disengage the two materials is recorded in grams.

To perform the test, a continuous rate of extension tensile tester with a 5000 gram full scale load is required, such as a Sintech System 2 Computer Integrated Testing System available from Sintech, Inc., having offices in Research Triangle Park, N.C. A 75 mm by 102 mm sample of the female component is placed on a flat, adhesive support surface. A 45 mm by 12.5 mm sample of male component, which is adhesively and ultrasonically secured to a substantially inelastic, nonwoven material, is positioned over and applied to the projection web outer surface of the female component sample. To ensure adequate and uniform engagement of the male component to the female component, a 4½ pound automated roller is rolled over the combined male component and female component for one cycle, with one cycle equaling a forward and a backward stroke of the roller. One end of the male component is secured within the upper jaw of the tensile tester, while the end of the female component directed towards the upper jaw is folded downward and secured within the lower jaw of the tensile tester. The placement of the respective materials within the jaws of the tensile tester should be adjusted such that minimal slack exists in the respective materials prior to activation of the tensile tester. The hook elements of the male component are oriented in a direction generally perpendicular to the intended directions of movement of the tensile tester jaws. The tensile tester is activated at a crosshead speed of 500 mm per minute and the peak load in grams to disengage the male component from the female component at an 180° angle is then recorded.

### Dynamic Shear Strength Test

The dynamic shear strength test involves engaging a male component (hook material) to a female component (fluid-entangled laminate web) and then pulling the male component across the female component's surface. The maximum load required to disengage the male component from the female component is measured in grams.

To conduct this test, a continuous rate of extension tensile tester with a 5000 gram full scale load is required, such as a Sintech System 2 Computer Integrated Testing System. A 75 mm by 102 mm sample of the female component is placed on a flat, adhesive support surface. A 45 mm by 12.5 mm sample of a male component, which is adhesively and ultrasonically secured to a substantially inelastic, nonwoven material, is positioned over and applied to the projection web outer surface of the female component sample. To ensure adequate and uniform engagement of the male component to the female component, a 4½ pound automated roller is rolled over the combined male and female components for five cycles, with one cycle equaling a forward and backward stroke of the roller. One end of the nonwoven material supporting the male component is secured within the upper jaw of the tensile tester, and the end of the female component directed toward the lower jaw is secured within the lower jaw of the tensile tester. The placement of the respective materials within the jaws of the tensile tester should be adjusted such that minimal slack exists in the respective materials prior to activation of the tensile tester. The hook elements of the male component are oriented in a direction generally perpendicular to the intended directions of movement of the tensile tester jaws. The tensile tester is activated at a crosshead speed of 250 mm per minute and the peak load in grams to disengage the male component from the female component is then recorded.

### EXAMPLES

### Example 1

To demonstrate the process, apparatus and materials of the present invention, a series of fluid-entangled laminate webs 10 were made, as well as projection webs 16 without support layers 14. The samples were made on a spunlace production line at Textor Technologies PTY LTD in Tullamarine, Australia, in a fashion similar to that shown in FIG. 5 of the drawings with the exception being that only one projection fluid entangling device 140c was employed for forming the projections 12 in the texturizing zone 144. In addition, the projection web 16 was pre-wetted upstream of the process shown in FIG. 5 and prior to the pre-entangling fluid entangling device 140a using conventional equipment. In this case the pre-wetting was achieved through the use of a single injector set at a pressure of 8 bar. The pre-entangling fluid-entangling device 140a was set at 45 bar, the lamination fluid-entangling device 140b was set at 60 bar, while the single projection fluid-entangling device 140c pressure was varied as set forth in Tables 2 and 3 below at pressures of 140, 160 and 180 bar, depending on the particular sample being run.

For the transport belt 110 in FIG. 5, the pre-entangling fluid-entangling device 140a was set at a height of 10 mm above the transport belt 110. For the lamination forming surface 152, the lamination fluid-entangling device 140b was set at a height of 12 mm above the surface 152 as was the projection fluid-entangling device 140c with respect to the projection forming surface 130.

The projection forming surface 130 was a 1.3 m wide steel texturing drum having a diameter of 520 mm, a drum thickness of 3 mm and a hexagonal close packed pattern of 4 mm round forming holes separated by 6 mm on a center-to-center spacing. The porous inner drum shell 138 was a 100 mesh (100 wires per inch in both directions/39 wires per centimeter in both directions) woven stainless steel mesh wire. The separation or gap between the exterior of the shell 138 and the inside of the drum 130 was 1.5 mm.

The process parameters that were varied were the aforementioned entangling fluid pressures (140, 160 and 180 bar) and degree of overfeed (0%, 11%, 25% and 43%) using the aforementioned overfeed ratio of OF=[(V₁/V₃)−1]×100 where V1 is the input speed of the projection web 16 and V3 is the output speed of the resultant laminate 10.

All samples were run at an exit line or take-off speed (V3) of approximately 25 meters per minute (m/min). V1 is reported in the Tables 2 and 3 for the samples therein. V2 was held constant for all samples in Tables 2 and 3 at a speed equal to V3 or 25 meters per minute. The finished samples were sent through a line drier to remove excess water as is usual in the hydroentanglement process. Samples were collected after the drier and then labeled with a code (see Tables 2 and 3) to correspond to the process conditions used.

Relative to the materials made, as indicated below, some were made with a support layer 14 and others were not and when a support layer 14 was used, there were three variations including a spunbond web, a spunlace web and a through-air bonded carded web (TABCW). The spunbond support layer 14 was a 17 gram per square meter (gsm) polypropylene point bonded web made from 1.8 denier polypropylene spunbond fibers which were subsequently point bonded with an overall bond area per unit area of 17.5%. The spunbond web was made by Kimberly-Clark

Australia of Milsons Point, Australia. The spunbond material was supplied and entered into the process in roll form with a roll width of approximately 130 centimeters. The spunlace web was a 52 gsm spunlace material using a uniform mixture of 70 weight percent, 1.5 denier, 40 mm long viscose staple fibers and 30 weight percent, 1.4 denier, 38 mm long polyester (PET) staple fibers made by Textor Technologies PTY LTD of Tullamarine, Australia. The spunlace material was pre-formed and supplied in roll form and had a roll width of approximately 140 centimeters. The TABCW had a basis weight of 40 gsm and comprised a uniform mixture of 40 weight percent, 6 denier, 51 mm long PET staple fibers and 60 weight percent, 3.8 denier, 51 mm long polyethylene sheath/polypropylene core bicomponent staple fibers made by Textor Technologies PTY LTD of Tullamarine, Australia. In the data below (see Table 2) under the heading “support layer” the spunbond web was identified as “SB”, the spunlace web was identified as “SL” and the TABCW was identified as “S”. Where no support layer 14 was used, the term “None” appears. The basis weights used in the examples should not be considered a limitation on the basis weights that can be used as the basis weights for the support layers may be varied depending on the end applications.

In all cases, the projection web 16 was a carded staple fiber web made from 100% 1.2 denier, 38 mm long polyester staple fibers available from the Huvis Corporation of Daejeon, Korea. The carded web was manufactured in-line with the hydroentanglement process by Textor Technologies PTY LTD of Tullamarine, Australia and had a width of approximately 140 centimeters. Basis weights varied as indicated in Tables 2 and 3 and ranged between 28 gsm and 49.5 gsm, though other basis weights and ranges may be used depending upon the end application. The projection web 16 was identified as the “card web” in the data below in Tables 2 and 3.

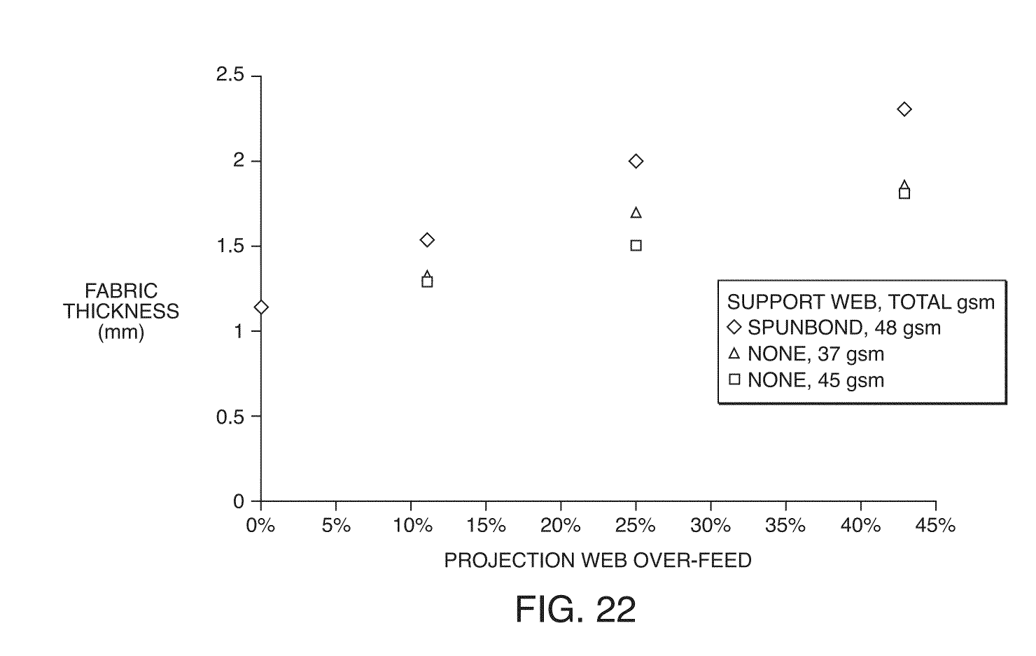

The thickness of the materials set forth in Tables 2 and 3 below, as well as in FIG. 22 of the drawings, were measured using a Mitutoyo model number ID-C1025B thickness gauge with a foot pressure of 345 Pa (0.05 psi). Measurements were taken at room temperature (about 20 degrees Celsius) and reported in millimeters using a round foot with a diameter of 76.2 mm (3 inches). Thicknesses for select samples (average of three samples) with and without support layers are shown in FIG. 22 of the drawings.

The tensile strength of the materials, defined as the peak load achieved during the test, was measured in both the Machine Direction (MD) and the Cross-Machine Direction (CMD) using an Instron model 3343 tensile testing device running an Instron Series 1× software module Rev. 1.16 with a +/−1 kN load cell. The initial jaw separation distance (“Gauge Length”) was set at 75 millimeters and the crosshead speed was set at 300 millimeters per minute. The jaw width was 75 millimeters. Samples were cut to 50 mm width by 300 mm length in the MD and each tensile strength test result reported was the average of two samples per code. Samples were evaluated at room temperature (about 20 degrees Celsius). Excess material was allowed to drape out the ends and sides of the apparatus. CMD strengths and extensions were also measured and generally the CMD strengths were about one half to one fifth of MD strength and CMD extensions at peak load were about two to three times higher than in the MD direction. (The CMD samples were cut with the long dimension being taken in the CMD.) MD strengths were reported in Newtons per 50 mm width of material. (Results are shown in Tables 2 and 3.) MD extensions for the material at peak load were reported as the percentage of the initial Gauge Length (initial jaw separation).

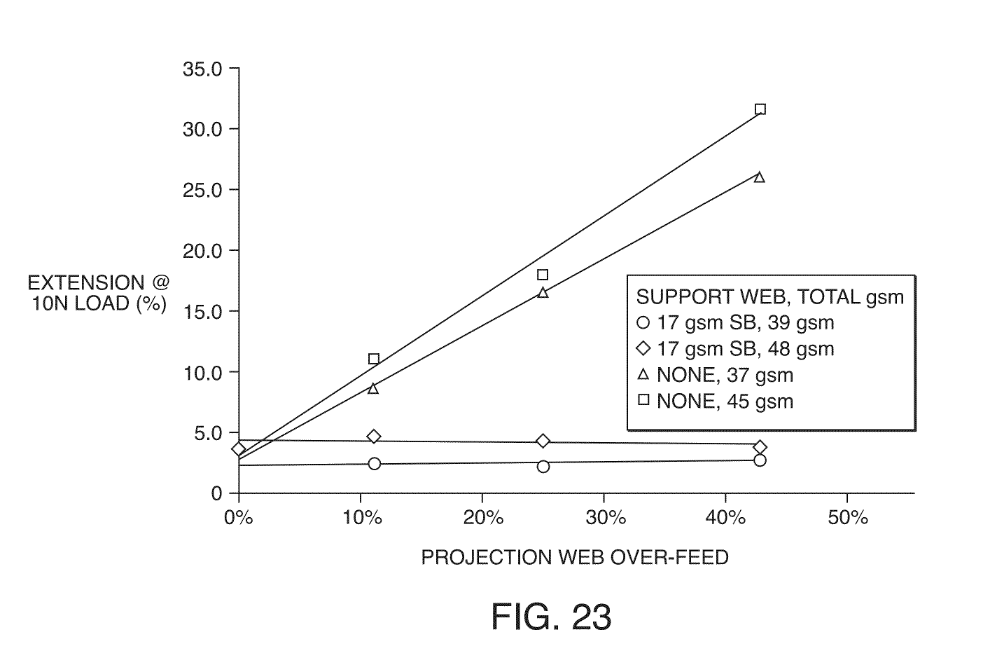

Extension measurements were also made and reported in the MD at a load of 10 Newtons (N). (See Tables 2 and 3 below and FIG. 23). Tables 2 and 3 show data based upon varying the support layer being used, the degree of overfeed being used and variances in the water pressure from the hydroentangling water jets.

As an example of the consequences of varying process parameters, high overfeed requires sufficient jet-pressure to drive the projection web 16 into the texturing drum 130 and so take up the excess material being overfed into the texturing zone 144. If sufficient jet energy is not available to overcome the material's resistance to texturing, then the material will fold and overlap itself and in the worst case may lap a roller prior to the texturing zone 144 requiring the process to be stopped. While the experiments were conducted at a line speed V3 of 25 m/min, this should not be considered a limitation as to the line speed as the equipment with similar materials was run at line speeds ranging from 10 to 70 m/min and speeds outside this range may be used depending on the materials being run.

The following tables summarize the materials, process parameters, and test results. For the samples shown in Table 2, samples were made with and without support layers. Codes 1.1 through 3.6 used the aforementioned spunbond support layer. Codes 4.1 through 5.9 had no support layer. Jet pressures for each of the samples are listed in the Table.

TABLE 2
Experimental parameters and test results, support layer and no support layer, codes 1 to 5.

| | | | | | | | | | Laminate* | Laminate* |
|---|---|---|---|---|---|---|---|---|---|---|
| | | | | | | Laminate* | Laminate* | Laminate* | Extension at | MD |
| | Support | Card web | | Card web Speed | Press. | Weight | Thickness | MD Strength | Peak Load MD | Extension |
| CODE | layer | (gsm) | Overfeed | (V₁) (mm/min) | (bar) | (gsm) | (mm) | (N/50 mm) | (%) | @10 N (%) |
| 1.1 | SB | 28 | 43% | 35.8 | 180 | 51 | 2.22 | 75.6 | 85.0 | 5.0 |
| 1.2 | SB | 28 | 43% | 35.8 | 160 | 52.2 | 2.33 | 65.8 | 82.1 | 3.5 |
| 1.3 | SB | 28 | 43% | 35.8 | 140 | 51.1 | 2.34 | 61.3 | 86.1 | 3.4 |
| 1.4 | SB | 28 | 11% | 27.8 | 140 | 46.3 | 1.47 | 95.5 | 53.0 | 4.9 |
| 1.5 | SB | 28 | 11% | 27.8 | 160 | 45.5 | 1.52 | 91.9 | 46.7 | 4.7 |
| 1.6 | SB | 28 | 11% | 27.8 | 180 | 46.7 | 1.61 | 109.1 | 49.8 | 5.0 |
| 1.7 | SB | 28 | 25% | 31.3 | 180 | 50.5 | 2.02 | 94.4 | 63.7 | 3.7 |
| 1.8 | SB | 28 | 25% | 31.3 | 160 | 50.7 | 1.97 | 82.1 | 62.2 | 5.6 |
| 1.9 | SB | 28 | 25% | 31.3 | 140 | 49.7 | 1.99 | 74.9 | 62.8 | 4.2 |
| 1.10 | SB | 28 | 0% | 25.0 | 140 | 42.9 | 1.08 | 104.4 | 35.8 | 3.0 |
| 1.11 | SB | 28 | 0% | 25.0 | 160 | 43.6 | 1.15 | 102.8 | 35.2 | 3.7 |
| 1.12 | SB | 28 | 0% | 25.0 | 180 | 44.1 | 1.17 | 97.5 | 35.7 | 5.0 |
| 2.1 | SB | 20 | 11% | 27.8 | 140 | 36.8 | 1.27 | 53.1 | 44.2 | 2.4 |
| 2.2 | SB | 20 | 11% | 27.8 | 160 | 36.2 | 1.27 | 52.5 | 62.1 | 2.9 |
| 2.3 | SB | 20 | 11% | 27.8 | 180 | 37.4 | 1.31 | 57.8 | 44.3 | 2.7 |
| 2.4 | SB | 20 | 25% | 31.3 | 180 | 39 | 1.55 | 53.4 | 56.6 | 2.4 |
| 2.5 | SB | 20 | 25% | 31.3 | 160 | 38 | 1.48 | 46.6 | 63.4 | 2.8 |
| 2.6 | SB | 20 | 25% | 31.3 | 140 | 38.8 | 1.46 | 39.7 | 30.4 | 2.3 |
| 2.7 | SB | 20 | 43% | 35.8 | 140 | 40.9 | 1.78 | 32.3 | 53.0 | 2.6 |
| 2.8 | SB | 20 | 43% | 35.8 | 160 | 41.4 | 1.82 | 35.7 | 77.2 | 2.7 |
| 2.9 | SB | 20 | 43% | 35.8 | 180 | 41.7 | 1.83 | 47.5 | 87.5 | 3.4 |
| 3.1 | SB | 38 | 25% | 31.3 | 180 | 62.2 | 2.52 | 97.3 | 64.8 | 2.2 |
| 3.2 | SB | 38 | 25% | 31.3 | 160 | 61 | 2.47 | 93.5 | 63.5 | 2.3 |
| 3.3 | SB | 38 | 25% | 31.3 | 140 | 60 | 2.32 | 83.9 | 68.2 | 2.4 |
| 3.4 | SB | 38 | 43% | 35.8 | 140 | 66.2 | 2.81 | 63.0 | 92.8 | 2.4 |
| 3.5 | SB | 38 | 43% | 35.8 | 160 | 65.4 | 2.81 | 78.6 | 86.5 | 2.3 |
| 3.6 | SB | 38 | 43% | 35.8 | 180 | 67.4 | 2.88 | 86.0 | 82.0 | 2.4 |
| 4.1 | None | 31.5 | 43% | 35.8 | 140 | 32.5 | 1.57 | 46.6 | 77.0 | 31.5 |
| 4.2 | None | 31.5 | 43% | 35.8 | 160 | 38.1 | 1.93 | 53.4 | 79.8 | 32.9 |
| 4.3 | None | 31.5 | 43% | 35.8 | 180 | 35.9 | 2.04 | 46.4 | 69.3 | 31.1 |
| 4.4 | None | 36.0 | 25% | 31.3 | 180 | 35.8 | 1.47 | 57.4 | 53.8 | 19.0 |
| 4.5 | None | 36.0 | 25% | 31.3 | 160 | 36.3 | 1.58 | 56.1 | 49.7 | 17.1 |
| 4.6 | None | 36.0 | 25% | 31.3 | 140 | 35.9 | 2.03 | 60.6 | 54.0 | 18.4 |
| 4.7 | None | 40.5 | 11% | 27.8 | 140 | 38.8 | 1.3 | 69.0 | 41.3 | 15.1 |
| 4.8 | None | 40.5 | 11% | 27.8 | 160 | 38.2 | 1.33 | 72.4 | 41.4 | 9.9 |
| 4.9 | None | 40.5 | 11% | 27.8 | 180 | 37.6 | 1.31 | 72.3 | 36.6 | 8.4 |
| 5.1 | None | 38.5 | 43% | 35.8 | 140 | 43.2 | 2.16 | 51.7 | 72.1 | 28.7 |
| 5.2 | None | 38.5 | 43% | 35.8 | 160 | 44.1 | 2.2 | 54.2 | 76.1 | 26.0 |
| 5.3 | None | 38.5 | 43% | 35.8 | 180 | 43.2 | 2.3 | 50.4 | 74.2 | 24.1 |
| 5.4 | None | 46.0 | 25% | 31.3 | 180 | 40.5 | 1.77 | 67.5 | 51.8 | 13.6 |
| 5.5 | None | 46.0 | 25% | 31.3 | 160 | 46.5 | 2.02 | 60.0 | 58.2 | 16.5 |
| 5.6 | None | 46.0 | 25% | 31.3 | 140 | 45.8 | 1.99 | 61.1 | 54.8 | 20.2 |
| 5.7 | None | 49.5 | 11% | 27.8 | 140 | 43.6 | 1.52 | 74.0 | 36.8 | 9.2 |
| 5.8 | None | 49.5 | 11% | 27.8 | 160 | 45 | 1.54 | 75.6 | 35.9 | 8.4 |
| 5.9 | None | 49.5 | 11% | 27.8 | 180 | 47 | 1.71 | 70.8 | 39.1 | 8.9 |

*Note for codes 4.1 to 5.9 the “Laminate” was a single layer structure as no support layer was present.

For Table 3, samples 6SL.1 through 6SL.6 were run on the same equipment under the same conditions as the samples in Table 2 with the aforementioned spunlace support layer while samples 6S.1 through 6S.4 were run with the aforementioned through-air bonded carded web support layer. The projection webs (“Card webs”) were made in the same fashion as those used in Table 2.

TABLE 3
Experimental parameters and test results code 6, alternative support layers.

| | | Card | | Card web | Texturizing | Laminate | Laminate | Laminate | Laminate | Laminate |
|---|---|---|---|---|---|---|---|---|---|---|
| | Support | web | | Speed V1 | Jet Press. | Weight | Thickness | MD Strength | Ext at Peak | MD Ext @10 N |
| CODE | layer | (gsm) | Overfeed | (m/min) | (bar) | (gsm) | (mm) | (N/50 mm) | Load MD (%) | (%) |
| 6SL.1 | SL | 28 | 25% | 31.3 | 180 | 82.6 | 2.19 | 107.5 | 23.6 | 1.9 |
| 6SL.2 | SL | 28 | 25% | 31.3 | 160 | 80 | 2.11 | 103.6 | 23.6 | 1.9 |
| 6SL.3 | SL | 28 | 25% | 31.3 | 140 | 81.1 | 2.07 | 101.5 | 20.2 | 1.8 |
| 6SL.4 | SL | 28 | 43% | 35.8 | 140 | 85.4 | 2.16 | 86.7 | 20.2 | 1.7 |
| 6SL.5 | SL | 28 | 43% | 35.8 | 160 | 84.2 | 2.53 | 93.4 | 20.8 | 1.6 |
| 6SL.6 | SL | 28 | 43% | 35.8 | 180 | 83.7 | 2.55 | 103.3 | 22.4 | 1.4 |
| 6S.1 | S | 28 | 25% | 31.3 | 180 | 68.2 | 2.56 | 89 | 56 | 4.2 |
| 6S.2 | S | 28 | 25% | 31.3 | 160 | 70 | 2.57 | 70 | 56.7 | 2.2 |
| 6S.3 | S | 28 | 25% | 31.3 | 140 | 72.5 | 2.71 | 67.7 | 62 | 2.8 |
| 6S.4 | S | 28 | 43% | 35.8 | 140 | 78 | 2.63 | 48.5 | 57.8 | 2.8 |

As can be seen in Tables 2 and 3, the key quality parameter of fabric thickness, which is a measure of the height of the projections as indicated by the thickness values, depended predominantly on the amount of overfeed of the projection web 16 into the texturizing zone 144. Relative to the data shown in Table 3, it can be seen that high overfeed ratios resulted in increased thickness. In addition, at the same overfeed ratios, higher fluid pressures resulted in higher thickness values, which in turn indicates an increased projection height. Table 3 shows the test results for samples made using alternative support layers. Codes 6S used a 40 gsm through-air bonded carded web and codes 6SL used a 52 gsm spunlaced material. These samples performed well and had good stability and appearance when compared to unsupported samples with no support layers.

FIG. 22 of the drawings depicts the sample thickness in millimeters relative to the percentage of projection web overfeed for a laminate (represented by a diamond) versus two samples that did not have a support layer (represented by a square and triangle). All reported values were an average of three samples. As can be seen from the data in FIG. 22, as overfeed was increased, the thickness of the sample also increased, showing the importance and advantage of using overfeed.

FIG. 23 of the drawings is a graph depicting the percentage of sample extension at a 10 Newton load relative to the amount of projection web overfeed for materials from Table 2. As can be seen from the graph in FIG. 23, when no support layer was present, there was a dramatic increase in the machine direction extensibility of the resultant sample as the percentage of overfeed of material into the texturizing zone was increased. In contrast, the sample with the spunbond support layer experienced virtually no increase in its extension percentage as the overfeed ratio was increased. This in turn resulted in the projection web having projections which are more stable during subsequent processing and which are better able to retain their shape and height.

As can be seen from the data and the graphs, higher overfeed and hence greater projection height also decreased the MD tensile strength and increased the MD extension at peak load. This was because the increased texturing provided more material (in the projections) that did not immediately contribute to resisting the extension and generating the load and allowed greater extension before the peak load was reached.

A key benefit of the laminate of both a projection web and a support layer compared to the single layer projection web with no support layer is that the support layer can reduce excessive extension during subsequent processing and converting which can pull out the fabric texture and reduce the height of the projections. Without the support layer 14 being integrated into the projection forming process, it was very difficult to form webs with projections that could continue to be processed without the forces and tensions of the process acting upon the web and negatively impacting the integrity of the projections, especially when low basis weight webs were desired. Other means can be used to stabilize the material such as thermal or adhesive bonding or increased entanglement but they tend to lead to a loss of fabric softness and an increased stiffness as well as increasing the cost. The fluid-entangled laminate web according to the present invention can provide softness and stability simultaneously. The difference between supported and unsupported textured materials is illustrated clearly in the last column of Table 2, which, for comparison, shows the extension of the samples at a load of 10N. The data is also displayed in FIG. 23 of the drawings. It can be seen that the sample supported by the spunbond support layer extended only a few percent at an applied load of 10 Newtons (N) and the extension was almost independent of the overfeed. In contrast the unsupported projection web extended by up to 30% at a 10 Newton load and the extension at 10N was strongly dependent on the overfeed used to texture the sample. Low extensions at 10N can be achieved for unsupported webs but only by having low overfeed, which results in low projection height, i.e., little texturing of the web.

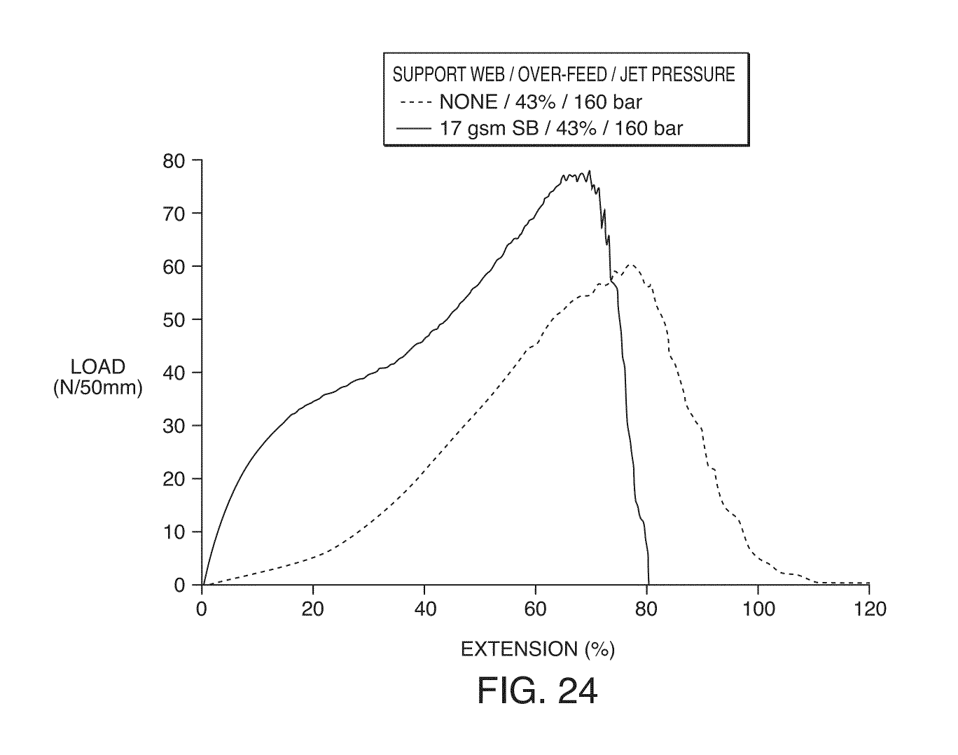

FIG. 24 of the drawings shows an example of the load-extension curves obtained in tensile testing of samples in the machine direction (MD) which is the direction in which highest loads are most likely to be experienced in winding up the material and in further processing and converting. The samples shown in FIG. 24 were all made using an overfeed ratio of 43% and had approximately the same areal density (45 gsm). It can be seen that the sample containing the spunbond support layer had a much higher initial modulus, the start of the curve was steep compared to that of the unsupported, single projection web by itself. This steeper initial part of the curve for the sample with the support layer was also recoverable as the sample was elastic up to the point where the gradient started to decrease. The unsupported sample had a very low modulus and permanent deformation and loss of texture occurred at a lower load. FIG. 24 of the drawings shows the load-extension curves for both a supported and unsupported fabric. Note the relative steepness of the initial part of the curve for the supported fabric/laminate according to the present invention. This means that the unsupported sample is relatively easily stretched and a high extension is required to generate any tension in it compared to the supported sample. Tension is often required for stability in later processing and converting but the unsupported sample is more likely to suffer permanent deformation and loss of texture as a result of the high extension needed to maintain tension.

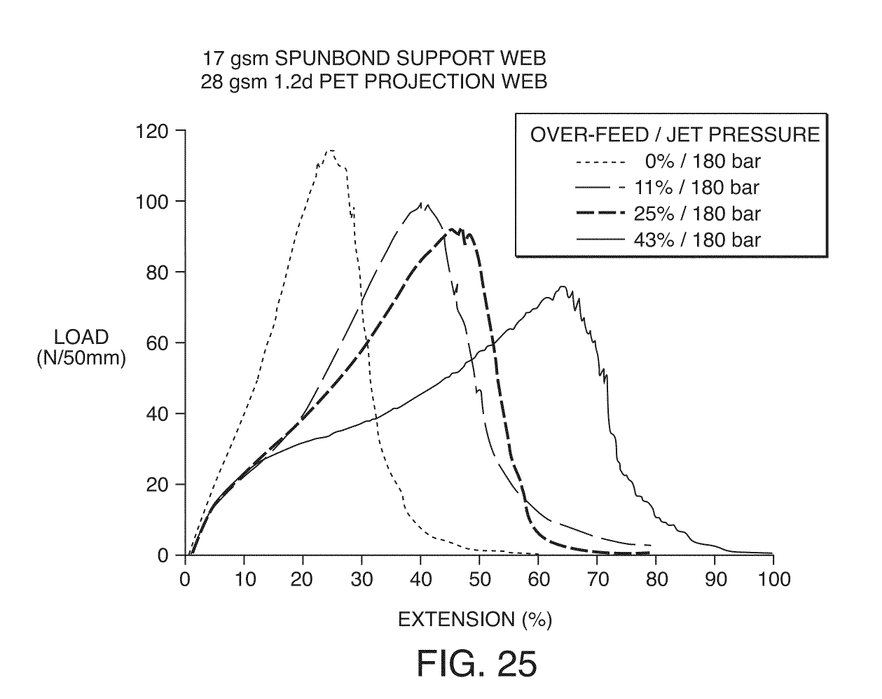

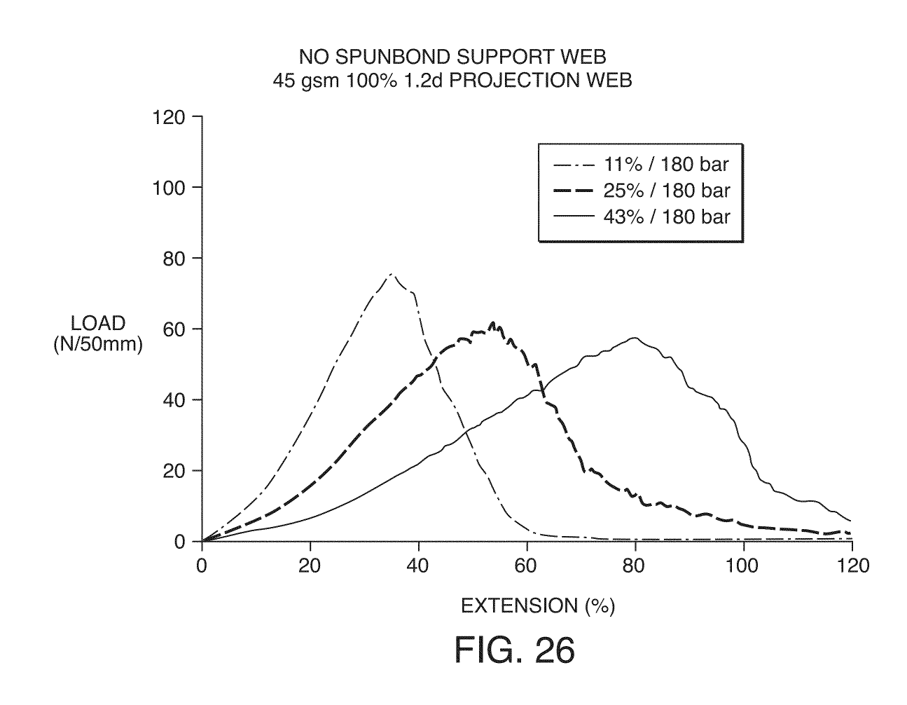

FIGS. 25 and 26 of the drawings show a set of curves for a wider range of conditions. It can be seen that the samples with a low level of texturing from low overfeed were stiffer and stronger (despite being slightly lighter) but the absence of texture rendered them not useful in this context.

All supported laminate samples according to the present invention had higher initial gradients compared to the unsupported samples.

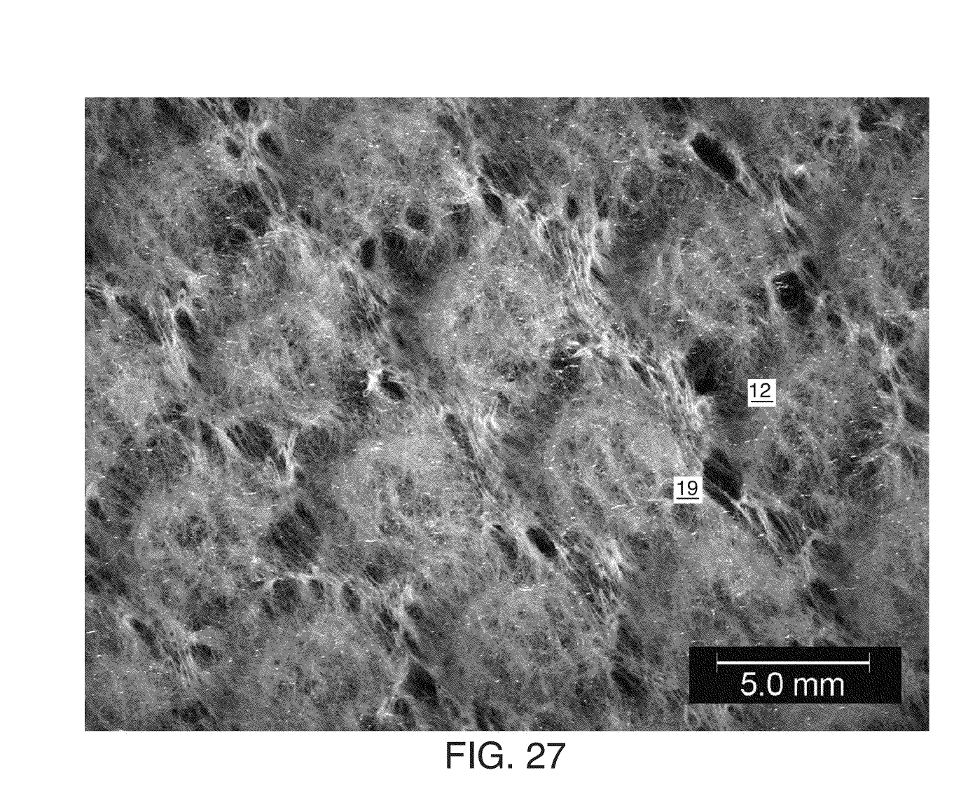

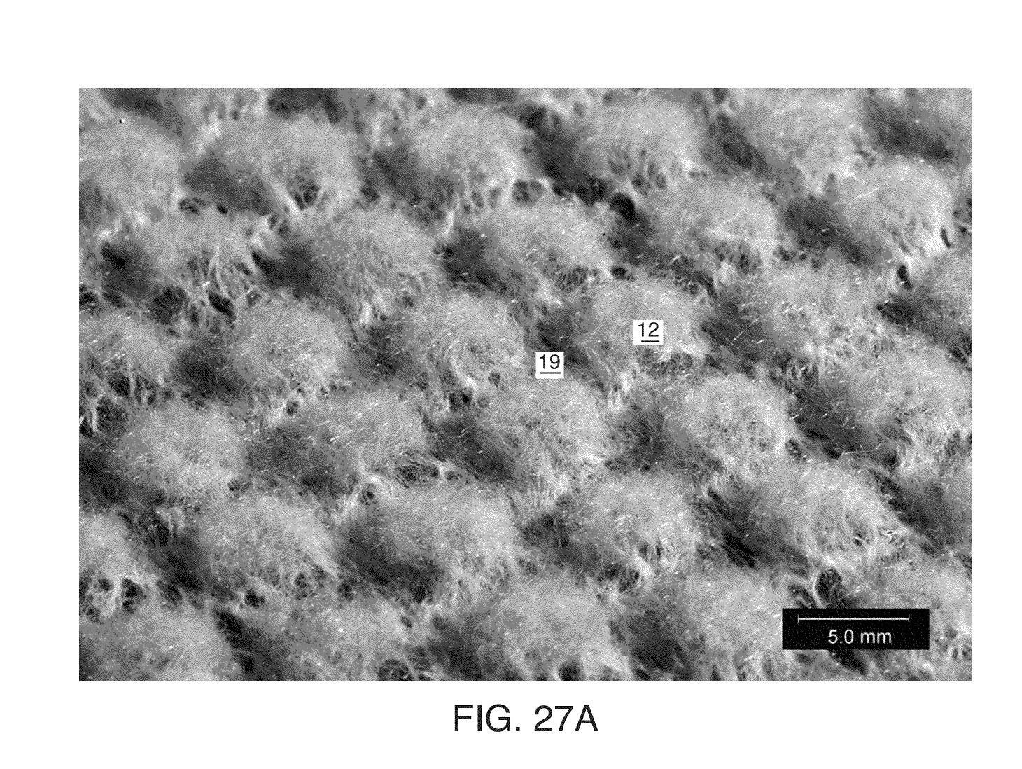

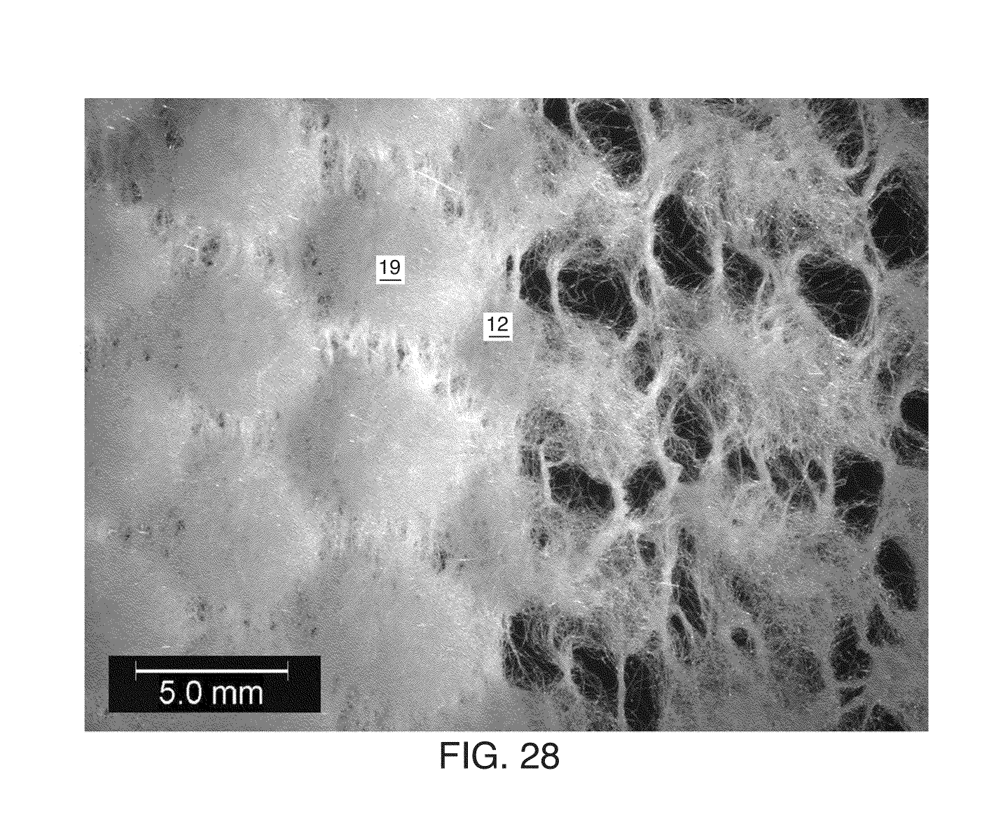

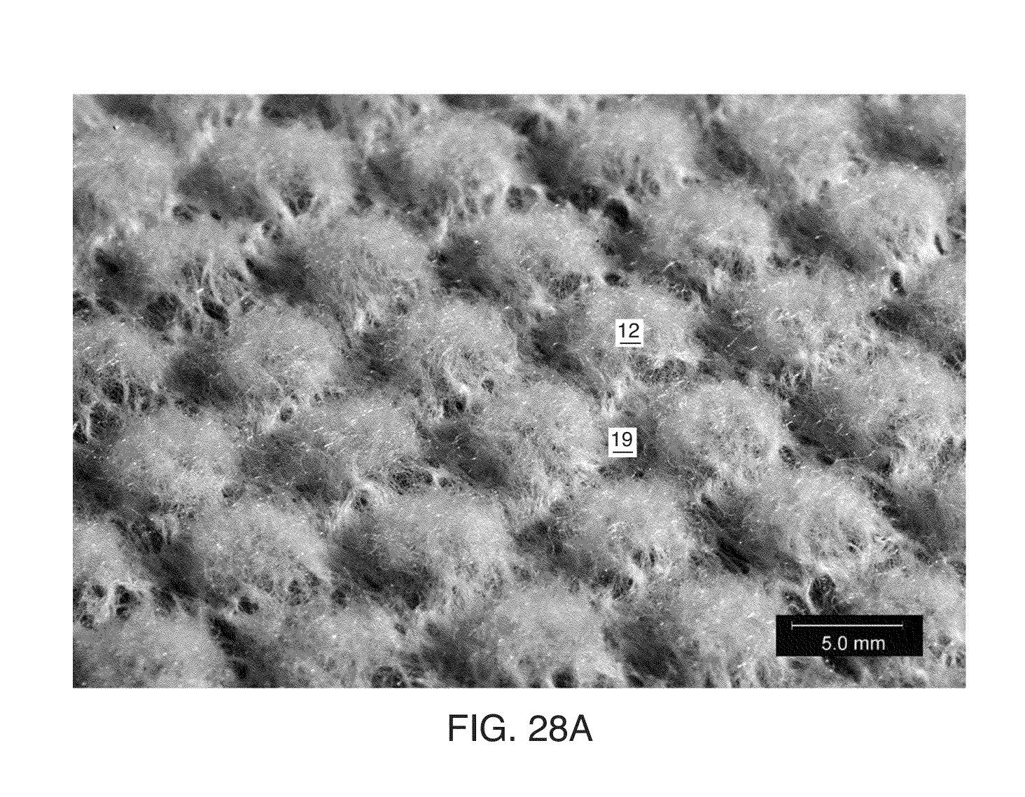

The level of improvement in the overall quality of the fluid-entangled laminate web 10 as compared to a projection web 16 with no support layer 14 can be seen by comparing the photos of the materials shown in FIGS. 27, 27A, 28, 28A and 29. FIGS. 27 and 27A are photos of the sample represented by Code 3-6 in Table 2. FIGS. 28 and 28A are photos of the sample represented by Code 5-3 in Table 2. These codes were selected as they both had the highest amount of overfeed (43%), and jet pressure (180 bar) using comparable projection web basis weights (38 gsm and 38.5 gsm respectively) and thus the highest potential for good projection formation. As can be seen by the comparison of the two codes and accompanying photos, the supported web/laminate formed a much more robust and visually discernible projections and uniform material than the same projection web without a support layer. It also had better properties as shown by the data in Table 2. As a result, the supported laminate according to the present invention is much more suitable for subsequent processing and use in such products as, for example, personal care absorbent articles.

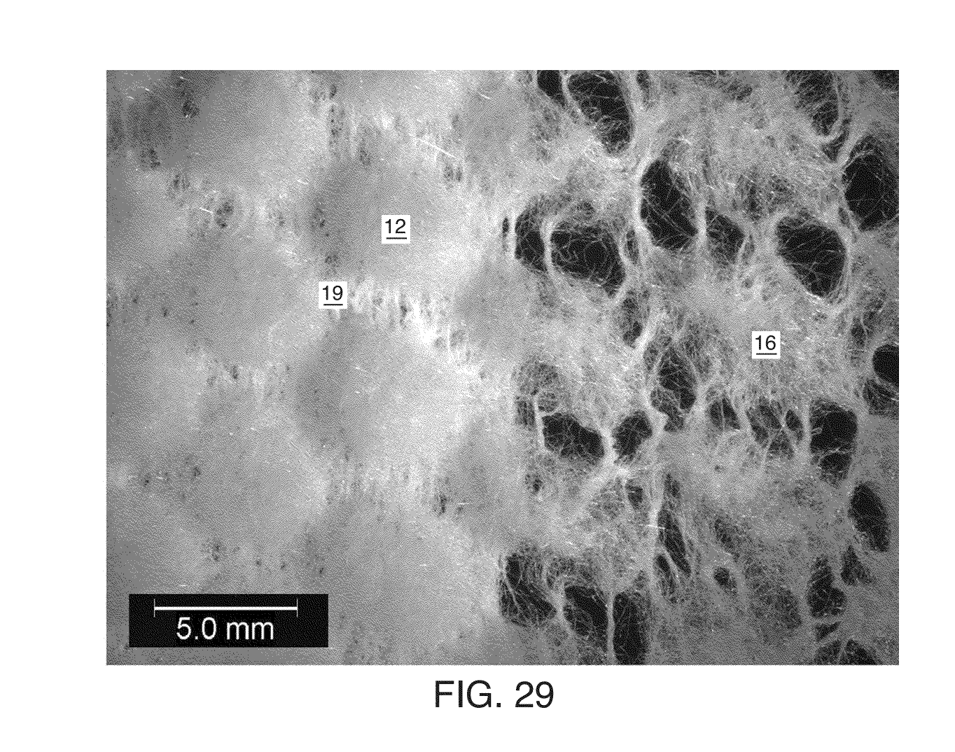

FIG. 29 is a photo at the interface of a projection web with and without a support layer. As can be seen in this photo, the supported projection web has a much higher level of integrity. This is especially important when the material is to be used in such end applications as personal care absorbent articles where it is necessary (often with the use of adhesives) to attach the projection web to subjacent layers of the product. With the unsupported projection web, adhesive bleed through is a much higher threat. Such bleed through can result in fouling of the processing equipment and unwanted adhesion of layers, thereby causing excessive downtime with manufacturing equipment. In use, the unsupported projection web is more likely to allow absorbed fluids taken in by the absorbent article (such as blood, urine, feces and menses) to flow back or “rewet” the top surface of the material, thereby resulting in an inferior product.

Another advantage evident from visual observation of the samples (not shown) was the coverage and the degree of flatness of the back of the first surface 18 on the external side of the support layer 14 and thus the laminate 10 resulting from the formation process when compared to the inner surface 24 of a projection web 16 run through the same process 100 without a support layer 14. Without the support layer 14, the external surface of the projection web 16 opposite the projections 12 was uneven and relatively non-planar. In contrast, the same external surface of the fluid-entangled laminate web 10 according to the present invention with the support layer 14 was smoother and much flatter. Providing such flat surfaces improves the ability to adhere the laminate to other materials in later converting. As noted in the exemplary product embodiments described below, when fluid-entangled laminate webs 10 according to the present invention are used in such items as personal care absorbent articles, having flat surfaces which readily interface with adjoining layers is important in the context of joining the laminate to other surfaces so as to allow rapid passage of fluids through the various layers of the product. If good surface-to-surface contact between layers is not present, fluid transfer between the adjoining layers can be compromised.

### Example 2

To demonstrate the efficacy of the fluid-entangled laminate web 10 as a female component 268 of a mechanical fastening system, a series of fluid-entangled laminate webs 10 were compared with a pattern-unbonded nonwoven material such as is commonly used as a female component 268 of mechanical fastening systems. The series of fluid-entangled laminate webs 10 have the material descriptions as found in Table 4 below and are available from Textor Technologies PTY LTD of Tullamarine, Australia. The pattern-unbonded nonwoven web is also described in Table 4 below.

TABLE 4
Material Descriptions

| Material Code | Material Description |
|---|---|
| A | Fluid-Entangled Laminate Web: A dual layer fluid-entangled laminate web |
| | having 1) a support layer of 17 gsm polypropylene point bonded web made from |
| | 1.8 denier polypropylene spunbond fibers which were subsequently point bonded |
| | with an overall bond area per unit area of 17.5% made by Kimberly-Clark |
| | Australia of Milsons Point, Australia and 2) a projection layer of 38 gsm carded |
| | staple fiber web made from 100% 1.2 denier, 38 mm long polyester staple fibers |
| | available from the Huvis Corporation of Daejeon, Korea. The projection layer has |
| | about 4.4% open area in the land areas and has less than about 0.2% open area in |
| | the projections. The projection layer has a projection diameter of about 4 mm. |
| | The web is made wettable with up to about 0.3% of 50:50 ratio of Ahcovel/SF-19 |
| | on the bottom of the support layer and up to about 0.12% of Ahcovel on the top of |
| | the projection layer. The web has a thickness of 2.4 mm when measured under a |
| | pressure of 0.345 kPa. The web has a total basis weight of 55 gsm. The web is |
| | available from Textor Technologies PTY LTD of Tullamarine, Australia. |
| B | Fluid-Entangled Laminate Web: A dual layer fluid entangled laminate web having |
| | 1) a support layer of 10 gsm polypropylene point bonded web made from 1.8 |
| | denier polypropylene spunbond fibers which were subsequently point bonded with |
| | an overall bond area per unit area of 17.5% made by Kimberly-Clark Australia of |
| | Milsons Point, Australia and 2) a projection layer of 38 gsm carded staple fiber |
| | web made from 100% 1.2 denier, 38 mm long polyester staple fibers available |
| | from the Huvis Corporation of Daejeon, Korea. The projection layer has about |
| | 8.4% open area in the land areas and has less than about 0.1% open area in the |
| | projections. The projection layer has a projection diameter of about 4 mm. The |
| | web is made wettable with up to about 0.3% of 50:50 ratio of Ahcovel/SF-19 on |
| | the bottom of the support layer and up to about 0.12% of Ahcovel on the top of |
| | the projection layer. The web has a thickness of 2.4 mm when measured under a |
| | pressure of 0.345 kPa. The web has a total basis weight of 48 gsm. The web is |
| | available from Textor Technologies PTY LTD of Tullamarine, Australia. |
| C | Fluid-Entangled Laminate Web: A dual layer fluid-entangled laminate web having |
| | 1) a support layer of 10 gsm polypropylene point bonded web made from 1.8 |
| | denier polypropylene spunbond fibers which were subsequently point bonded with |
| | an overall bond area per unit area of 17.5% made by Kimberly-Clark Australia of |
| | Milsons Point, Australia and 2) a projection layer of 38 gsm carded staple fiber |
| | web made from 100% 1.2 denier, 38 mm long polyester staple fibers available |
| | from the Huvis Corporation of Daejeon, Korea. The projection layer has about |
| | 18.5% open area in the land areas and has less than about 0.5% open area in the |
| | projections. The projection layer has a projection diameter of about 4 mm. The |
| | web is made wettable with up to about 0.3% of 50:50 ratio of Ahcovel/SF-19 on |
| | the bottom of the support layer and up to about 0.12% of Ahcovel on the top of |
| | the projection layer. The web has a thickness of 2.3 mm when measured under a |
| | pressure of 0.345 kPa. The web has a total basis weight of 48 gsm. The web is |
| | available from Textor Technologies PTY LTD of Tullamarine, Australia. |
| D | Fluid-Entangled Laminate Web: A dual layer fluid-entangled laminate web |
| | having 1) a support layer of 10 gsm polypropylene point bonded web made from |
| | 1.8 denier polypropylene spunbond fibers which were subsequently point bonded |
| | with an overall bond area per unit area of 17.5% made by Kimberly-Clark |
| | Australia of Milsons Point, Australia and 2) a projection layer of 38 gsm carded |
| | staple fiber web made from 100% 1.2 denier, 38 mm long polyester staple fibers |
| | available from the Huvis Corporation of Daejeon, Korea. The projection layer has |
| | greater than about 20% open area in the land areas and has less than about 1% |
| | interstitial fiber-to-fiber spacing in the projections. The projection layer has a |
| | projection diameter of about 4 mm. The web is made wettable with up to about |
| | 0.3% of 50:50 ratio of Ahcovel/SF-19 on the bottom of the support layer and up to |
| | about 0.12% of Ahcovel on the top of the projection layer. The web has a |
| | thickness of 2.1 mm when measured under a pressure of 0.345 kPa. The web has |
| | a total basis weight of 48 gsm. The web is available from Textor Technologies |
| | PTY LTD of Tullamarine, Australia. |
| E | Pattern-Unbonded Nonwoven Web: 59 gsm pattern-unbonded nonwoven web, |
| | bicomponent spunbond of high density polyethylene and polypropylene in a 50:50 |
| | ratio, bonded with a point unbonded pattern, as described in U.S. Pat. No. |
| | 5,858,515 to Stokes et al., which is incorporated herein in its entirety by reference |
| | thereto for all purposes. |
| F | Male Component: This hook material includes hook elements having an average |
| | overall height measured from the top surface of the base material to the highest |
| | point on the hook elements. The average height of the hook elements used in |
| | conjunction with the present invention is about 0.012 inches. This hook material |
| | has a hook density of about 392 hooks per square centimeter. The thickness of the |
| | hook base material is about 0.004 inches. This hook material is available from |
| | Velcro U.S.A. as CFM-85-1470. |

The thickness of the materials A-D set forth in Table 4 above were measured using a Mitutoyo model number IDF-1050E thickness gauge with a foot pressure of 345 Pa (0.05 psi). Measurements were taken at room temperature (about 20 degrees Celsius) and reported in millimeters using a round foot with a diameter of 76.2 mm (3 inches).

The tensile strength of the materials, defined as the peak load achieved during the test, was measured in the Machine Direction (MD) according to the Method to Determine Tensile Strength as described herein to provide a MD peak load. The peak stretch in the Machine Direction was also evaluated according to the Method to Determine Tensile Strength described herein. The peel strength and the shear strength of the materials, which can provide an understanding of how well each material can function as a female component 268 of a mechanical fastening system of an absorbent article, was measured according to the Method to Determine Peel Strength and the Dynamic Shear Strength Test Method described herein. When determining the peel strength and the shear strength, the tests were performed using a single type of male component for a mechanical fastening system, described in Table 4 as Material F. For each of the measurements of tensile strength, peak stretch, peel strength and shear strength, for each material evaluated, ten samples of that material were evaluated and the average is presented in Table 5 below, as well as the standard deviation.

The percent void space was evaluated for the materials according to the Method to Determine Percent Void Space described herein. As described herein, the percentage of void space can provide an evaluate of the amount of empty space in the z-plane of a fibrous structure such as, for example, a projection 12 of a fluid-entangled laminate web 10. The percentage of void space is different from the percentage of open area as the percentage of open area can provide an evaluation of the open space where light can pass through a fibrous material in the x-y plane. For each material evaluated, six samples of that material were evaluated and the average is present in Table 5 as well as the standard deviation.

Additionally, the orientation of the materials was evaluated. The field orientation (“anisotropy”) as well as fiber segment orientation (“feature horizontal/vertical projection”) for each material sample was evaluated. The field orientation is the overall orientation of the material sample and the fiber segment orientation is the orientation of individual segments of fibers in the material sample. The orientations were determined according to the Method to Determine Orientation described herein. The percent rotational relative standard deviation was also calculated for each of the samples. For each of the materials evaluated, six samples of that material were evaluated and the average is present in Table 5 as well as the standard deviation.

The following Table (Table 5) summarizes the test results. Where a value is not present in Table 5 for a particular parameter for a particular material, that material was not tested for that parameter.

TABLE 5
Experimental Results

| | Code | | | | |
|---|---|---|---|---|---|
| | A | B | C | D | E |
| MD Peak Load | | 3470.0 | 4415.7 | 5015.4 | 2872.4 |
| (gf per inch) | | | | | |
| MD Peak Load STD | | 211.5 | 315.4 | 497.7 | 438.9 |
| MD Peak Stretch (%) | | 90.9 | 78.26 | 87.1 | 18.7 |
| MD Peak Stretch STD | | 4.7 | 7.9 | 10.3 | 5.7 |
| Peel Strength (gf) | 157.2 | 433.3 | 241.0 | 338.4 | 135.5 |
| Peel Strength STD | 66.9 | 292.5 | 63.9 | 128.2 | 36.6 |
| Shear Strength (gf) | 3525.7 | 2469.2 | 3669.3 | 4451.8 | 4649.0 |
| Shear Strength STD | 513.1 | 147.2 | 161.2 | 335.1 | 432.1 |
| Void Space (%) | 74.9 | 74.6 | 75.0 | | 52.9 |
| Void Space STD | 3.2 | 2.3 | 2.4 | | 3.1 |
| Field Orientation | | 0.95 | 0.98 | 0.94 | 1.85 |
| (Anisotropy) | | | | | |
| Field Orientation STD | | 0.03 | 0.05 | 0.06 | 0.27 |
| Field Orientation | | 6.3 | 5.0 | 8.2 | 59.3 |
| Rotational % RSD | | | | | |
| Fiber Segment | | 1.61 | 1.71 | 1.65 | 3.93 |
| Orientation (Feat. | | | | | |
| Horz./Vert. Proj.) | | | | | |
| Fiber Segment | | 0.12 | 0.08 | 0.19 | 0.50 |
| Orientation STD | | | | | |
| Fiber Segment | | 10.5 | 9.4 | 13.1 | 78.2 |
| Orientation | | | | | |
| Rotational % RSD | | | | | |
| (Feature Horz./Vert. | | | | | |
| Proj. Rotational % | | | | | |
| RSD) | | | | | |

As can be seen in Table 5, while the pattern-unbonded nonwoven web (Material E) had a higher basis weight than the fluid-entangled laminate webs (Materials B-D), the fluid-entangled laminate webs, Materials B-D, had a greater tensile strength in the machine direction (MD peak load) than the pattern-unbonded nonwoven (Material E). An advantage of the fluid-entangled laminate webs 10 over the pattern-unbonded nonwoven material can be the requirement for less fibrous material to manufacture the fluid-entangled laminate webs while still maintaining machine direction strength.

Table 5 also shows that the tensile strength in the machine direction (MD peak load) increases as the percentage of open area in the land area 19 in a given area of the fluid-entangled laminate web 10 increases. As described herein, the fluid-entangled laminate webs 10 are formed utilizing a fluid-entanglement process and the pressure or dwell times of the impinging fluid-entangling jets can be changed during the entangling process to effect a change on the resultant fluid-entangled laminate web 10, such as, for example, increasing hole sizes which can, thereby, increase the percentage of open area. Increasing the fluid-entangling pressure during the fluid-entangling process can cause the fibers in the land areas 19 to shift, thereby, increasing the spacing between the fibers (e.g., increasing the open area). Without being bound by theory, it is believed that the fibers which have shifted can form bundles of fibers surrounding the larger open areas and it is believed that the fibers can also bundle at the base of the projections 12 in the fluid-entangled laminate web. It is believed that the bundles of fibers can increase the strength of the fluid-entangled laminate web 10 in the machine direction. It is believed, therefore, that the machine direction strength of the fluid-entangled laminate web 10 is not disadvantaged by an increase in the percentage of open area in the land area 19 in a given area of the fluid-entangled laminate web 10 and some additional advantages of the increase in the percentage of open area in the land area 19 in a given area of the fluid-entangled laminate web 10 can be that as the percentage of open area increases, the fluid-entangled laminate web 10 can appear softer and can feel softer.

As indicated in Table 1, the peak stretch of the fluid-entangled laminate webs (Materials B-D) is greater than the peak stretch of the pattern-unbonded nonwoven (Material E). The fluid-entangled laminate webs 10 are, as described herein, manufactured via fluid-entanglement processes while the pattern-unbonded nonwoven undergoes a thermal bonding process which is different from the fluid-entangling process of the current document. Without being bound by theory, it is believed that the thermal bonding process of the pattern-unbonded nonwoven, which bonds the fibers more firmly in place when compared to the fluid-entanglement processes described herein, can result in a decrease in the stretch capability of the pattern-unbonded nonwoven web.

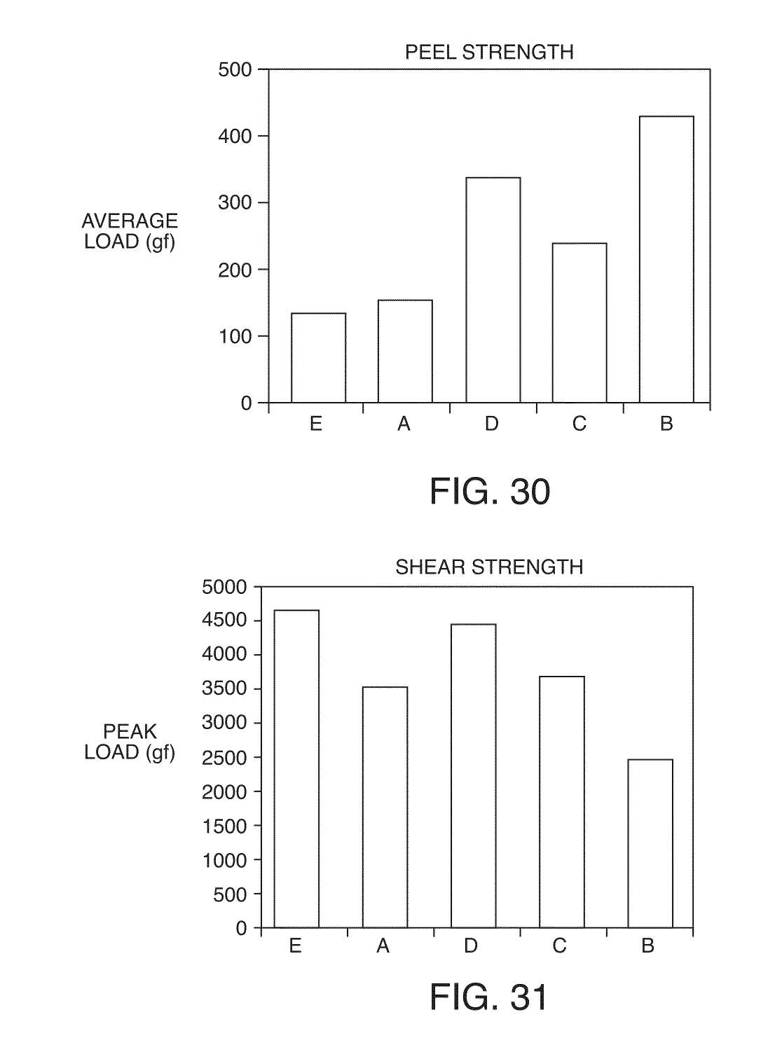

As indicated in Table 5, and as illustrated in FIG. 30, the peel strength of the fluid-entangled laminate webs (Materials A-D) is greater than the peel strength of the pattern-unbonded nonwoven (Material E). The fluid-entangled laminate webs (Materials A-D) contain discontinuous fibers which are not present in the pattern-unbonded nonwoven web. The fluid-entangled laminate webs 10 are also, as described herein, manufactured via fluid-entanglement processes while the pattern-unbonded nonwoven web undergoes a thermal bonding process which is different from the fluid-entangling process of the current document. Without being bound by theory, it is believed that the thermal bonding process of the pattern-unbonded nonwoven, which bonds the fibers more firmly in place when compared to the fluid-entanglement processes described herein, can result in a decrease in the stretch capability of the pattern-unbonded nonwoven web which can, therefore, result in an increase in the breakage of fibers of the pattern-unbonded nonwoven during the peeling process. The early breakage of the fibers can result in a decrease in the peel strength of the pattern-unbonded nonwoven web. In contrast, the fluid-entanglement processes described herein can result in a more loose entanglement of the fibers and, therefore, the fibers can still move and/or stretch during the peeling process allowing for an increase in the peel strength and an increase in the percentage of stretch of the fluid-entangled laminate webs 10.

As indicated in Table 5, and as illustrated in FIG. 31, the shear strength of the fluid-entangled laminate webs (Materials A-D) is comparable, or only slightly lower than, the shear strength of the pattern-unbonded nonwoven web (Material E). A review of Table 5 and FIGS. 30 and 31 can provide that the fluid-entangled laminate webs (Materials A-D) can have greater peel strength with comparable, or only slightly lower, shear strength when utilized as a female component 268 of a mechanical fastening system when compared with the pattern-unbonded nonwoven web (Material E). As noted above, the basis weights of the fluid-entangled laminate webs (Materials A-D) are lower than the basis weight of the pattern-unbonded nonwoven and, therefore, less fibrous material is needed to manufacture the fluid-entangled laminate webs (Materials A-D) while providing fluid-entangled laminate webs 10 that will have better peel strength and comparable shear strength to materials which are currently utilized as a female component 268 of a mechanical fastening system.

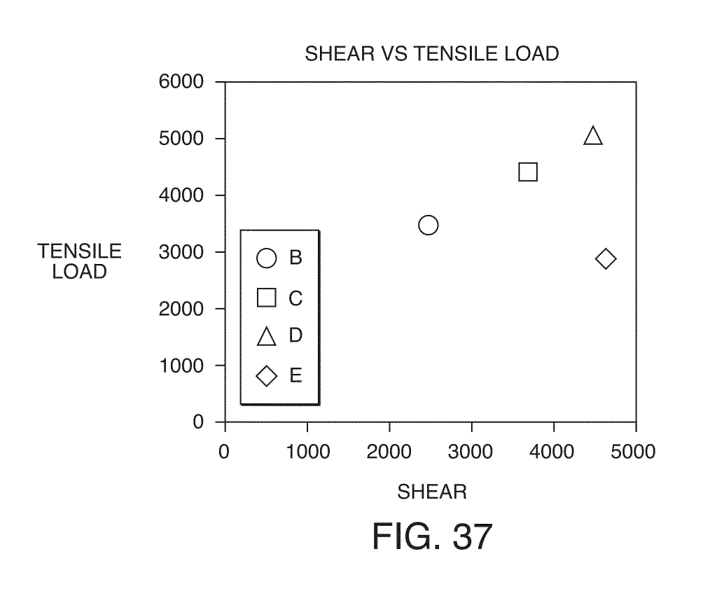

As illustrated in FIG. 37, which is a comparison of the shear strength of the materials (Materials B-E) versus tensile load of the materials (Materials B-E), as the tensile load in the machine direction (MD peak load) increases for the fluid-entangled laminate webs (Materials B-D), the shear strength also increases for the fluid-entangled laminate webs (Materials B-D). Additionally, as illustrated in FIG. 37, as the percentage of open area in the land areas 19 of the fluid-entangled laminate webs 10 increases, the shear strength and the tensile load in the machine direction also increase. Without being bound by theory, as discussed above, it is believed that increasing the dwell time or pressure of the impinging entangling jets in the fluid-entanglement processes described herein, causes the fibers to shift and form bundles of fibers at the base of the projections 12 of the fluid-entangled laminate webs 10 and/or surrounding larger open areas. As noted above, it is believed that it is the bundling of the fibers which contributes to the tensile strength of the fluid-entangled laminate webs 10 as represented by the increase in the tensile load in the machine direction (MD peak load). Additionally, it is believed that the male component of a mechanical fastening system, such as hooks, can catch and engage the bundles of fibers during shear which can be represented by the increase in shear strength.

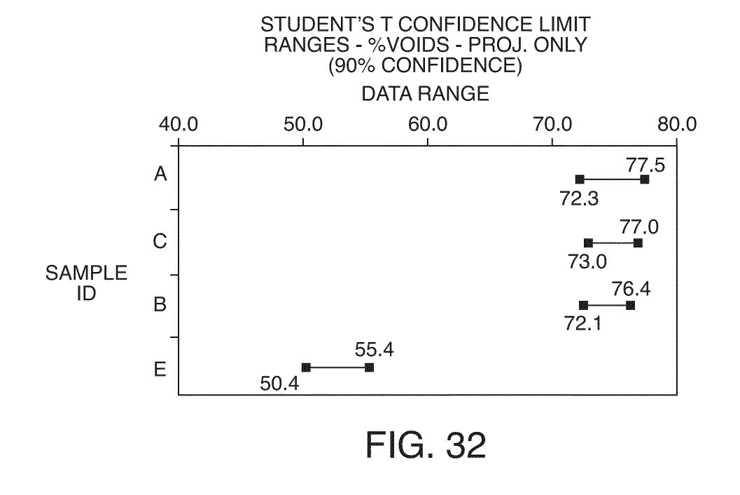

As indicated in Table 5, and as illustrated in FIG. 32, the projections of the fluid-entangled laminate webs (Materials A-C) had a greater percentage of void space than the raised areas of the pattern-unbonded nonwoven web. When viewing FIGS. 30, 31, and 32, it can be seen that the fluid-entangled laminate webs 10 have a greater percentage of void space in the projections 12, a greater peel strength and a comparable, or slightly lower, shear strength when compared with the pattern-unbonded nonwoven (Material E). Without being bound by theory, it is believed that the greater void space percentage in the projections 12 of the fluid-entangled laminate webs 10 can provide more open area in the Z-direction of the projections 12 of the fluid-entangled laminate web 10 to allow for a male component (such as hooks) to catch and engage the fibers of the fluid-entangled laminate web 10.

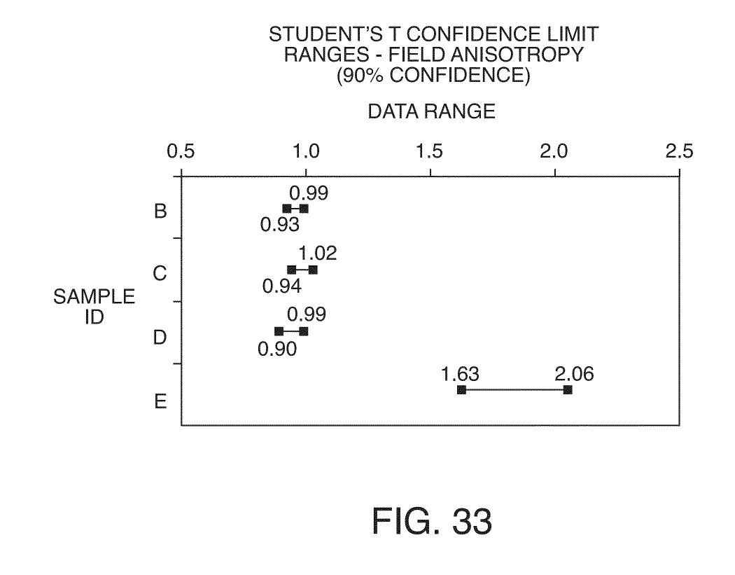

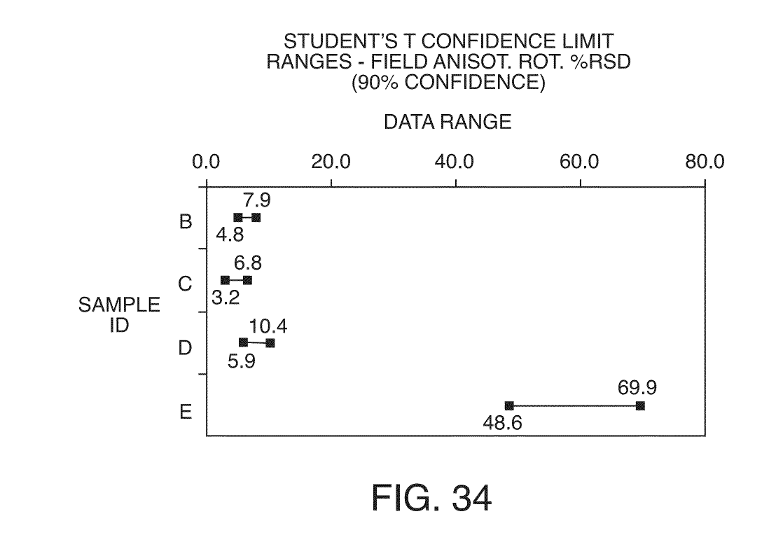

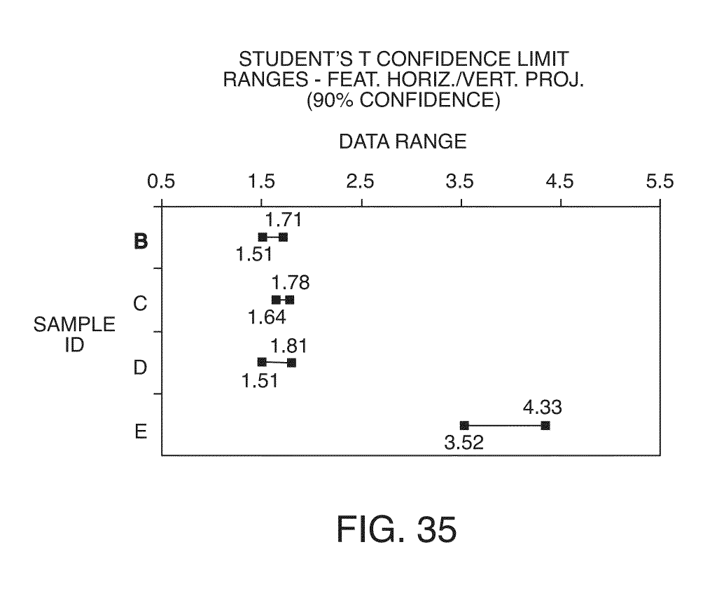

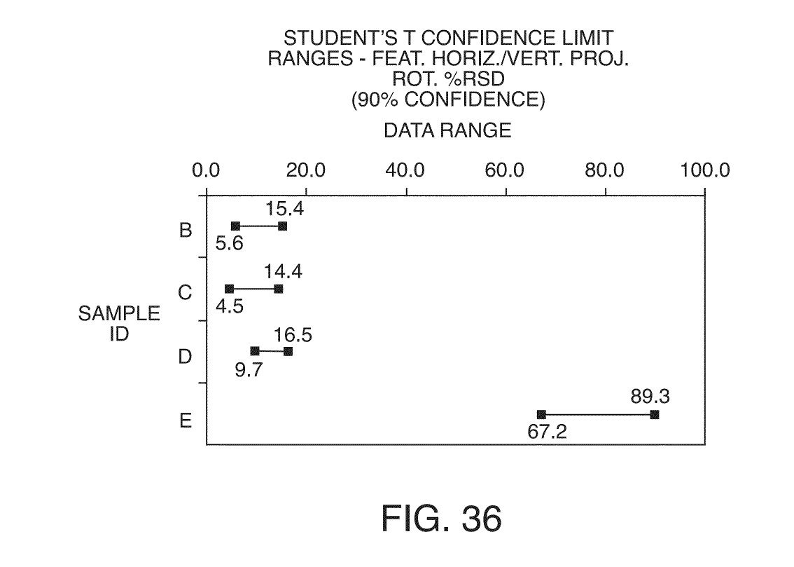

As indicated in Table 5, and as illustrated in FIGS. 33-36, the field orientation and the field orientation rotational percent relative standard deviation (FIGS. 33 and 34) and the fiber segment orientation and the fiber segment orientation rotational percent relative standard deviation (FIGS. 35 and 36) of the fluid-entangled laminate webs (Materials B-D) demonstrate that the fluid-entangled laminate webs (Materials B-D) have a lower degree of orientation than the pattern-unbonded nonwoven (Material E). With regards to the field orientation, assuming the machine direction is known during the image acquisition phase, materials which have values greater than 1 are more oriented in the machine direction and materials with orientation values less than 1 are more oriented in the cross direction. Additionally, with regard to the field orientation, materials with orientation values of about 1 are random in their orientation. As illustrated in FIG. 33, the fluid-entangled laminate webs (Materials B-D) had anisotropy values ranging from 0.9 to 1.02 (0.93-0.98 for Material B, 0.94-1.02 for Material C, and 0.90-0.99 for Material D) indicating a random field orientation. The pattern-unbonded nonwoven web (Material E) had anisotropy values ranging from 1.63-2.06 indicating that the pattern-unbonded nonwoven web had a field orientation in the machine direction. Additionally, as described above, the percent relative standard deviation across rotation values can indicate whether a material has a random orientation or whether the material is more oriented in the machine direction or cross direction. As described above, a material which has a random orientation will have a lower percent relative standard deviation across rotation values when compared with a material having greater fiber orientation. As can be seen in FIG. 34, the fluid-entangled laminate webs (Materials B-D) each have a field orientation rotational percent relative standard deviation less than 20% while the pattern-unbonded nonwoven web (Material E), in comparison, has a field orientation rotational percent relative standard deviation greater than 20%, and is greater than 40%. The pattern-unbonded nonwoven web (Material E), therefore, has a higher field orientation than any of the fluid-entangled laminate webs (Materials B-D).

With regard to the orientation of segments of fibers of each of the materials evaluated, a higher value observed for a fiber segment orientation (Feat. Horiz./Vert. Proj.) will provide an indication that the fiber segment orientation is more oriented in the machine direction while a lower value observed for a fiber segment orientation (Feat. Horiz./Vert. Proj.) will provide an indication that the fiber segment orientation is more random or, if low enough, more cross-direction oriented. This concept is further illustrated by reviewing the Feat. Horiz/Vert Proj. rotational percent relative standard deviation. As described above, a fiber which has a random orientation will have a lower rotational percent relative standard deviation than a fiber which is more oriented, such as in the machine direction. As can be seen in FIG. 35, the fluid-entangled laminate webs (Materials B-D) each have a lower fiber segment orientation (and, therefore, higher random orientation) when compared with the pattern unbonded nonwoven web. As further illustrated in FIG. 36, the fluid-entangled laminated webs (Materials B-D) each have a fiber segment orientation rotational percent relative standard deviation less than 20% while the pattern unbonded nonwoven web (Material E), in comparison, has a fiber segment orientation greater than 20%, and is greater than 60%. The pattern unbonded nonwoven web (Material E), therefore, has a higher fiber segment orientation than any of the fluid-entangled laminate webs (Materials B-D).

The pattern-unbonded nonwoven web (Material E) can have a higher shear strength than the fluid-entangled laminate webs (Materials B-D) due to the higher orientation of the fibers in the pattern-unbonded nonwoven, but the fluid-entangled laminate webs 10, with the lower degree of orientation (i.e., higher degree of randomness) can have a higher percentage of void space for the male component (e.g., hooks) of a mechanical fastening system to engage which increases the capability of the male component to engage with the female component 268. A higher engagement between the male component and the fluid-entangled laminate web 10, as the female component 268, can result in higher peel strength and a comparable, or slightly lower, shear strength than the pattern unbonded nonwoven. The random orientation of the fibers of the fluid-entangled laminate webs 10 can also increase the flexibility in the placement of the ears (and, therefore, the male component) of the absorbent article 200 by a user as the random orientation of the fibers of the fluid-entangled laminate webs 10 can provide an increase in the flexibility of the angle at which the ears (and, therefore, the male component) are engaged with the fluid-entangled laminate webs.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a. a bodyside liner;
b. a backsheet comprising a garment facing surface;
c. an absorbent core positioned between the bodyside liner and the backsheet;
d. a female component of a mechanical fastening system positioned on the garment facing surface of the backsheet, the female component comprising a fluid-entangled laminate web, the fluid-entangled laminate web comprising:
i. a support layer comprising opposed first and second surfaces;
ii. a projection layer comprising a plurality of fibers and opposed inner and outer surfaces, the second surface of the support layer in contact with the inner surface of the projection layer; and
iii. a plurality of hollow projections formed from a first plurality of the plurality of fibers in the projection layer, the plurality of hollow projections extending from the outer surface of the projection layer in a direction away from the support layer.

2. The absorbent article of claim 1 wherein a second plurality of fibers of the plurality of fibers in the projection layer are entangled with the support layer.

3. The absorbent article of claim 1 wherein the projections have a height greater than about 1 mm.

4. The absorbent article of claim 1 wherein the fluid entangled laminate web further comprises a land area which has greater than about 4% open area in a chosen area of the fluid entangled laminate web.

5. The absorbent article of claim 1 wherein the fluid-entangled laminate web further comprises a peak load in the machine direction of greater than about 3000 gf per inch.

6. The absorbent article of claim 1 wherein the fluid-entangled laminate web further comprises a fiber segment orientation rotational percent relative standard deviation of less than about 20%.

7. The absorbent article of claim 1 wherein the fluid-entangled laminate web further comprises a field anisotropy rotational percent relative standard deviation of less than about 20%.

8. The absorbent article of claim 1 wherein the fluid-entangled laminate web further comprises a peel strength greater than about 150 gf.

9. The absorbent article of claim 1 wherein the fluid-entangled laminate web further comprises a percentage of void space greater than about 60%.

10. The absorbent article of claim 1 wherein the fluid-entangled laminate web has a basis weight of less than about 58 gsm.

11. The absorbent article of claim 1 wherein the fluid-entangled laminate web has a peak stretch in the machine direction greater than about 20%.

12. An absorbent article comprising:
a. a bodyside liner;
b. a backsheet comprising a garment facing surface;
c. an absorbent core positioned between the bodyside liner and the backsheet;
d. a female component of a mechanical fastening system positioned on the garment facing surface of the backsheet, the female component comprising a fluid-entangled laminate web, the fluid-entangled laminate web comprising:
i. a support layer comprising opposed first and second surfaces;
ii. a projection layer comprising a plurality of fibers and opposed inner and outer surfaces, the second surface of the support layer in contact with the inner surface of the projection layer;
iii. a plurality of hollow projections formed from a first plurality of the plurality of fibers in the projection layer, the plurality of hollow projections extending from the outer surface of the projection layer in a direction away from the support layer; and
e. at least one ear comprising a male component of a mechanical fastening system, the at least one ear configured to releasably engage with the female component; and
f. a peel strength between the female component and the male component greater than about 150 gf.

13. The absorbent article of claim 12 wherein a second plurality of fibers of the plurality of fibers in the projection layer are entangled with the support layer.

14. The absorbent article of claim 12 wherein the projections have a height greater than about 1 mm.

15. The absorbent article of claim 12 wherein the fluid-entangled laminate web further comprises a land area which has greater than about 4% open area in a chosen area of the fluid entangled laminate web.

16. The absorbent article of claim 12 wherein the fluid-entangled laminate web further comprises a peak load in the machine direction of greater than about 3000 gf per inch.

17. The absorbent article of claim 12 wherein the fluid-entangled laminate web further comprises a fiber segment orientation rotational percent relative standard deviation of less than about 20%.

18. The absorbent article of claim 12 wherein the fluid-entangled laminate web further comprises a field anisotropy rotational percent relative standard deviation of less than about 20%.

19. The absorbent article of claim 12 wherein the fluid-entangled laminate web further comprises a percentage of void space greater than about 60%.

20. The absorbent article of claim 12 wherein the fluid-entangled laminate web has a basis weight of less than about 58 gsm.

21. The absorbent article of claim 12 wherein the peel strength between the fluid-entangled laminate web and the at least one ear is from about 150 gf to about 500 gf.

22. The absorbent article of claim 12 wherein the fluid-entangled laminate web has a peak stretch in the machine direction greater than about 20%.

23. An absorbent article comprising:
a. a bodyside liner;
b. a backsheet comprising a garment facing surface;
c. an absorbent core positioned between the bodyside liner and the backsheet;
d. a female component of a mechanical fastening system positioned on the garment facing surface of the backsheet, the female component comprising a fluid-entangled laminate web, the fluid-entangled laminate web comprising:
i. a support layer comprising opposed first and second surfaces;
ii. a projection layer comprising a plurality of fibers and opposed inner and outer surfaces, the second surface of the support layer in contact with the inner surface of the projection layer;
iii. a plurality of hollow projections formed from a first plurality of the plurality of fibers in the projection layer, the plurality of hollow projections extending from the outer surface of the projection layer in a direction away from the support layer; and
iv. a fiber segment orientation rotational percent relative standard deviation of less than about 20%.

24. The absorbent article of claim 23 wherein a second plurality of fibers of the plurality of fibers in the projection layer are entangled with the support layer.

25. The absorbent article of claim 23 wherein the projections have a height greater than about 1 mm.

26. The absorbent article of claim 23 wherein the fluid-entangled laminate web further comprises a land area which has greater than about 4% open area in a chosen area of the fluid entangled laminate web.

27. The absorbent article of claim 23 wherein the fluid-entangled laminate web further comprises a peak load in the machine direction of greater than about 3000 gf per inch.

28. The absorbent article of claim 23 wherein the fluid-entangled laminate web further comprises a peal strength greater than about 150 gf.

29. The absorbent article of claim 23 wherein the fluid-entangled laminate web further comprises a field anisotropy rotational percent relative standard deviation of less than about 20%.

30. The absorbent article of claim 23 wherein the fluid-entangled laminate web further comprises a percentage of void space greater than about 60%.

31. The absorbent article of claim 23 wherein the fluid-entangled laminate web has a basis weight of less than about 58 gsm.

32. The absorbent article of claim 23 wherein the fluid-entangled laminate web has a peak stretch in the machine direction greater than about 20%.
